(19) United States
(12) Patent Application Publication
(10) Pub. No.: US 2025/0197864 A1
(43) Pub. Date: Jun. 19, 2025

(54) LNCRNA TRANSCRIPTS IN MELANOMAGENESIS

(71) Applicant: The Regents of the University of California, Oakland, CA (US)

(72) Inventors: Susana Ortiz Urda, Oakland, CA (US); Valentin Feichtenschlager, Oakland, CA (US); Igor Vujic, Oakland, CA (US); Martina San Lorenzo, Oakland, CA (US); Linan Chen, Oakland, CA (US); Jean-Philippe Coppe, Oakland, CA (US)

(73) Assignee: The Regents of the University of California, Oakland, CA (US)

(21) Appl. No.: 18/706,957

(22) PCT Filed: Nov. 11, 2022
(86) PCT No.: PCT/US2022/049676

§ 371 Date: May 2, 2024

Related U.S. Application Data
(60) Provisional application No. 63/278,950, filed on Nov. 12, 2021.

(51) Int. Cl. C12N 15/113; A61K 9/1271; A61K 9/51; A61K 31/4725; A61K 31/473; A61K 31/506; A61K 31/519; A61K 31/55; A61P 35/00; C12N 15/86; C12N 15/88

(52) U.S. Cl. CPC C12N 15/1135; A61K 9/1271; A61K 9/5123; A61K 31/4725; A61K 31/473; A61K 31/506; A61K 31/519; A61K 31/55; A61P 35/00; C12N 15/86; C12N 15/88; C12N 2310/113; C12N 2310/14; C12N 2310/31; C12N 2310/321; C12N 2310/322; C12N 2310/3231; C12N 2310/531

(57) ABSTRACT

The invention provides compositions and methods for treatment of melanoma and other cancers. In particular, the invention provides a single or double-stranded nucleic acid that inhibits a certain group of long non-coding RNAs (lncRNAs) that have been discovered to be associated with melanoma. Inhibition of these lncRNAs in melanoma cells and xenograft mouse models leads to inhibition of cell proliferation, induction of apoptosis, and reduced cancer cell growth. The invention also relates to a method of inhibiting cancer cell growth with specific kinase inhibitors that have been found to show similar inhibition effects as the nucleic acids targeting the lncRNAs. The single or double-stranded nucleic acid and the specific kinase inhibitors constitute a novel therapeutic strategy in the treatment of melanoma and other cancers.

### BACKGROUND OF THE INVENTION

The present patent application is a U.S. 371 Application of International Application No. PCT/US2022/049676 filed Nov. 11, 2022; which claims benefit of priority to U.S. Provisional Patent Application No. 63/278,950, filed Nov. 12, 2021, which is incorporate by reference for all purposes.

### BACKGROUND OF THE INVENTION

Melanoma is the deadliest form of skin cancer and its incidence is rising [Surveillance, Epidemiology, and End Results (SEER) Program (www.seer.cancer.gov) SEER*Stat Database: Populations—Total U.S. (1969-2019)<Katrina/Rita Adjustment>—Linked To County Attributes—Total U.S., 1969-2019 Counties, National Cancer Institute, DCCPS, Surveillance Research Program, released December 2020]. Most solid tumors, including melanoma, harbor oncogene mutations which activate MAPK pathways. These important HNRNPA2signaling cascades turn extracellular stimulation into intracellular reactions and regulate cell proliferation, survival, and apoptosis. Targeting essential components of the MAPK pathway such as the BRAF and MEK kinases tremendously increased melanoma therapy progress during the last two decades. [Yuan, et al., The MAPK and AMPK signalings: interplay and implication in targeted cancer therapy. J Hematol Oncol 13, 113 (2020).; Santarpia, et al., Targeting the MAPK-RAS-RAF signaling pathway in cancer therapy. Expert Opinion on Therapeutic Targets 16, 103-119 (2012).; Attwood, M. et al, Trends in kinase drug discovery: targets, indications and inhibitor design. Nat Rev Drug Discov (2021) doi:10.1038/s41573-021-00252-y]. The antitumor effect of BRAF/MEK inhibitors and other agents relies on the stimulation of apoptosis activating pathways. [Niessner, H. et al. BRAF Inhibitors Amplify the Proapoptotic Activity of MEK Inhibitors by Inducing ER Stress in NRAS-Mutant Melanoma. Clin Cancer Res 23, 6203-6214 (2017)]. Apoptosis is a caspase dependent dissolution of cell components such as proteins and DNA. Effector caspases, like caspase 3 and 7 mediate apoptosis. The mechanisms of apoptosis involve a complex machinery of interlocking processes that can be cancer specific and negatively or positively regulated on many levels. [Carneiro, B. A. & El-Deiry, W. S. Targeting apoptosis in cancer therapy. Nat Rev Clin Oncol 17, 395-417 (2020).]. An example for an apoptosis inhibiting oncogene is hnRNPA2/B1. It is overexpressed in many types of cancer. [Gupta, A. et al. The HNRNPA2B1-MST1R-Akt axis contributes to epithelial-to-mesenchymal transition in head and neck cancer. Lab Invest (2020) doi:10.1038/s41374-020-0466-8.; Barceló, C. et al. Ribonucleoprotein HNRNPA2B1 Interacts With and Regulates Oncogenic KRAS in Pancreatic Ductal Adenocarcinoma Cells. Gastroenterology 147, 882-892.e8 (2014); Liu, et al; Identification of anti-tumoral feedback loop between VHLα and hnRNPA2B1 in renal cancer. Cell Death Dis 11, 688 (2020).; Klinge, et al; HNRNPA2/B1 is upregulated in endocrine-resistant LCC9 breast cancer cells and alters the miRNA transcriptome when overexpressed in MCF-7 cells. Sci Rep 9, 9430 (2019)]. In melanoma hnRNPA2/B1 inhibits apoptosis and could serve as potent biomarker. [Li, et al; Increased expression of YTHDF1 and HNRNPA2B1 as potent biomarkers for melanoma: a systematic analysis. Cancer Cell Int 20, 239 (2020).; Chu, et al., Requirement of splicing factor hnRNP A2B1 for tumorigenesis of melanoma stem cells. Stem Cell Res Ther 12, 90 (2021)] hnRNPA2/B1 affects apoptosis through modulating the AKT pathway and regulating caspase activity. [Barceló, C. et al. Ribonucleoprotein HNRNPA2B1 Interacts With and Regulates Oncogenic KRAS in Pancreatic Ductal Adenocarcinoma Cells. Gastroenterology 147, 882-892.e8 (2014).; Yin, et al., Effect of hnRNPA2 B1 on the proliferation and apoptosis of glioma U251 cells via the regulation of AKT and STAT3 pathways. Bioscience Reports 40, BSR20190318 (2020).; Yin, et al., Effect of hnRNPA2 B1 on the proliferation and apoptosis of glioma U251 cells via the regulation of AKT and STAT3 pathways. Bioscience Reports 40, BSR20190318 (2020).; Chen, Z.-Y. et al. Fyn requires HnRNPA2B1 and Sam68 to synergistically regulate apoptosis in pancreatic cancer. Carcinogenesis 32, 1419-1426 (2011); Jiang, F. et al. HNRNPA2B1 promotes multiple myeloma progression by increasing AKT3 expression via m6A-dependent stabilization of ILF3 mRNA. J Hematol Oncol 14, 54 (2021). Deng, J. et al. Effects of hnRNP A2/B1 Knockdown on Inhibition of Glioblastoma Cell Invasion, Growth and Survival. Mol Neurobiol 53, 1132-1144 (2016).; Yang, Y. et al. Loss of hnRNPA2B1 inhibits malignant capability and promotes apoptosis via down-regulating Lin28B expression in ovarian cancer. Cancer Letters 475, 43-52 (2020).; Peng, W. et al. hnRNPA2B1 regulates the alternative splicing of BIRC5 to promote gastric cancer progression. Cancer Cell Int 21, 281 (2021).; Chen, Z. et al. Integrative Analysis of NSCLC Identifies LINC01234 as an Oncogenic lncRNA that Interacts with HNRNPA2B1 and Regulates miR-106b Biogenesis. Molecular Therapy 28, 1479-1493 (2020).] One of the main goals of clinical oncology is the development of therapeutic agents that eradicate cancer cells by promoting apoptosis. [Carneiro, et al., Targeting apoptosis in cancer therapy. Nat Rev Clin Oncol 17, 395-417 (2020)] However, patients with initial or acquired treatment resistance point toward the limitations of existing cancer therapy. To overcome these limitations, an increased armament of anticancer therapeutics is urgently needed. [Luke, et al., Targeted agents and immunotherapies: optimizing outcomes in melanoma. Nat Rev Clin Oncol 14, 463-482 (2017)].

The majority of the human transcriptome does not get translated to proteins. A large fraction of these untranslated transcripts are long non-coding RNAs (lncRNAs), defined as non-coding complexes longer than 200 nucleotides. [Cabili, M. N. et al. Integrative annotation of human large intergenic noncoding RNAs reveals global properties and specific subclasses. Genes & Development 25, 1915-1927 (2011)]

lncRNAs can play role in oncogenesis through several mechanisms. They can regulate cancer specific gene expression as splicing factors or through epigenetic histone modification. [Amodio, N. et al. MALAT1: a druggable long non-coding RNA for targeted anti-cancer approaches. J Hematol Oncol 11, 63 (2018)] They can also promote malignant processes through activating or stabilizing protein binding partners. [Wang, S. et al. JAK2-binding long noncoding RNA promotes breast cancer brain metastasis. Journal of Clinical Investigation 127, 4498-4515 (2017).; Lin, A. et al. The LINK-A lncRNA interacts with PtdIns(3,4,5)P3 to hyperactivate AKT and confer resistance to AKT inhibitors. Nat Cell Biol 19, 238-251 (2017).] Recent research has identified and characterized novel cancer specific lncRNA transcripts. [Huarte, M. The emerging role of lncRNAs in cancer. Nat Med 21, 1253-1261 (2015).; Ding, L. et al. Role of noncoding RNA in drug resistance of prostate cancer. Cell Death Dis 12, 590 (2021).]

In recent years, an increasing number of RNA-targeting therapeutics such as Antisense Oligonucleotides (ASOs) have been brought to clinical trials and obtained FDA approval. [Bedikian, et al., Dacarbazine with or without oblimersen (a Bcl-2 antisense oligonucleotide) in chemotherapy-naive patients with advanced melanoma and low-normal serum lactate dehydrogenase: ‘The AGENDA trial’. Melanoma Research 24, 237-243 (2014).; Beer, T. M. et al. Custirsen (OGX-011) combined with cabazitaxel and prednisone versus cabazitaxel and prednisone alone in patients with metastatic castration-resistant prostate cancer previously treated with docetaxel (AFFINITY): a randomised, open-label, international, phase 3 trial. The Lancet Oncology 18, 1532-1542 (2017).] In particular, lncRNA-targeted gene silencing shows promising emerging results. [Winkle, et al., Noncoding RNA therapeutics—challenges and potential solutions. Nat Rev Drug Discov 20, 629-651 (2021).]

### BRIEF SUMMARY OF THE INVENTION

Aspects of the invention as described herein. In some aspects, the disclosure provides a single or double-stranded nucleic acid of 12-50 nucleotides in length comprising at least 12 nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, wherein introduction of the single or double-stranded nucleic acid into a cell expressing long non-coding RNA (lncRNA) BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 inhibits expression of the lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

In some embodiments, the single or double-stranded nucleic acid comprises at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

In some embodiments, the single or double-stranded nucleic acid is a single-stranded nucleic acid that is an antisense polynucleotide or a ribozyme that targets lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. In some embodiments, the single-stranded nucleic acid comprises the sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO:41 or SEQ ID NO:47.

In some embodiments, the single or double-stranded nucleic acid is a double-stranded nucleic acid that is a small interfering RNA (siRNA) or a short hairpin RNA (shRNA) that targets lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. In some embodiments, the double-stranded nucleic acid comprises a sense strand and an antisense strand, wherein the sense strand and the antisense comprise the sequence of SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 42 and SEQ ID NO: 50; SEQ ID NO: 43 and SEQ ID NO: 51; SEQ ID NO: 44 and SEQ ID NO: 52; SEQ ID NO: 45 and SEQ ID NO: 53; or SEQ ID NO: 46 and SEQ ID NO: 54.

In some embodiments, the single or double-stranded nucleic acid is a single-stranded nucleic acid that is a guide RNA (gRNA) that targets a polynucleotide encoding lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

In some embodiments, comprises at least one modified nucleotide. In some embodiments, the modified nucleotide comprises a modification selected from the group consisting of a sugar modification, a nucleic acid base modification, and a phosphate backbone modification. In some embodiments, the 2′-sugar modification is selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, and locked nucleic acid (LNA) modification. In some embodiments, the phosphate backbone modification is a 5′ phosphorylation.

In some embodiments, the double-stranded nucleic acid and comprises one or two 1-6 nucleotide (e.g., 3′) overhang.

In some aspects, the disclosure provides a vector comprising the single or double-stranded nucleic acid as described above r elsewhere herein. In some embodiments, the vector is a viral vector. In some embodiments, the viral vector is a retroviral, a lentiviral, or an adeno-associated viral (AAV) vector.

In some aspects, the disclosure provides a pharmaceutical composition comprising the single or double-stranded nucleic acid as described above or elsewhere herein or the vector as described above or elsewhere herein and a pharmaceutically acceptable carrier. In some embodiments, the pharmaceutical composition further comprises a specific inhibitor of one or more kinases selected from the group consisting of MEK, PLK1, TAF, AURKA, BER, PTK2, PKD, PKC, IKBK, MAP3K, PIM, SRC, PAK, AKT, ERK, and RAF. In some embodiments, the specific inhibitor is selected from the group consisting of trametinib, volasertib, tozasertib, alisertib, Bay-299, and CeMMEC1.

In some embodiments, the pharmaceutically acceptable carrier comprises a copolymer, a lipid, or a nanoparticle. In some embodiments, the nanoparticle is a liposomal nanoparticle.

In some aspects, the disclosure provides methods of inhibiting cancer cell. In some embodiments, the cancer cell is dependent on MAPK pathway hyperactivation. In some embodiments, the method comprises contacting the single or double-stranded nucleic acid as described above or elsewhere herein, the vector as described above or elsewhere herein, or the pharmaceutical composition as described above or elsewhere herein with the cancer cell such that expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 is inhibited.

In some embodiments, the cancer cell is a neuroblastoma ras sarcoma viral oncogene homolog (NRAS)-mutated cancer cell. In some embodiments, the cancer cell is a BRAF-mutated cancer cell.

In some embodiments, the cancer cell is in a human and the method comprises administering a therapeutically-effective amount of the single or double-stranded nucleic acid to the human.

In some embodiments, the method further comprises contacting the cancer cell with a specific inhibitor of one or more kinases selected from the group consisting of MEK, PLK1, TAF, AURKA, HER, PTK2, PKD, PKC, IKBK, MAP3K, PIM, SRC, PAK, AKT, ERK, and RAF. In some embodiments, the specific inhibitor is selected from the group consisting of trametinib, volasertib, tozasertib, alisertib, Bay-299, CeMMEC1.

In some embodiments, the method comprising contacting the cancer cell with a specific inhibitor of one or more kinases selected from the group consisting of MEK, PLK1, TAF, AURKA, HER, PTK2, PKD, PKC, IKBK, MAP3K, PIM, SRC, PAK, AKT, ERK, and RAF in an amount to inhibit the cancer cell growth. In some embodiments, the cancer cell is a neuroblastoma ras sarcoma viral oncogene homolog (NRAS)-mutated cancer cell. In some embodiments, the cancer cell is a BRAF-mutated cancer cell. In some embodiments, the specific inhibitor is selected from the group consisting of trametinib, volasertib, tozasertib, alisertib, Bay-299, and CeMMEC1.

In some embodiments, the cancer cell is in a human. In some embodiments, the cancer cell is a melanoma cell. In some embodiments, the cancer cell is a metastatic melanoma cancer cell. In some embodiments, the cancer cell is a MEK-therapy resistant cancer cell.

### BRIEF DESCRIPTION OF THE DRAWINGS

FIG. 1A-E. The lncRNA TRASH (AC004540.4) is responsive to MAPK-activation and essential for melanoma cell survival A) Schematic draft of Pipeline steps to identify MAPK responsive lncRNAs that are essential for melanoma cell survival. NRAS mutant melanocytic and melanoma cell lines were compared to wild type melanocytic cell lines and differential expressed (DE) genes were filtered for lncRNAs, high occurrence (<90%) in TCGA patient samples and essentialness in melanoma cell lines B) Venn diagram showing the transcriptome intersect of DE genes of the three comparisons PHM^{Q61}/PHM^{E}, D04/PHM^{E} and MM415/PHM^{E}. Expression change >2-fold was considered DE and 237 DE genes were filtered out. C) Scatter chart showing the percentage of expression in a TCGA patient dataset of NRAS mutant melanoma for the 119 lncRNAs derived from the list of 237 DE genes. LncRNA genes were ranked from 1 (lowest) to 119 (highest) average FPKM expression values. FPKM values >0.2 were considered as expressed. Only lncRNA genes that were expressed in >90% of patients were kept for further analysis. The red dot, highlighted with a red arrow represents TRASH. D) esiRNA respectively E) siRNA mediated silencing of TRASH affects cell viability of melanoma cell lines, but not of melanocytic cell lines. Cell viability was compared to incubation with non-targeting pooled siRNA, cells were incubated in 50 nM oligonucleotide concentration for 72 hours (n=3). ATP quantitation was used as marker for metabolically active cells. Error bars represent standard deviation, Significance shown as p-values calculated by students t-test. *=p<0.05, **=p<0.01, ***=p<0.001.

FIG. 2A-F. Biological Characterization of TRASH. A) Subcellular enrichment of lncRNAs TRASH, in D04 cells (n=3). Data was normalized to NEAT1 expression. MALAT1 (nuclear enriched) and H19 (cytoplasmic enriched) served as control. B) Relative Enrichment comparison of 4 different regions of the TRASH using primer pairs that target Exon 1 (1), Intron 1 (2), Intron1/Exon2 transition region (3) and Exon 2 (4) of the Isoform ENST00000451264.1 in D04 cells. Fold enrichment was calculated using the 2^{-ΔΔCt} method, normalized to primer pair 4 (n=3). C) Gene expression of TRASH and hnRNPA2/B1 is significantly upregulated in TCGA melanoma samples (n=469) when compared to GTEx patient samples of non-cancerous skin biopsies (n=394). Significance shown as p-values calculated by students t-test. *=p<0.05, **=p<0.01, ***=p<0.001. D) GapmeR Antisense Oligonucleotide (ASO) mediated TRASH inhibition (TRASHi) leads to significant lower TRASH expression, without significant impact on hnRNPA2/B1 expression. Gene expression fold change was measured by qRT-PCR from RNA extract of D04 cells after 24 hours of 50 nM ASO incubation (n=3) and is presented relation to Non-targeting GapmeR ASO incubation. Fold-change cut off for significant expression inhibition was considered as 0.5 (blue bar). E) Immunoblotting showing downregulation of hnRNPA2/B1 upon 1- and 2-day long ASO mediated TRASHi (100 nM) in D04 cells. Beta Actin served as loading control. Cell lysate of D04 cells incubated in non-targeting ASOs served as control. F) Left: qRT-qPCR after RIP shows >65-fold enrichment of TRASH in hnRNPA2/B1 pulldown when compared to Rabbit IgG negative control pulldown (n=3). Right: Immunoblotting showing enrichment of hnRNPA2/B1 in hnRNPA2/B1 pulldown samples compared to Rabbit IgG negative control pulldown samples. Error bars represent standard deviation.

FIG. 3A-D. Anti-apoptotic TRASH is essential for melanoma cell survival. A) Cell viability decrease upon TRASHi in the D04, MM415, WM1366, VMM39, Sk-Mel-2, WM3629, Sk-Mel-28, WM3211 standard melanoma cell lines and the Hs852.T and AV5 primary derived melanoma cell lines. Cell viability is relative to incubation with non-targeting ASOs. Incubation time was 5 days (n=3). B) Left: Colony count in the D04, MM415 and Sk-Mel-28 melanoma cell lines upon TRASHi compared to incubation with non-targeting ASOs (n=3). Right: Formed colonies in 10 cm dishes after TRASHi and incubation with non-targeting control ASOs in the D04 melanoma cell line. Incubation time was 7 days. C) Cell viability decrease upon GapmeR ASO mediated hnRNPA2/B1 (SEQ ID NO: 48) knockdown in the D04 cell line. Cell viability is relative to incubation with non-targeting ASOs. Incubation time was 5 days (n=3). D) Activity levels of the apoptosis markers Caspase 3+7 upon TRASHi and GapmeR ASO mediated hnRNPA2/B1 knockdown in the D04 cell line. Incubation time was 1 day (n=3). ASO concentration for A-D was 50 nM and in A+C ATP quantitation was used as marker for metabolically active cells. Significance is shown as p-values calculated by students t-test. *=p<0.05, **=p<0.01, ***=p<0.001. Error bars represent standard deviation.

FIG. 4A-E. TRASHi presents features of clinical value. A) Cell viability is significantly decreased upon TRASHi in the trametinib resistant melanoma cell lines D04RM, MM415RM, WM3629RM and Sk-Mel-2RM. Cell viability is relative to incubation with non-targeting ASOs. Incubation time was 5 days (n=3). Incubation concentration was 50 nM. ATP quantitation was used as marker for metabolically active cells. B) Multi drug applications of TRASHi (25 and 50 nM) and trametinib (100-0.2 nM) present combination Index (CI) values that show synergistic effects on cell viability decrease (n=2). Incubation time was 3 days. C) Tumor growth of mice that harbor xenograft (D04, AV5) and PDX (TM01341) melanoma tumors and received either systemic TRASHi or non-targeting control ASO treatment. Weight change during treatment is presented below the tumor growth curves. D) Systemic in vivo TRASHi leads to significant lower TRASH expression. Gene expression fold change was measured by qRT-PCR from RNA extracts of PDX (TM01341) tumors after 21 days of treatment (n=2) and is presented in relation to RNA levels of tumors that received non-targeting GapmeR ASO treatment. Fold-change cut off for significant expression inhibition was considered as 0.5 (blue bar). E) Left: Immunohistochemical staining for the expression of the apoptosis marker cleaved caspase 3 in D04 tumors after 21 days of systemic in vivo TRASHi (top) and non-targeting control ASO treatment (bottom). Right: Hematoxylin-eosin staining of liver tissue after 21 days of systemic in vivo TRASHi (top) and non-targeting control ASO treatment (bottom). Significance is shown as p-values calculated by students t-test. *=p<0.05, **=p<0.01, ***=p<0.001. Error bars represent standard deviation.

FIG. 5: A) Images of DAPI-, hnRNPA2/B1-, and TRASH-derived fluorescence in untreated D04 melanoma cells. Fluorescence labelling serves as visual confirmation for strong nuclear enrichment of hnRNPA2/B1 and TRASH in melanoma cells. B) Trametinib treatment causes dose dependent upregulation of hnRNPA2/B1 and TRASH expression in the D04 and MM415 cell line. C) TRASH-ASO treatment has a global effect on gene expression. Scatter plot diagram showing differential gene expression after TRASH-ASO treatment compared to Control-ASO treatment. (cut-off for significance was adjusted p-value <0.05). Data was obtained from RNA-Seq of D04 melanoma cells, treatment period was three days. D) In contrast to MEKi, TRASH-ASO treatment does not lead to drug resistance. D04 Cells that survived initial TRASH-ASO (50 nM) or MEKi (15 nM) treatment subsequently recovered in drug free media. Repetition of the preceding drug treatment in the surviving cell-subpopulation (same conditions) led to significantly increased (p=0.004) cell-growth reduction for TRASH-ASO treatment and significantly decreased (p<0.001) cell-growth reduction for MEKi treatment. Cell-growth is relative to incubation with Control-ASOs (TRASH) or drug free media (MEKi). Drug-incubation time was five days (n=3). ATP quantitation was used as marker for metabolically active cells. E) Annexin V and Propidium Iodide staining od D04 cells after 24 hours of ASO mediated TRASH inhibition confirms induction of apoptosis followed by TRASH-ASO treatment.). Significant differences of expression correlations are shown as p-values calculated by students t-test. *=p<0.05, **=p<0.01, ***=p<0.001. Error bars represent standard deviation.

FIG. 6. Mapping the phospho-catalytic signatures of TRASH-dependent cells identifies inhibition of anti-apoptotic kinases upon TRASH-ASO treatment. A) Peptide-associated phosphorylation profiles of melanoma cell-lines treated with Control-ASOs or TRASH-ASOs for one day (50 nM). Unsupervised clustering was applied (uncentered correlation and average linkage for both peptides/horizontal and samples/vertical). The profile of each sample is the average of two independent assay repeats. B) Kinase activity signatures of melanoma cell-lines treated with Control-ASOs, or TRASH-ASOs for one day (50 nM). Kinases signatures are derived from results shown in panel A). Kinases for which ≥3 biological peptides are available, are shown. Unsupervised clustering was applied as in panel a). C) Kinase activity profiles of a subset of kinases known to promote cell-survival by preventing apoptosis. Kinase activities are normalized to Control-ASO treatment per cell-line. The effect of TRASH-ASOs on these kinases is compared side-by-side to MALAT1-ASO treatment. D) MALAT1-ASO treatment inhibits cell-growth and induces apoptosis. Left: Cell-growth is significantly (p<0.001) decreased upon MALAT1-ASO treatment (50 nM) in the D04 cell-line. Cell-growth is relative to Control-ASO treatment (50 nM). Incubation time was five days (n=3). ATP quantitation was used as marker for metabolically active cells. Right: Activity levels of the apoptosis markers Caspase-3 &-7 are significantly (p=0.003) upregulated upon MALAT1-ASO treatment (50 nM) in the D04 cell-line. Caspase activity was normalized to treatment with Control-ASOs (50 nM). Incubation time was one day (n=4). and significance is shown as p-values calculated by Students t-test. *=p<0.05, **=p<0.01, ***=p<0.001. Error bars represent standard deviation. E) The specificity of the effects of TRASH-ASO treatment on the kinase activity signatures of melanoma cells is assessed in comparison to MALAT1-ASO treatment using Pearson correlation. F) Schematic summarizing the molecular impact of TRASH-ASO treatment. Expression of the lncRNA TRASH is an essential dependency that promotes the survival of melanoma cells, and that can be effectively targeted with ASOs.

FIG. 7A-D. Generating NRAS mutant melanocytic cell lines. A) Sanger Sequencing of Pooled primary human melanocytic cell lines (PHM) were equipped with an NRAS^{Q61} mutation (PHM^{Q61}), respectively an empty vector (PHM^{E}) using the Gateways entry vector pENTR/D-topo, identifies a missense mutation in codon 61 (182A>G) in NRAS in PHM^{Q61} but not in PHM^{E} B) Left: Standard microscopic imaging of PHM^{Q61} and PHM^{E} cells carrying transduction efficacy reporter vectors that co-express green fluorescent protein, right: Fluorescence microscopic imaging of same cells. Microscopic images are inn 20× magnification. C) Immunoblotting showing upregulation of NRAS an the NRAS downstream signalling effectors AKT, p-AKT, ERK, p-ERK and NRAS in PHM^{E} compared to PHM^{Q61}. GAPDH served as loading control. D) PHM^{E} and PHM^{Q61} show no significant differences in cell proliferation. ATP quantitation was used as marker for metabolically active cells and measured 5 days after seeding equal number of cells (n=3). Significant differences of expression correlations are shown as p-values calculated by students t-test. *=p<0.05, **=p<0.01, ***=p<0.001. Error bars represent standard deviation.

FIG. 8. TRASH and hnRNPA2/B1 RNA expression in melanoma and healthy skin. Expression correlation of TRASH and hnRNPA2/B1 opposed to average expression correlation of TRASH (a+c), respectively hnRNPA2/B1 (b+d) compared to 10 sets of 200 random genes in melanoma patient biopsies of the TCGA SKCM dataset (n=469, a-b) and non-cancerous skin samples from the GTEx dataset (n=394, c-d). The red line represents Spearman rank-order correlation coefficient for expression correlation in TCGA-SKCM (p=0.41, a-b) and in GTEx skin samples (ρ=0.24, c-d). Significant differences of expression correlations are shown as p-values calculated by students t-test. *=p<0.05, **=p<0.01, ***=p<0.001. Error bars represent standard deviation.

### DEFINITIONS

As used herein, the term “nucleic acid” and “polynucleotide” are used interchangeably and refer to a polymer of nucleotides, including deoxyribonucleic acids (DNA), ribonucleic acids (RNA), or any combination and polymers thereof in either single- or double-stranded form. The term encompasses nucleic acids containing modified nucleotides.

A “nucleotide”, as used herein, consists of a nucleobase, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogenous base is a derivative of purine or pyrimidine. The purines are adenine (A) and guanine (G), and the pyrimidines are cytosine (C) and thymine (T) (or in the context of RNA, uracil (U)). Nucleotides are usually mono, di- or triphosphates. A “nucleoside” is structurally similar to a nucleotide, but does not include the phosphate moieties.

The term “modified nucleotide”, as used herein refers to a nucleotide whose core structure is the same as, or closely resembles that of a nucleotide, but which has a modification, such as a sugar modification, a nucleic acid base modification and/or a phosphate backbone modification, including any known analog or derivative. A modified nucleotide may be a naturally occurring nucleotide or a non-natural nucleotide. The term “modification”, as used herein, refers to any chemical or physical modification, including substitutions and additions of chemical moieties.

As used herein, the term “complementary” or “complementarity” refer to specific base pairing between nucleotides or nucleic acids. In some embodiments, for example, and not to be limiting, base pairing between an antisense oligonucleotide and a target nucleic acid sequence in a long non-coding RNA (lncRNA) is described. Complementary nucleotides are, generally, adenine (A) and thymine (T) (or A and uracil (U)), and guanine (G) and cytosine (C). It will be understood that term “complementary” or “complementarity” also encompasses base paring between modified nucleotides, or between non-modified and modified nucleotides. In the absence of a “%” term value, complementary means fully complementary or 100% complementary. The term “% complementary” as used herein, refers to the number of nucleotides in percent of a nucleotide region or sequence in a nucleic acid (e.g. an antisense polynucleotide) which, at a given position, are complementary to (i.e. form Watson Crick base pairs with) a nucleotide sequence, at a given position of a separate nucleic acid (e.g. a lncRNA).

The term “long non-coding RNA” or “lncRNA”, as used herein refers to a non-protein coding RNA transcript that is longer than about 200 nucleotides and therefore can be distinguished from small regulatory RNAs such as microRNAs (miRNAs), small interfering RNAs (siRNAs), Piwi-interacting RNAs (piRNAs), small nucleolar RNAs (snoRNAs), and other short RNAs. In some embodiments, the lncRNA is 200 nucleotides in length. In some embodiments, the lncRNA is no longer than 200 nucleotides in length.

The term “BX470102.3”, as used herein, refers to the gene with accession number ENSG00000238279.1 in the Ensembl database. The gene is transcribed as a single isoform (or splice variant) referred herein as “BX470102.3-008” (ENST00000420695.1, SEQ ID NO: 1) with a length of 531 bp.

The term “AC004540.4”, as used herein, refers to the gene with accession number ENSG00000225792 in the Ensembl database. The gene has two isoforms referred herein as “AC004540.4-001” (ENST00000451368; SEQ ID NO: 2) with a length of 611 bp, and “AC004540.4-002” (ENST00000451264; SEQ ID NO: 3) with a length of 508 bp.

The term “RP11-7011.3”, as used herein, refers to the gene with accession number ENSG00000237950.1 in the Ensembl database. The gene has three isoforms referred herein as “RP11-7011.3-001” (ENST00000446167.1; SEQ ID NO: 4) with a length of 486 bp, “RP11-7011.3-003” (ENST00000445226.1; SEQ ID NO: 5) with a length of 294 bp, and “RP11-7011.3-002” (ENST00000412378.1; SEQ ID NO: 6) with a length of 494 bp.

The term “RN7SL1”, as used herein, refers to the gene with accession number ENSG00000258486.1 in the Ensembl database. The gene has two isoforms referred herein as “RN7SL1-202” (ENST00000635274.1; SEQ ID NO: 7) with a length of 300 bp, and “RN7SL1-201” (ENST00000618786.1; SEQ ID NO: 8) with a length of 299 bp.

The term “ARF-AS1”, as used herein, refers to the gene with accession number ENSG00000272146 in the Ensembl database. The gene has three isoforms referred herein as “ARF-AS1-201” (ENST00000606192.5; SEQ ID NO: 9) with a length of 327 bp, “ARF-AS1-202” (ENST00000607297.1; SEQ ID NO: 10) with a length of 437 bp, and “ARF-AS1-203” (ENST ENST00000607782.1; SEQ ID NO: 11) with a length of 552 bp.

The term “AL157871.4”, as used herein, refers to the gene with accession number ENSG00000258666 in the Ensembl database. The gene is transcribed as a single isoform referred herein as “AL157871.4-201” (ENST00000557226.1; SEQ ID NO: 12) with a length of 385 bp.

As used herein, the term “inhibition”, or any grammatical variation thereof (e.g., inhibit, inhibiting, etc.) as referred to herein, relates to the retardation, restraining or reduction of the lncRNA levels, expression and/or activity by the nucleic acids of the invention and the specific kinase inhibitors by at least 5%, at least 10%, at least 20%, at least 30%, at least, at least 40%, at least 50%, at least 60%, at least 70%, at least 80%, at least 90%, or 100%, or any percentage in between.

As used herein, an “antisense polynucleotide”, “antisense oligonucleotide” or “ASO” is a single-stranded nucleic acid sequence (DNA, RNA, or a nucleotide analog) capable of hybridizing to a target RNA sequence (e.g., a lncRNA). Upon binding to their target RNA, ASOs can inhibit gene expression and/or initiate the degradation of the target RNA through various mechanisms, for example by inducing cleavage of the target RNA through endoribonuclease (RNase) recruitment.

As used herein “ribozymes” are catalytic RNA oligonucleotides that can bind to a target RNA and cleave the target RNA through various cleavage mechanisms. Generally, ribozymes comprise a catalytic region and one or more binding regions. The binding regions hybridize to a complementary sequence of the target RNA, and the catalytic region cleaves the target RNA.

The term “hybridizes” or any grammatical variation thereof (e.g., hybridizing, hybridization, etc.) and “bind” or any grammatical variation thereof (e.g., binding, etc.) are used interchangeably and refer to the annealing of two nucleic acids strands. In particular, two nucleic acid strands form hydrogen bonds between base pairs of the two strands, thereby forming a duplex. In certain embodiments, an antisense oligonucleotide, an siRNA, or a shRNA may hybridize with a target nucleic acid sequence contained in a lncRNA.

As used herein “target sequence” or “target nucleic acid sequence” refers to a particular nucleotide sequence of the target nucleic acid to which a comnplementary nucleic acid binds to. In certain embodiments, the target sequence may be contained in the lncRNAs or a polynucleotide encoding one of the lncRNAs as described hereini

The term “target” or any grammatical variation thereof (e.g., targeting etc.) refers to the capability of a nucleic acid to bind to or hybridize with a target sequence on a complementary nucleic acid strand and inhibit its expression, reduce its levels and/or activity.

As used herein, the term “small interfering RNA (siRNA)” refers to a double-stranded RNA (or RNA analog) that is capable of directing or mediating RNA interference. In some embodiments, the siRNA is 10-50 nucleotides (or nucleotide analogs), e.g., 12-30 nucleotides in length, e.g., 15-25 nucleotides in length, e.g., 19-23 nucleotides in length, e.g., 21-23 nucleotides in length. Therefore, exemplary siRNA molecules are 12, 13, 14, 15, 16, 17, 18, 19, 20, 21, 22, 23, 24, 25, 26, 27 28 or 29 nucleotides in length. In certain embodiments, the siRNA is a 21-mer comprising 21 nucleotides.

The term “short hairpin RNA”, “small hairpin RNA”, and “shRNA” are used interchangeably and refer to a double-stranded interfering RNA (e.g., siRNA) where the two strands are connected to form a hairpin or loop region.

The term “antisense strand” refers to the strand of the siRNA or shRNA that contains some degree of complementarity to the target sequence. As used herein, the term “sense strand” refers to the strand of the siRNA or shRNA that contains complementarity to the antisense strand.

As used herein, the term “overhang” refers to a single-stranded portion of a double-stranded nucleic acid that extends beyond the terminus of the complementary strand of the double-stranded nucleic acid.

The term “guide RNA” or “gRNA”, as used herein refers to a nucleic acid that binds to a Cas protein and aids in targeting the Cas protein to a specific target sequence within DNA. A gRNA may comprise a crisp RNA (crRNA) and a transactivating crisp RNA (tracrRNA).

The term “vector”, as used herein, refers to a nucleic acid molecule capable of transporting another nucleic acid to which it has been linked. One type of vector is a “viral vector” comprising virus derived sequences used to deliver a nucleic acid (e.g. an antisense oligonucleotide, an siRNA or shRNA, a ribozyme, or a gRNA) to a cell.

The term “specific inhibitor”, as used herein refers to a compound that interacts with a certain kinase or a certain group of kinases and inhibits the enzymatic activity of that specific kinase or that specific group of kinases, but does not significantly interact with and inhibit the enzymatic activity of other kinases.

“Pharmaceutically acceptable carrier” and “pharmaceutically acceptable excipient” are used interchangeably and refer to a substance or compound that aids or facilitates preparation, storage, administration, delivery, effectiveness, absorption by a subject, or any other feature of the composition for its intended use or purpose. Such pharmaceutically acceptable carrier is not biologically or otherwise undesirable and can be included in the compositions of the present invention without causing a significant adverse toxicological effect on the subject or interacting in a deleterious manner with the other components of the pharmaceutical composition.

As used herein, the term “administering”, “administration”, or “administer” means delivering the pharmaceutical composition as described herein to a target cell or a subject (e.g., a human). The pharmaceutical compositions described herein are designed for delivery to subjects in need thereof by any suitable route or a combination of different routes. In particular embodiments, pharmaceutical compositions are administered by intratumoral injection.

The term “neuroblastoma ras sarcoma viral oncogene homolog (NRAS)-mutated cancer cell” or “neuroblastoma ras sarcoma viral oncogene homolog (NRAS)-mutated cancer”, as used herein, refers to a cancer cell or cancer that comprises a NRAS mutation. A “NRAS mutation”, as used herein, refers to a mutation that occurs on a gene located in humans on chromosome 1 and which encodes the small GTPase Ras family protein neuroblastoma ras sarcoma viral oncogene homolog (NRAS).

The term “v-Raf murine sarcoma viral oncogene homolog B1 (BRAF)-mutated cancer cell” or “v-Raf murine sarcoma viral oncogene homolog B1 (BRAF)-mutated cancer”, as used herein, refers to a cancer cell or cancer that comprises a BRAF mutation. A “BRAF mutation”, as used herein, refers to a mutation that occurs on a gene located in humans on chromosome 7 and which encodes the B-Raf protein.

As used herein, the term “cancer” refers to all types of cancer, neoplasm or malignant tumors found in mammals, including leukemia, carcinomas and sarcomas.

“Tumor,” as used herein, refers to all neoplastic cell growth and proliferation and cancerous cells and tissues.

As used herein, the term “melanoma” refers to a form of skin cancer that may affect the skin only or may spread (metastasize) through the blood or lymph systems to organs and bones. Melanoma can develop in an existing mole or other mark on the skin or on unmarked skin. As used herein, the term “metastatic melanoma” refers to melanoma that has spread to other tissues or organs.

“MEK-therapy resistant cancer cell”, as used herein, refers to a cancer cell that does not respond to a MEK therapy (such as a therapy including a MEK inhibitor). The cancer cell may be intrinsically resistant to a MEK therapy or may have acquired resistance to a MEK therapy.

“MAPK-therapy resistant cancer cell”, as used herein, refers to a cancer cell that does not respond to a MAPK therapy (such as a therapy including a MAPK inhibitor). The cancer cell may be intrinsically resistant to a MAPK therapy or may have acquired resistance to a MAPK therapy.

“BRAY-therapy resistant cancer cell”, as used herein, refers to a cancer cell that does not respond to a BRAF therapy (such as a therapy including a BRAF inhibitor). The cancer cell may be intrinsically resistant to a BRAF therapy or may have acquired resistance to a BRAF therapy.

### DETAILED DESCRIPTION OF THE INVENTION

### 1. Introduction

Recently, genomic studies have identified a class of non-protein-coding RNAs lacking protein-coding capacity, defined as long non-coding RNAs (lncRNAs). They have been shown to be involved in a variety of transcriptional and post-transcriptional gene regulatory processes through multiple mechanisms. The inventors have developed compositions and methods for treatment of melanoma and other NRAS-mutated cancers, inter alia, by delivering nucleic acids that inhibit the expression of a certain group of lncRNAs newly associated with cancer. In particular, the inventors have discovered that inhibiting lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 reduces the oncogenic phenotype of melanoma, exemplified as reduced in vitro proliferation, increased apoptosis, as well as reduced tumor growth in a xenograft mouse model of melanoma. Further, the inventors discovered a certain group of kinases that is downregulated as a result of lncRNA inhibition. Specifically, the inventors discovered that inhibiting these specific kinases mimics the inhibition effects of the lnRNAs and leads to significant reduction in cell viability. Moreover, the inventors demonstrate improved effects when combining specific kinase inhibitors with antisense oligonucleotides (ASOs) that target the lncRNAs. Finally, lncRNA knockdown experiments in other cancer cell lines indicate targeting these lncRNAs are effective in treating other cancer types as well.

Accordingly, the disclosure provides a single or double-stranded nucleic acid that inhibits expression of the lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. As described herein, a series of novel antisense oligonucleotides (ASOs) and small interfering RNA (siRNAs) have been developed that target the specific lncRNAs.

In some approaches, the disclosure provides a method of inhibiting a cancer cell. In some embodiments, the cancer cell is a neuroblastoma ras sarcoma viral oncogene homolog (NRAS)-mutated cancer cell. In some embodiments, the cancer cell is a v-Raf murine sarcoma viral oncogene homolog B1 (BRAF)-mutated cancer cell. In some aspects, the method involves contacting the single or double-stranded nucleic acid with the cancer cell such that expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 is inhibited. In one approach, the method involves administering a therapeutically-effective amount of the single or double-stranded nucleic acid to a human. In some embodiments, the human is in need of treatment. In some aspects, the human has cancer. In one embodiment, the human has skin cancer, such as melanoma. In some embodiments, the cancer is an astrocytoma, a glioblastoma, a neuroblastoma, multiple myeloma, a small cell lung cancer, a large cell carcinoma, optionally from lung, a non-small cell lung cancer, a colon adenocarcinoma or an osteosarcoma.

In some embodiments, the method further comprises contacting the cancer cell with a specific inhibitor of MEK, PLK1, TAF, AURKA, HER, PTK2, PKD, PKC, IKBK, MAP3K, PIM, SRC, PAK, AKT, ERK, or RAF kinase.

Aspects of the disclosure further relate to a method of inhibiting a cancer cell (e.g., a NRAS-mutated cancer cell or a BRAF-mutated cancer cell), where the method involves contacting the cancer cell with a specific inhibitor of MEK, PLK1, TAF, AURKA, HER, PTK2, PKD, PKC, IKBK, MAP3K, PIM, SRC, PAK, AKT, ERK, or RAF kinase in an amount to inhibit the cancer cell growth. In one approach, the cancer cell is in a human, and the human is in need of treatment. In some aspects, the human has cancer. In one embodiment the human has skin cancer, such as melanoma. In some embodiments, the cancer is an astrocytoma, a glioblastoma, a neuroblastoma, multiple myeloma, a small cell lung cancer, a large cell carcinoma, optionally from lung, a non-small cell lung cancer, a colon adenocarcinoma or an osteosarcoma.

### 2. Inhibiting Expression of lncRNAs

In some aspects, the invention provides a single or double-stranded nucleic acid that inhibits expression of the lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. In some embodiment, the single or double-stranded nucleic comprises a sequence complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the single or double-stranded nucleic acid is 8-100, e.g., 12-50, e.g., 16-30 nucleotides in length. In some aspects, the single or double-stranded nucleic acid comprises at least 8, at least 9, at least 10, at least, 11, at least, 12, at least 13, at least 14, at least 15, or at least 16 nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some aspects, the single or double-stranded nucleic acid comprises at least 8, at least 9, at least 10, at least, 11, at least, 12, at least 13, at least 14, at least 15, or at least 16 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some aspects, the single or double-stranded nucleic acid comprises 8, 9, 10, 11, 12, 13, 14, 15, or 16 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

The complementarity between a nucleic acid and its corresponding target sequence may be 100%. In some embodiments, the complementarity between a nucleic acid and its corresponding target sequence is less than 100%, although 100% complementarity is desired to avoid off-target effects. In some embodiments, the complementarity between a nucleic acid and its corresponding target sequence is at least 95%, at least 90%, at least 85%, or at least 80%.

Introduction of the single or double-stranded nucleic acid into a cell expressing lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 inhibits expression of the lncRNA BX470102.3-008, AC004540.4-001, AC004540.4-002, RP11-7011.3-001, RP11-7011.3-003, RP11-7011.3-002, RN7SL1-xxx, RN7SL1-201, ARF-AS1-201, ARF-AS1-202, ARF-AS1-203 or AL157871.4-201. In some embodiments, the inhibition of expression is at least 5% compared to the normal expression level in a cell expressing lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. Normal expression levels can be assessed in a control without the introduction of the single or double-stranded nucleic acid, e.g., as described herein. In some embodiments, the inhibition of expression is at least 5%, at least 10%, at least 20%, at least 30%, at least, at least 40%, at least 50%, at least 60%, at least 70%, at least 80%, at least 90%, or 100%, or any percentage in between. Ideally, the inhibition of expression is between 95% and 100%.

The single or double-stranded nucleic acid can act at the DNA level or at the RNA level to inhibit the expression of the lncRNAs. Any suitable method may be used to achieve such inhibition. For example, inhibition at the RNA level may involve the use of antisense oligonucleotides (ASOs), ribozymes, or gene silencing methods in the form of RNA interference (RNAi). Inhibition at the DNA level may be performed through CRISPR/Cas systems using guide RNAs (gRNA). These and other compounds will be further detailed herein below.

### 2.1 Antisense Oligonucleotides

In some aspects, the single or double-stranded nucleic acid is a single-stranded nucleic acid that is an antisense polynucleotide that targets and binds to lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. The antisense polynucleotide or antisense oligonucleotide (ASO) specifically hybridizes with the lncRNA and reduces levels of lncRNA BX470102.3-008, AC004540.4-001, AC004540.4-002, RP11-7011.3-001, RP11-7011.3-003, RP11-7011.3-002, RN7SL1-xxx, RN7SL1-201, ARF-AS1-201, ARF-AS1-202, ARF-AS1-203 or AL157871.4-201. In some embodiment, the antisense polynucleotide comprises a sequence complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the antisense polynucleotide is 8-100, e.g., 12-50, e.g., 16-30 nucleotides in length. In some embodiments, the antisense polynucleotide is 16 nucleotides in length. In some embodiments, the antisense polynucleotide comprises at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the antisense polynucleotide comprises at least 8, at least 9, at least 10, at least 11, at least 12, at least 13, at least 14, at least 15, or at least 16 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the antisense polynucleotide comprises the sequences of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO:41 or SEQ ID NO:47.

In some embodiments, an antisense polynucleotide comprising SEQ ID NO: 48 is provided, wherein introduction of the antisense polynucleotide into a cell expressing HNRNPA2/B1 inhibits expression of HNRNPA2/B1. In some embodiments, the antisense polynucleotide is 8-100, e.g., 12-50, e.g., 16-30 nucleotides in length. In some embodiments, the antisense polynucleotide is 16 nucleotides in length.

In some embodiments, an antisense polynucleotide comprising SEQ ID NO: 49, wherein introduction of the antisense polynucleotide into a cell expressing SNX10 inhibits expression of SNX10. In some embodiments, the antisense polynucleotide is 8-100, e.g., 12-50, e.g., 16-30 nucleotides in length. In some embodiments, the antisense polynucleotide is 16 nucleotides in length.

The complementarity between an antisense polynucleotide and its corresponding target sequence may be 100%. In some embodiments, the complementarity between the antisense polynucleotide and its corresponding target sequence is less than 100%, although 100% complementarity is desired to avoid off-target effects. In some embodiments, the complementarity between the antisense polynucleotide and its corresponding target sequence is at least 95%, at least 90%, at least 85%, or at least 80%.

In some embodiments, the antisense oligonucleotide comprises one or more modified nucleotides. In some embodiments, the modified nucleotide comprises a sugar modification, a nucleic acid base modification, and/or a phosphate backbone modification. Exemplary modifications are described further below. In one particular embodiment, the antisense polynucleotide is designed as a gapmer comprising a central stretch (gap) of nucleotides capable of inducing RNase H cleavage, and two flanking regions containing one or more modified nucleosides. Gapmer structures are well characterized and may be designed using known methods in the art, see, e.g., Monia et al. (1993), “Evaluation of 2”—modified oligonucleotides containing 2—“deoxy gaps as antisense inhibitors of gene expression”, J. Biol. Chem.; 268:14514-14522; Deleavey et al. (2012), “Designing chemically modified oligonucleotides for targeted gene silencing”, Chem. Biol.; 19:937-954; and Stanley T. Crooke (2008), “Antisense Drug Technology—Principles, Strategies, and Applications”, 2nd Edition, CRC press. Accordingly, in some aspects, the antisense polynucleotide is a gapmer. In some embodiments, the antisense polynucleotide is a locked nucleic acid (LNA) gapmer, where the modified nucleotides in the flanking regions are LNA nucleotides. In some embodiments, the antisense polynucleotide is a mixmer comprising alternating stretches of LNA and unmodified nucleotides, see e.g. U.S. Pat. Nos. 5,013,830; 5,149,797; 5,220,007; 5,256,775, each of which is herein incorporated by reference. In one embodiment, the antisense polynucleotide is a headmer comprising only a flanking region at the 5′ terminus. In another embodiment, the antisense polynucleotide is a tailmer comprising only a flanking region at the 3′ terminus.

In some embodiments, the antisense polynucleotide comprises 1-8, e.g., 2-6 LNA nucleotides. In some embodiments, the antisense polynucleotide comprises 1, 2, 3, 4, 5, 6, 7, or 8 LNA nucleotides.

### 2.2 Ribozymes

In some embodiments, the single or double-stranded nucleic acid is a single-stranded nucleic acid that is a ribozyme that targets and binds to lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. Ribozymes are catalytic RNA oligonucleotides with enzyme-like cleavage properties that bind and cleave target RNAs. Ribozyme structures useful for targeting the lncRNAs as described herein include hammerhead ribozymes and hairpin ribozymes, and are characterized, for example, in Citti and Rainaldi (2005), “Synthetic hammerhead ribozymes as therapeutic tools to control disease genes”, Curr Gene Ther.; 5(1):11-24; Hean & Weinberg (2008), “The Hammerhead Ribozyme Revisited: New Biological Insights for the Development of Therapeutic Agents and for Reverse Genomics Applications”, In Morris KL (ed.). RNA and the Regulation of Gene Expression: A Hidden Layer of Complexity. Norfolk, England: Caister Academic Press; Usman and McSwiggen, “Ch. 30—Catalytic RNA (Ribozymes) as Drugs,” Annual Reports in Medicinal Chemistry 30:285-294 (1995). In general, a ribozyme comprises a target binding portion that hybridizes to a target sequence of RNA and an enzymatic portion that acts to cleave the target RNA.

Accordingly, in some embodiment, the ribozyme comprises a sequence complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the ribozyme polynucleotide is 8-100, e.g., 12-50 nucleotides in length. In some embodiments, the ribozyme comprises at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the ribozyme comprises at least 8, at least 9, at least 10, at least 11, at least 12, at least 13, at least 14, at least 15, or at least 16 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

The complementarity between a target binding portion of a ribozyme and its corresponding target sequence may be 100%. In some embodiments, the complementarity between target binding portion of a ribozyme and its corresponding target sequence is less than 100%, although 100% complementarity is desired to avoid off-target effects. In some embodiments, the complementarity between the target binding portion of a ribozyme and its corresponding target sequence is at least 95%, at least 90%, at least 85%, or at least 80%.

In some embodiments, the ribozyme comprises one or more modified nucleotides. Such modified nucleotides may comprise a sugar modification, a nucleic acid base modification, and/or a phosphate backbone modification. Exemplary modifications include those described for antisense oligonucleotides (see above) or those described in § 2.5, below.

### 2.3 RNA Interference

In some embodiments, the single or double-stranded nucleic acid is a double-stranded nucleic acid that is a small interfering RNA (siRNA) or a small hairpin RNA (shRNA) that targets lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. siRNA and shRNA are involved in the RNA interference (RNAi) pathway where they can induce degradation of a target RNA. Methods for constructing siRNAs useful for inhibiting target RNAs are known to those of skill in the art, see e.g., Fire et al. (1998), “Potent and specific genetic interference by double-stranded RNA in Caenorhabditis elegans”, Nature, 391:806-811; Elbashir et al. (2001), “Duplexes of 21-nucleotide RNAs mediate RNA interference in cultured mammalian cells”, Nature, 411:494-498; Brummelkamp (2002), “A System for Stable Expression of Short Interfering RNAs in Mammalian Cells”, Science, 296:550-553; Wittrup and Lieberman (2015), “Knocking down disease: a progress report on siRNA therapeutics”, Nature Rev Genet., 16:543-552; Vickers et al. (2003), “Efficient Reduction of Target RNAs by Small Interfering RNA and RNase H-dependent Antisense Agents”, J. Biol. Chem., 278:7108-7118. siRNAs comprise a sense strand and a complementary antisense strand annealed together by standard Watson Crick base pairing interactions. The sense strand may comprise a nucleic acid sequence that is identical to a target sequence contained within a target RNA, and the antisense strand may comprise a nucleic acid sequence that is complementary to a target sequence contained within the target RNA. In the case of the shRNA, the sense and antisense strand are covalently linked by a single-stranded loop region, and the shRNA is converted into a siRNA by a cleavage event mediated by the enzyme Dicer. The loop region may be between 2 and 12 nucleotides in length. In some cases, the loop region is from 4 to 10 nucleotides in length. Details on the structure of shRNAs can be found, for example, in Paddison et al. (2002), “Short hairpin RNAs (shRNAs) induce sequence-specific silencing in mammalian cells”, Genes Dev., 16(8):948-958; Brummelkamp (2002), Science, 296:550-553; and Yu et al. (2002), “RNA interference by expression of short-interfering RNAs and hairpin RNAs in mammalian cells”, Proc Natl Acad Sci USA, 99:6047-6052). siRNAs associate with an endonuclease-containing complex, known as RNA-induced silencing complex (RISC). RISC specifically recognizes and cleaves the target RNA that contains a nucleic acid sequence complementary to the antisense strand.

Accordingly, in some embodiments, the siRNA or shRNA that targets and binds to the lncRNA as described herein comprises a sequence complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the siRNA or shRNA is 8-100, e.g., 12-50, e.g., 16-30, e.g., 19-25 nucleotides in length. In some embodiments, the siRNA or shRNA is 21 nucleotides in length. In some embodiments, the siRNA or shRNA comprises at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the siRNA or shRNA comprises at least 8, at least 9, at least 10, at least 11, at least 12, at least 13, at least 14, at least 15, at least 16, at least, 17, at least, 18, at least 19, at least 20, at least 21, at least 22, at least 23, at least 24, or at least 25 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

In some aspects, the siRNA or shRNA comprises a sense strand and an antisense strand, where the sense strand and the antisense comprise the sequence of SEQ ID NO: 23 and SEQ ID NO: 24, respectively; SEQ ID NO: 25 and SEQ ID NO: 26, respectively; SEQ ID NO: 27 and SEQ ID NO: 28, respectively; SEQ ID NO: 29 and SEQ ID NO: 30, respectively; SEQ ID NO: 31 and SEQ ID NO: 32, respectively; SEQ ID NO: 33 and SEQ ID NO: 34, respectively; SEQ ID NO: 35 and SEQ ID NO: 36, respectively; SEQ ID NO: 37 and SEQ ID NO: 38, respectively; SEQ ID NO: 39 and SEQ ID NO: 40, respectively; SEQ ID NO: 42 and SEQ ID NO: 50, respectively; SEQ ID NO: 43 and SEQ ID NO: 51, respectively; SEQ ID NO: 44 and SEQ ID NO: 52, respectively; SEQ ID NO: 45 and SEQ ID NO: 53, respectively; or SEQ ID NO: 46 and SEQ ID NO: 54, respectively.

The complementarity between an siRNA or shRNA and its corresponding target sequence may be 100%. In some embodiments, the complementarity between the siRNA or shRNA and its corresponding target sequence is less than 100%, although 100% complementarity is desired to avoid off-target effects. In some embodiments, the complementarity between the siRNA or shRNA and its corresponding target sequence is at least 95%, at least 90%, at least 85%, or at least 80%.

In some embodiments, the siRNA or shRNA comprises one or more modified nucleotides. In some embodiments, the modified nucleotide of the siRNA or shRNA comprises a sugar modification, a nucleic acid base modification, and/or a phosphate backbone modification. Exemplary modifications are described further below. In one particular embodiment, the siRNA or shRNA includes one or more locked nucleic acids (LNA). A locked nucleic acid is a nucleotide having a modified ribose moiety in which the ribose moiety comprises an extra bridge connecting the 2′ and 4′ carbons. See e.g., Elmen et al. (2005), Nucleic Acids Research 33(1):439-447; Mook et al. (2007), Mol Canc Ther 6(3):833-843; Grunweller et al. (2003), Nucleic Acids Research 31(12):3185-3193).

In some embodiments, the siRNA or shRNA comprises an overhang on either the sense strand or the antisense strand or both (e.g., on each 3′ end of both strands). In some embodiments, siRNA or shRNA includes an overhang on both the sense and the antisense strand. The overhang may be at either the 5′ end or the 3′ end of the strand In some embodiments, both the 5′ end and the 3′ end comprise an overhang. The overhang can have any nucleotide sequence and may be 1-10 nucleotides in length. In some embodiments, the overhang is 2-6 nucleotides in length. In some embodiments, the overhang is 2-4 nucleotides in length. In some cases, the overhang comprises modified nucleotides. For example, the overhang may include locked nucleic acids (LNAs).

2.4 CRISPR/Cas systems

In some approaches, CRISPR technology is used to inhibit expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. The CRISPR technology is a gene-editing method that makes use of the CRISPR/CAS system. The “CRISPR/Cas” system refers to a widespread class of bacterial systems for defense against foreign nucleic acid. CRISPR/Cas systems include type I, II, and III sub-types. Wild-type type II CRISPR/Cas systems use the RNA-mediated nuclease, for example, Cas9, in complex with guide and activating RNA to recognize and cleave foreign nucleic acid. In nature, many CRISPR systems include transactivating crisp RNA (tracrRNA), which binds the Cas endonuclease, and crisp RNA (crRNA), which binds to the DNA target sequence. Some CRISPR systems (e.g., CRISPR Cas12a/Cpf1) require only crRNA. In research and biomedical applications it is more typical to use a chimeric single guide RNA (“sgRNA”), which is a crRNA-tracrRNA fusion that binds both the Cas endonuclease and the DNA target sequence. It will be understood that, except where apparent from context, reference to a “gRNA” includes any suitable guide RNA with appropriate binding specificity (e.g., a sgRNA, crRNA, or other RNA that binds to any of the genes encoding the lncRNAs of interest). The most commonly used sgRNA's comprise a nucleic acid sequence approximately 20 nucleotides in length which is complementary to a target sequence, and which is located at or near the 5′ end of the sgRNA. Methods for designing sgRNAs that target a specified target sequence are well known in the art. See e.g., Doench et al. (2016), Optimized sgRNA design to maximize activity and minimize off-target effects of CRISPR-Cas9”, Nat. Biotechnol. 34:184-191; Horlbeck et al. (2016), “Compact and highly active next-generation libraries for CRISPR-mediated gene repression and activation, eLife. 5, e19760 (2016); Cui et al., “Review of CRISPR/Cas9 sgRNA Design Tools. Interdiscip. Sci. 2018, 10:455-465; and Kiani et al. (2015), “Cas9 gRNA engineering for genome editing, activation and repression”, Nat Methods 2015; 12:1051-4.

Aspects of the invention relate to a single-stranded nucleic acids that is a guide RNA (gRNA) that targets a polynucleotide encoding lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. In some embodiments, the polynucleotide is BX470102.3, AC004540.4, RP11-7011.3, RN7SL1, ARF-AS1, or AL157871.4.

In some aspects, introduction of the gRNA in a cell expressing lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 inhibits expression of the lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201. In some embodiments, the gRNA is of 20 nucleotides in length. In some embodiments, the gRNA comprises at least 12, at least 15, or at least 20 nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the gRNA comprises at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some cases, the guide RNA is an sgRNA. In some embodiments, the gRNA comprises at least 8, at least 9, at least 10, at least 11, at least 12, at least 13, at least 14, at least 15, at least 16, at least 17, at least 18, at least 19, or at least 20 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some cases, the guide RNA is an sgRNA.

The complementarity between a gRNA and its corresponding target sequence may be 100%. In some embodiments, the complementarity between the gRNA and its corresponding target sequence is less than 100%, although 100% complementarity is desired to avoid off-target effects. In some embodiments, the complementarity between the gRNA and its corresponding target sequence is at least 95%, at least 90%, at least 85%, or at least 80%.

In some embodiments, the gRNA comprises one or more modified nucleotides. In some embodiments, the modified nucleotide comprises a sugar modification, a nucleic acid base modification, and/or a phosphate backbone modification. gRNAs comprising modified nucleotides are described, for example in WO2018107028. See also e.g., Filippova et al. (2019), “Guide RNA modification as a way to improve CRISPR/Cas9-based genome-editing systems”, Biochimie., 167:49-60; Ryan et al. (2018), “Improving CRISPR-Cas specificity with chemical modifications in single-guide RNAs”, Nucleic Acids Res. 46, 792-803; and Hendel et al. (2015), “Chemically modified guide RNAs enhance CRISPR-Cas genome editing in human primary cells”. Nat. Biotechnol. 2015; 33:985-989. Additional modifications that may be used are described further below.

In some aspects, the invention relates to a CRISPR/Cas system, where the system comprises a Cas protein and a guide RNA (e.g., an sgRNA) as described above. The sgRNA and Cas can be expressed from the same or different vectors of the system. Cas proteins and their amino acid sequence are well known in the art. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. An exemplary Cas9 protein is the Streptococcus pyogenes Cas9 protein. The amino acid sequence of S. pyogenes Cas9 protein may be found in the SwissProt database under accession number Q99ZW2. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. In some embodiments, the Cas (such as Cas9) lacks nuclease activity (e.g., dCas9). In some cases, the CRISPR/Cas system comprises a Cas fusion protein including a Cas DNA binding domain and a transcription repressor. In some cases, the Cas is a nuclease deficient dCas (such as dCas9). Other RNA-mediated nucleases that can also be used in a CRISPR/Cas system to inhibit the expression of the lncRNAs include, for example, Cas 12a and Cascade/Cas3 (see e.g., Pickar-Oliver and Gersbach (2019), “The next generation of CRISPR-Cas technologies and applications”, Nat. Rev. Mol. Cell Biol., 20: 490-507).

In some cases, the gRNA binds to a target sequence that is contiguous with a protospacer adjacent motif (PAM) recognized by the Cas protein. For example, Cas9 generally requires the PAM motif NGG for activity. Thus, in some systems, certain target sequences will be preferred based on the proximity of the target sequence to a PAM. However, some Cas proteins, including variants of Cas9, have flexible PAM requirements (see Karvekis et al., 2019, “PAM recognition by miniature CRISPR-Cas14 triggers programmable double-stranded DNA cleavage.” bioRxiv.; Legut et al., 2020, “High-Throughput Screens of PAM-Flexible Cas9”, Cell Reports 30:2859-2868; Gleditzsch et al., 2019, PAM identification by CRISPR-Cas effector complexes: diversified mechanisms and structures. RNA Biol. 2019 April; 16(4): 504-517) and other Cas proteins are PAM-independent (e.g., Cas14a1). Exemplary PAMs are described, e.g., in Zhao et al. (2017), CRISPR-offinder: a CRISPR guide RNA design and off-target searching tool for user-defined protospacer adjacent motif. Int J Biol Sci; 13(12):1470-1478.

### 2.5 Modifications to the Nucleic Acids

In some aspects, the single or double-stranded nucleic acid of the present disclosure may include one or more modified nucleotides to improve certain properties of the nucleic acids, such as binding affinity, stability, and/or nuclease resistance. Accordingly, in some embodiments, the single or double-stranded nucleic acid of the present disclosure comprises at least one nucleotide that is modified. In some embodiments, the antisense oligonucleotide comprises at least one modified nucleotide. In some embodiments, the ribozyme comprises at least one modified nucleotide. In some embodiments, the siRNA or shRNA comprises at least one modified nucleotide. In some embodiments, the gRNA comprises at least one modified nucleotide. In some aspects, the modified nucleotide comprises a sugar modification, a nucleic acid base modification, and/or a phosphate backbone modification. Modifications that are useful for optimizing the single or double-stranded nucleic of the present disclosure are described, e.g., in Freier & Altmann (1997), Nucl. Acid Res., 25, 4429-4443; Uhlmann (2000), Curr. Opinion in Drug Development, 3(2), 293-213; and Deleavey and Damha (2012), Chemistry and Biology, 19: 937-954, and U.S. Pat. Nos. 5,684,143, 5,858,988 and 6,291,438. Below are some exemplary modifications that may be incorporated.

Sugar modifications include alternations of the substituent groups on the ribose ring to groups other than hydrogen, or the 2′—OH group naturally found in DNA and RNA nucleosides. Substituents may, for example be introduced at the 2′, 3′, 4′ or 5′ positions. In some embodiments, the single or double-stranded nucleic acid of the present disclosure comprises at least one 2′ sugar modification. A 2′ sugar modification comprises any modification made at the 2′ position of the sugar, where the nucleotide comprises a substituent other than H or —OH at the 2′ position of the sugar. For example, the 2′ modified sugar may provide enhanced binding affinity and/or increased nuclease resistance to the oligonucleotide. In some embodiments, the 2′ sugar modification is a 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, and locked nucleic acid (LNA) modification.

Sugar modifications may also include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). In some embodiments, modifications comprise an ethylene-bridged nucleic acid (ENA) modification (see e.g., Koizumi (2006), “ENA oligonucleotides as therapeutics”. Current Opinion in Molecular Therapeutics. 8 (2): 144-149). Other sugar modified nucleosides include, for example, bicyclohexose nucleic acids (see e.g., WO2011/017521) or tricyclic nucleic acids (see e.g., WO2013/154798). Sugar modification also include those where the sugar moiety is replaced with a non-sugar moiety, for example in the case of peptide nucleic acids (PNA), or morpholino nucleic acids.

In some embodiments, the single or double-stranded nucleic acid of the present disclosure comprise one or more phosphate backbone modifications. In some embodiments, the phosphate backbone modification is a 5′ phosphorylation. Additional phosphate backbone modifications include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. Representative U.S. patents that teach the preparation of the above phosphorus-containing backbones include, but are not limited to, U.S. Pat. Nos. 3,687,808; 4,469,863; 4,476,301; 5,023,243; 5,177,195.

Phosphate backbone modifications may also include those that do not include a phosphorus atom, therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar modification); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones. See e.g., U.S. Pat. Nos. 5,034,506; 5,166,315; 5,185,444; 5,214,134; 5,216,141. In some embodiments, the single or double-stranded nucleic acid of the present disclosure have a morpholino backbone structure.

In some embodiments, the single or double-stranded nucleic acid of the present disclosure comprises one or more nucleic acid base modifications. Nucleic acid base modifications include, for example, the addition or substitution of a chemical group or a substitution of the nitrogen atom of the ring. Exemplary nucleic acid base modifications include but are not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleic acid base modifications include those disclosed in “Modified Nucleosides in Biochemistry”, Biotechnology and Medicine, Herdewijn, P. ed. Wiley-VCH, 2008. Some nucleic acid base modifications may be particularly useful for increasing the binding affinity of the single or double-stranded nucleic acid of the present disclosure. These may include 5-substituted pyrimidines, 6-azapyrimidines and N-2, N-6 and 0-6 substituted purines, including 2-aminopropyladenine, 5-propynyluracil and 5-propynylcytosine.

### 3. Delivery Vehicles and Pharmaceutical Compositions

### 3.1 Vectors

In some aspects, the single or double-stranded nucleic acid of the present disclosure can be delivered to a target cell by a suitable vector. Accordingly, the disclosure provides a vector comprising the single or double-stranded nucleic acid as described above. For example, the vector may comprise an antisense oligonucleotide, a ribozyme, an siRNA or shRNA, or a gRNA that target lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 AL157871.4-201, HNRNPA2/B1 or SNX10.

Vectors and methods useful for the delivery of the single or double-stranded nucleic acid are well known in the art. Generally, DNA encoding the ASO, the ribozyme, the siRNA or shRNA, or the gRNA is cloned into a vector downstream of a promoter for expression. In some embodiments, the vector is a viral vector. Exemplary viral vectors include retroviral, lentiviral, adeno-associated viral (AAV) vectors. Retroviral vectors for the delivery of nucleic acids are described e.g., in Miller et al. (1993), “Use of retroviral vectors for gene transfer and expression”, Methods Enzymol 217:581-599; Salmons and Gunzberg, (1993), Human Gene Therapy 4:129-141; and Grossman and Wilson, (1993) Curr. Opin. in Genetics and Devel. 3:110-114. Lentiviral vectors contemplated for use are described e.g., in U.S. Pat. Nos. 6,143,520; 5,665,557; and 5,981,276, which are herein incorporated by reference. Suitable AAV vectors are described e.g., in Aponte-Ubillus et al., 2018, “Molecular Design For Recombinant Adeno-Associated Virus (Raav) Vector Production” Applied microbiology and biotechnology 102.3:1045-1054; Naso et al., 2017, “Adeno-Associated Virus (Aav) As A Vector For Gene Therapy” BioDrugs 31:317; Penaud-Budloo et al., 2018., “Pharmacology of Recombinant Adeno—Associated Virus Production” Molecular Therapy: Methods & Clinical Development 8:166-180; Walsh et al., (1993) Proc. Soc. Exp. Biol. Med. 204:289-300; Samulski et al. (1987), J. Virol. 61: 3096-3101; Fisher et al. (1996), J. Virol, 70: 520-532; Samulski et al. (1989), J. Virol. 63: 3822-3826; and U.S. Pat. Nos. 5,436,146; 5,252,479; 5,139,941. Other viral vectors that may be used include, but are not limited to, adenoviruses (AV), pox viruses, alphaviruses, herpes viruses, bovine papilloma virus (BPV-I), and Epstein-Barr virus (pHEBo, pREP-derived and p205). A suitable AV vector and a method for delivering the vector into target cells, is described, for example, in Xia et al. (2002), Nat. Biotech. 20: 1006-1010.

Any suitable promoter that can direct transcription initiation of the sequences encoded by the nucleic acids may be used. The promoter may be an inducible promoters, organism specific promoters, tissue specific promoters, or a cell type specific promoter. Examples of promoters include, but are not limited to, simian virus 40 (SV40) early promoter, a mouse mammary tumour virus promoter, a human immunodeficiency virus long terminal repeat promoter, a Moloney virus promoter, an avian leukaemia virus promoter, an Epstein-Barr virus immediate early promoter, a Rous sarcoma virus (RSV) promoter, a human actin promoter, a human myosin promoter, a human haemoglobin promoter, cytomegalovirus (CMV) promoter and a human muscle creatine promoter, a metallothionine promoter, a glucocorticoid promoter, a progesterone promoter, and a tetracycline promoter (tet-on or tet-off), a HER-2 promoter, and PSA associated promoter. In some embodiments, the promoter is a U6 or H1 promoter.

The gene encoding the ASO, the ribozyme, the siRNA or shRNA, or the gRNA of the present disclosure may also be under the control of other regulatory elements such as enhancer or activator sequences, leader or signal sequences, ribosomal binding sites, transcription start and termination sequences, and polyadenylation sequence. Enhancers that may be used in approaches of the invention include but are not limited to: an SV40 enhancer, a cytomegalovirus (CMV) enhancer, an elongation factor 1 (EF1) enhancer, yeast enhancers, viral gene enhancers, and the like. Termination control region may comprise or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like. Such regulatory elements are described e.g., in Molecular Cell Biology Editors: H. Lodish et al., 8th edition 2016.

The vectors described herein may also be used to deliver CRISPR elements, including the gRNAs (e.g., sgRNAs or other gRNAs), Cas proteins (with or without nuclease activity), and Cas-transcriptional activator fusion proteins (see e.g., Byrne et al. (2014), “Genome editing in human stem cells”, Methods in Enzymology. 2014; 546:119-138; Dunbar et al., 2018, “Gene Therapy Comes Of Age” Science 359:6372; and Cong et al., Science (80). 339, 819-823).

The vectors described herein may be generated and isolated using methods known in the art. See, e.g., U.S. Pat. Nos. 7,790,449, 7,588,772, and Zolotukin et al., “Production And Purification Of Serotype 1, 2, And 5 Recombinant Adeno-Associated Viral Vectors.” Methods 28:158-167 (2002), Penaud-Budloo et al., 2018; Gongalves, M. A. “Adeno-associated virus: from defective virus to effective vector.” Virol J 2: 43 (2005); Li, et al “Engineering adeno-associated virus vectors for gene therapy.” Nat Rev Genet 21: 255-272 (2020); all incorporated by reference and cited above. For general methods on genetic and recombinant engineering, recombinant engineering, and transfection techniques see e.g., Sambrook et al, Molecular Cloning: A Laboratory Manual, Cold Spring Harbor Press, Cold Spring Harbor, N.Y.; Graham et al., Virol., 52:456 (1973); Davis et al., Basic Methods in Molecular Biology, Elsevier, (1986); and Chu et al., Gene 13:197 (1981).

Non-viral vectors or methods can also be used to deliver the nucleic acids of the present disclosure. In one approach, virus-like particles (VLP's) are used to deliver the ASO, siRNA or shRNA, the ribozyme, or the gRNA. The VLP comprises an engineered version of a viral vector, where nucleic acid cargo are packaged into VLPs through alternative mechanisms (e.g., mRNA recruitment, protein fusions, protein-protein binding). See Itaka and Kataoka, 2009, “Recent development of nonviral gene delivery systems with virus-like structures and mechanisms,” Eur J Pharma and Biopharma 71:475-483; and Keeler et al., 2017, “Gene Therapy 2017: Progress and Future Directions” Clin. Transl. Sci. (2017) 10, 242-248, incorporated by reference.

### 3.2 Pharmaceutical Compositions

Another aspect of the invention pertains to pharmaceutical compositions the single or double-stranded nucleic acid or the vector as described herein. In some embodiments, the pharmaceutical composition comprises an effective amount of the single or double-stranded nucleic acid or the vector comprising the same and a pharmaceutically acceptable carrier.

In some embodiments, the pharmaceutical composition further comprising a specific inhibitor of one or more kinases selected from the group consisting of MEK, PLK1, TAF, AURKA, HER, PTK2, PKD, PKC, IKBK, MAP3K, PIM, SRC, PAK, AKT, ERK, and RAF. Specific inhibitors of these kinases are well known in the art and include, but are not limited to trametinib, volasertib, tozasertib, alisertib, Bay-299, CeMMEC1. For example, the pharmaceutical composition may comprise an antisense oligonucleotide or a ribozyme and a specific kinase inhibitor, such as trametinib, volasertib, tozasertib, alisertib, Bay-299, and/or CeMMEC1. In another example, the pharmaceutical composition may comprise an siRNA or shRNA and a specific kinase inhibitor, such as trametinib, volasertib, tozasertib, alisertib, Bay-299, and/or CeMMEC1. In yet another example, the pharmaceutical composition may comprise a gRNA and a specific kinase inhibitor, such as trametinib, volasertib, tozasertib, alisertib, Bay-299, and/or CeMMEC1. In some embodiments, the pharmaceutical composition comprises an effective amount of the single or double-stranded nucleic acid or the vector comprising the same, an effective amount of a specific kinase inhibitor, and a pharmaceutically acceptable carrier.

A suitable pharmaceutically acceptable carrier may be buffered saline or other buffers, e.g., HEPES, to maintain pH at appropriate physiological levels, stabilizing agents, adjuvants, diluents, or surfactants. Exemplary pharmaceutically acceptable carriers include sterile, pyrogen-free water and sterile, pyrogen-free, phosphate buffered saline (PBS), sodium and potassium salts. A variety of such known carriers are provided in U.S. Patent Publication No. 7,629,322 and PCT Publication No. WO 2007/031091, incorporated herein by reference. In some embodiments, the pharmaceutically acceptable carrier is PBS. The carrier may be, for example an isotonic sodium chloride solution, or a balanced salt solution.

In some approaches, sterile injectable solutions can be prepared with the nucleic acids or the vectors in the required amount and pharmaceutically acceptable carrier or an additive suitable for injection into a human. For injection, the carrier or excipient will typically be a liquid.

In some embodiments, the pharmaceutically acceptable carrier comprises a copolymer, a lipid, or a nanoparticle. In some embodiments, the nanoparticle is a liposomal nanoparticle. Suitable pharmaceutically acceptable carrier include, for example, the cationic lipid Genzyme Lipid 67 (GL67), polyethylene glycol (PEG) liposomes, cationic liposomes, chitosan nanoparticles and cationic cell penetrating peptides (CPPs). Additional exemplary carriers and encapsulation methods that can be used are described e.g., in Ozcan et al. (2015), “Preclinical and clinical development of siRNA-based therapeutics”, Adv. Drug Deliv. Rev., 87, 108-119 and Juliano (2016), “The delivery of therapeutic oligonucleotides”, Nucleic Acids Research, 2016, Vol. 44, No. 14. In some embodiments, the nucleic acids described herein are encapsulated in liposomes. In some embodiments, the nucleic acids described herein are encapsulated in gold nanoparticles.

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

In some embodiments, the disclosure provides the use of the single or double-stranded nucleic, the vector, or the pharmaceutical composition described herein for the preparation of a medicament for treating cancer. In some embodiments, the disclosure relates to the single or double-stranded nucleic, the vector, or the pharmaceutical composition as described herein for the preparation of a medicament for treating cancer.

### 4. Administration and Dosage

### 4.1 Administration

Aspects of the invention include methods of administering a therapeutically-effective amount of the single or double-stranded nucleic acid and/or specific kinase inhibitor to a subject. In one embodiment, the subject is a human. Administration is not limited to a particular site or method. Any suitable route of administration or combination of different routes can be used, including topical (such as, to the skin) or enteral (such as, orally or through the gastrointestinal tract) or systemic administration (e.g., intravenous, intravascular, intraarterial), or local injection (intratumoral, intraocular, intramuscular, subcutaneous, intradermal injection, transdermal, intracranial, intracerebral, intracerebroventricular, or intrathecal injection). In some embodiments, the nucleic acids, specific kinase inhibitors, or pharmaceutical compositions are administered through subcutaneous intratumoral injections.

Administration can be performed by use of an osmotic pump, by electroporation, or by other means. In some approaches, administration of the nucleic acid, specific kinase inhibitor, or pharmaceutical compositions can be performed before, after, or simultaneously with surgical tumor removal or biopsy.

### 4.2 Dosage and Effective Amounts

Dosage values may depend on the nature of the product and the severity of the condition. It is to be understood that for any particular subject, specific dosage regimens can be adjusted over time and in course of the treatment according to the individual need and the professional judgment of the person administering or supervising the administration of the compositions. Accordingly, dosage ranges set forth herein are exemplary only and are not intended to limit the scope or practice of the claimed composition.

The amount of the nucleic acids and/or specific kinase inhibitors administered will be an “effective amount” or a “therapeutically effective amount,” i.e., an amount that is effective, at dosages and for periods of time necessary, to achieve a desired result. A desired result would include inhibition of expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 AL157871.4-201, HNRNPA2/B1 or SNX10, inhibition of a cancer cell (e.g., a NRAS-mutated cancer cell or a BRAF-mutated cancer cell), reduction in tumor size and/or tumor growth, prolonged survival or a detectable improvement in a symptom associated with cancer that improves patient quality of life. Alternatively, if the pharmaceutical composition is used prophylactically, a desired result would include a demonstrable prevention of one or more symptoms of cancer. A therapeutically effective amount of such a composition may vary according to factors such as the disease state, molecular tumor profile (e.g. tumor mutation types), age, sex, and weight of the individual, or the ability of the nucleic acid and/or kinase inhibitor to elicit a desired response in the individual. Dosage regimens may be adjusted to provide the optimum response. A therapeutically effective amount is also one in which any toxic or detrimental effects of the nucleic acid and/or kinase inhibitor are outweighed by the therapeutically beneficial effects.

Generally, nucleic acids of the present invention, such as an antisense oligonucleotide, siRNA or shRNA, ribozyme, or gRNA may be administered less than 75 mg per kg of body weight, such as for example less than 70, 60, 50, 40, 30, 20, 10, 5, 2, 1, 0.5, 0.1, 0.05, 0.01, 0.005, 0.001, or 0.0005 mg per kg of body weight. Exemplary dosage ranges for kinase inhibitors may be 5-100 mg/kg/week, depending on the inhibitor. As non-limiting examples, in some embodiments, trametinib is administered at 2 mg/kg/day and/or volasertib is administered at 50 mg/kg/week (both oral gavage). This refers to oral gavage, other routes may require other forms of dosage and application frequency. The particular amounts may be determined by conventional tests which are well known to the person skilled in the art. Suitable tests are, for example, described in Tamhane and Logan (2002), “Multiple Test Procedures for Identifying the Minimum Effective and Maximum Safe Doses of a Drug”, Journal of the American statistical association, 97(457):1-9. If a vector is used as a delivery system, quantification of genome copies (GC), vector genomes (VG), virus particles (VP), or infectious viral titer may be used as a measure of the dose contained in a formulation or suspension. Any method known in the art can be used to determine the GC, VG, VP or infectious viral titer as described in, e.g. in Dobkin et al., “Accurate Quantification and Characterization of Adeno-Associated Viral Vectors.” Front Microbiol 10: 1570-1583 (2019); Lock et al., “Absolute determination of single-stranded and self-complementary adeno-associated viral vector genome titers by droplet digital PCR.” Hum Gene Ther Methods 25: 115-125 (2014); and Grimm, et al. “Titration of AAV-2 particles via a novel capsid ELISA: packaging of genomes can limit production of recombinant AAV-2.” Gene Ther 6: 1322-1330 (1999); which are incorporated herein by reference. An exemplary human dosage range in vector genomes per kilogram bodyweight (vg/kg) may be 10e6 vg/kg-10e15/kg vg per injection in a volume of 1-100,000 pl.

In one approach, the nucleic acid and/or specific kinase inhibitor, or pharmaceutical composition is administered in a single dosage. In another embodiment, the method involves administering the compositions in two or more dosages (e.g., split dosages). In another embodiment, the composition is administered at different locations. In another embodiment, a second administration is performed at a later time point. Such time point may be weeks, months or years following the first administration. In some embodiments, multiple treatments may be required in any given subject over a lifetime.

### 4.3 Combination Therapies

In some approaches, the nucleic acids and/or kinase inhibitors of the present disclosure are used in combination with one or more additional anti-cancer agents and/or therapies, including any known, or as yet unknown, anti-cancer agent or therapy which helps preventing development of, slowing progression of, reversing, or ameliorating the symptoms of cancer. The one or more additional anti-cancer agents and/or therapies may be administered and/or performed before, concurrent with, or after administration of the nucleic acids described herein. The combined administration includes co-administration, using separate formulations or a single pharmaceutical formulation. In some embodiments, the nucleic acids of the present disclosure are used in combination with one or more anticancer therapies, such as chemotherapy, radiation therapy, immunotherapy, and surgical treatment.

In one embodiment, the nucleic acids and/or kinase inhibitors are used in combination with other kinase inhibitors. Exemplary kinase inhibitors include, but are not limited to trametinib or volasertib or both.

Other chemotherapeutic agents that may be used in combination with the nucleic acids and/or kinase inhibitors include temozolomide (TMZ), cyclophosphamide, docetaxel, hydroxydaunorubicin, adriamycin, doxorubicin, vincristine, and prednisolone.

In some approaches, the nucleic acid and/or kinase inhibitors of the present disclosure are used in combination with immunotherapy, for example a checkpoint inhibitor, such as ipilimumab, nivolumab, pembrolizumab, atezolizumab, avelumab, or durvalumab.

Examples of other anti-cancer agents that can be combined with the nucleic acids and or kinase inhibitors includes, without limitation any one or more of a co-stimulation molecule blocker, an adhesion molecule blocker, an antiangiogenic agent (e.g., bevacizumab), an anti-cytokine antibody or functional fragment thereof, a corticosteroid, a non-steroidal anti-inflammatory agent, a nitrogen mustard, an aziridine, an alkyl sulfonate, a nitrosourea (e.g., carmustine, semustine, lomustine, nimustine, or fotemustine), a non-classical alkylating agent, a folate analog, a purine analog, an adenosine analog, a pyrimidine analog, a substituted urea, an antitumor antibiotic, an epipodophyllotoxin, a microtubule agent, a camptothecin analog, a cytokine, a monoclonal antibody, a recombinant toxin, an immunotoxin, a cancer gene therapy, a cancer cell therapy, an oncolytic viral therapy, or a cancer vaccine.

### 5. Method of Treating Cancer

In some aspects, the present disclosure provides a method of inhibiting a cancer cell. The method comprises contacting the single or double-stranded (e.g., the ASO, the ribozyme, the siRNA or shRNA, or the gRNA), the vector, or the pharmaceutical composition comprising the same with the cancer cell such that expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 AL157871.4-201, HNRNPA2/B1 or SNX10 is inhibited.

In some aspects, the method further comprises contacting the cancer cell with a specific inhibitor of MEK, PLK1, TAF, AURKA, HER, PTK2, PKD, PKC, IKBK, MAP3K, PIM, SRC, PAK, AKT, ERK, or RAF. In some embodiments, the specific inhibitor is trametinib, volasertib, tozasertib, alisertib, Bay-299, CeMMEC1. In some approaches, the cancer cell may be contacted with two or more specific inhibitor of MEK, PLK1, TAF, AURKA, HER, PTK2, PKD, PKC, IKBK, MAP3K, PIM, SRC, PAK, AKT, ERK, or RAF.

The cancer cell may be contacted with s specific kinase inhibitor only. In some approaches, a cancer cell may be inhibited by contacting the cancer cell with a specific inhibitor alone without using any of the nucleic acids described above. Accordingly, in some aspects, the present disclosure provides a method of inhibiting a cancer cell, where the cancer cell is contacted with a specific inhibitor of MEK, PLK1, TAF, AURKA, HER, PTK2, PKD, PKC, IKBK, MAP3K, PIM, SRC, PAK, AKT, ERK, or RAF in an amount to inhibit the cancer cell growth. In some embodiments, the specific inhibitor is trametinib, volasertib, tozasertib, alisertib, Bay-299, CeMMEC1. In some approaches, the cancer cell may be contacted with two or more specific inhibitors of MEK, PLK1, TAF, AURKA, HER, PTK2, PKD, PKC, IKBK, MAP3K, PIM, SRC, PAK, AKT, ERK, or RAF.

In some embodiments, the cancer cell is a NRAS-mutated cancer cell. In some aspects, the NRAS-mutated cancer cell comprises a NRAS G12A, NRAS G12C, NRAS G12D, NRAS G12R, NRAS G12S, NRAS G12V, NRAS G13D, NRAS G12S2, NRAS G13A, NRAS G13S, NRAS G13V, NRAS G13R, NRAS G13C, NRAS Q61H, NRAS Q61L, NRAS Q61R, NRAS A146T, or a NRAS A146V mutation.

In some embodiments, the cancer cell is a BRAF-mutated cancer cell. In one aspect, the BRAF-mutated cancer cell comprises a BRAF V600E mutation. In some aspects, the BRAF-mutated cancer cell comprises a BRAF R461I, BRAF I462S, BRAF G463E, BRAF G463V, BRAF G465A, BRAF G465E, BRAF G465V, BRAF G468A, BRAF G468E, BRAF N580S, BRAF E585K, BRAF D593V, BRAF F594L, BRAF G595R, BRAF L596V, BRAF T598I, BRAF V599D, BRAF V599E, BRAF V599K, BRAF V599R, BRAF V600K, or a BRAF A727V mutation. Other BRAF mutations are described e.g., in Davies at al. (2002), “Mutations of the BRAF gene in human cancer”, Nature, 27; 417(6892):949-54; and Dankner et al. (2018), Classifying BRAF alterations in cancer: new rational therapeutic strategies for actionable mutations. Oncogene, 37(24):3183-3199.

### 5.1 Patients

In some embodiments, the cancer cell that is contacted with the nucleic acid of the present disclosure and/or with a specific kinase inhibitor is in a mammal, such as a human, a non-human primate, a mouse, a dog, a cat, a horse, a rabbit, a cow, a pig, or a sheep. In some embodiments, the cancer cell that is contacted with the nucleic acid of the present disclosure and/or with a specific kinase inhibitor is in a human. In some embodiments, the human is receiving a treatment and the treatment involves contacting the human cell with the nucleic acid of the present disclosure and/or with a specific kinase inhibitor. Humans who are candidates for treatment with the nucleic acid and/or with a specific kinase inhibitor include “patients” or “subjects” experiencing or having experienced one or more signs, symptoms, or other indicators of cancer.

In some approaches, patients are selected for treatment based on signs, symptoms, clinical phenotypes and/or biomarkers. In some embodiments, they may be assessed via a clinical exam, including but not limited to imaging and morphological assessments, such as magnetic resonance imaging (MRI), biopsy, or bloodwork for the detection of circulating tumor cells or cell-free DNA from tumor cells.

In some aspects, patients receiving therapy with the nucleic acid and/or with a specific kinase inhibitor may include those which have previously not responded to conventional anti-cancer treatment, such as chemotherapy or radiotherapy. In certain aspects, patients receiving therapy with the nucleic acid and/or with a specific kinase inhibitor may include those which have previously not responded to a kinase inhibitor treatment. In some embodiments, the patient has not responded to a treatment involving a MEK inhibitor, a MAPK inhibitor, and/or a BRAF, and/or any other kinase inhibitor. In some aspects, patients include those that show resistance to a kinase inhibitor treatment. In some aspects, patients include those that show resistance to a MEK inhibitor treatment, a MAPK inhibitor treatment, and/or a BRAF inhibitor treatment. In some aspects, the resistance is an acquired resistance. In some aspects, the resistance is an intrinsic resistance.

In certain embodiments, patients receiving therapy with the nucleic acid and/or with a specific kinase inhibitor may include those which have newly diagnosed cancer. In some embodiments, the cancer treated with the nucleic acid and/or with a specific kinase inhibitor described herein is recurrent cancer. In another embodiment, the cancer is recurrent skin cancer.

In one aspect, administration of the nucleic acids and/or the specific kinase inhibitor is performed at a very early stage disease progression may provide superior therapeutic benefit. For example, treatment may be performed prior to the appearance of signs or symptoms of cancer. Thus, provided herein are methods and compositions for preventing development of cancer. In some approaches, the patient has no symptoms of cancer.

In some approaches, patients are assessed by genotyping to determine their individual genetics (e.g., by assessing the presence of risk alleles associated with one or more cancers described below) and associated risk of disease. In some embodiments, patients include those that carry a NRAS-mutation. In some embodiments, patients disclose those who carry a BRAF-mutation. Accordingly, in some approaches, at the time of first administration of the composition, the patient does not exhibit any of the clinical phenotypes of cancer.

### 5.2 Cancers

The compositions and methods described herein find particular use for treatment of patients or subjects with, or at risk of developing, cancer. Examples of cancers include solid cancers and sarcomas, such as skin cancer, melanoma, liver cancer, brain cancer, head and neck cancer, stomach cancer, lung cancer, breast cancer, uterine cancer, ovarian cancer, hepatic cancer, bronchial cancer, epipharynx carcinoma, pharyngeal cancer, esophageal cancer, bladder cancer, pancreatic cancer, prostate cancer, colon cancer, osteosarcoma, thyroid cancer, parathyroid cancer, ureteral cancer and cervical cancer, and malignant tumors formed in hemopoietic organs or blood, e.g. leukemia such as acute lymphatic leukemia, malignant lymphoma. In some embodiments, the cancer is skin cancer. In some embodiments, the skin cancer is melanoma. Other examples of cancers affecting the skin include basal cell carcinoma and squamous cell carcinoma.

Accordingly, in some embodiments, the cancer cell that is contacted with the nucleic acid of the present disclosure and/or with a specific kinase inhibitor is a melanoma cell. In some embodiments, the cancer cell is a metastatic melanoma cancer cell. In certain embodiments, the cancer cell is a MEK-therapy resistant cancer cell. In some embodiments, the cancer cell is a MAPK-therapy resistant cancer cell. In some embodiments, the cancer cell is a BRAF-therapy resistant cancer cell.

### 6. Summary of Sequences

LncRNA Nr. 1:
Gene name: BX470102.3
Genecode ID: ENSG00000238279.1 for the gene, ENST00000420695.1
for the trancript Chromosome: 1
Strand: +
Mature mRNA length: 531nt
Predicted mature mRNA:
>ENST00000420695.1
(SEQ ID NO: 1)
Ccccacccagtggggacagaagacaacttaattccacaaagttggacccccaagggaagtggggaggtgagagggaa
agaaggatgtggtctccatctaaagtcaagatctcttccccagaagctagaggtaaacttgcccagctcggactagg
cctagagaggctgaatgatgtggcatcacccggaaacagcgtttaccctccttatcctcttcccttctgcctgaaaa
cactaatccagatgatggacaatgattcaattagtcatcttctctgatgggggctgagatccaggctgggatcccgt
ggaagtgccgggcaatcctctctgcaagtggctctgtgctccctcatcaccaaggacccatgtcactttggcattgc
ttctcctcagctacttctcagttactggtcctcatttggagagatggatatccggctggaagcatcccctacccgct
gggagagtgggtctacagctcagggtctacatgtggaccagggcctcagaatgtgggtaaatgtgagtc
Antisense oligonucleotide sequences to target BX:
1:
((SEQ ID NO : 13)
AAGGAGGGTAAACGCT
2:
(SEQ ID NO: 14)
ATCATCTGGATTAGTG
siRNA sequence to target BX:
Sense:
(SEQ ID NO: 23)
CCCAGAAGCUAGAGGUAAAUU
Antisense:
(SEQ ID NO: 24)
UUUACCUCUAGCUUCUGGGUU
LncRNA Nr. 2:
Gene name: AC004540.4
Genecode ID: ENSG00000225792 for the gene
Chromosome: 7
Strand: −
Isoform 1:
Geneocode ID: ENST00000451368
RefSeq ID: NR 136271.1
Mature mRNA length: 611nt
Predicted mature mRNA:
>ENST00000451368.1
(SEQ ID NO: 2)
Agcccagtcgccgccgccagcggagcggcccgggcgggacgcggcgggagcgcgcgtgtgcgggacgcagcgcgggg
gatgcgcgcgggccgcggaggcgccgcaaccaacaggcggccgagggtgcagccgcgggagcgccaccgccagtggg
gtggggggcaaagctataaagaaggcccagaggattcctgcggagctgtatttactttacatccatgtgaactgctg
tcatcactactgtgtccaagcccagaggatgaactggaaaagaagagagggggaaaataataaaaagaggaaattgg
ttttcacaacacactcaaagcctgagtaacagaggagaactttaattatctccagtcacaaagagagacaggaaatt
tggacttttaattagccatttggagtgcagttgggtatttttttagctagataatttaaacgcgaataattcaagtc
tgactaaatgaaagtcacataatcagaatgcaaataattgaatttctactgcattcattaattcagtgtggaggtgt
gtgtgaagactactatgatgagctgtcacagctcaataaaatctcagtcaattaattttttcattatcttag
Isoform 2:
Genecode ID: ENST00000451264
RefSeq ID: NR_136270.1
Mature mRNA length: 508nt
Predicted mature mRNA:
>ENST00000451264.1
(SEQ ID NO: 3)
Ccgcccctcaaccaacccccaggcggcaaggccctctccacgcgcgctctccagcctggcggggccctggaagccgg
gacacgcggaggccgggagggtcatcggcgtttaaggcagcctcccacaccaagtgcaccgcccggatcccctctgc
acgagggctttctgcttattgctcttttccccagcagccagaatcgtcaccgtagcgcgggaaggggcctcgcgggc
cgtctgcagcaggtgcggccgggagccgcaggcccgcgcggattcctgcggagctgtatttactttacatccatgtg
aactgctgtcatcactactgtgtccaagcccagaggatgaactggaaaagaagagagggggaaaataataaaaagag
gaaattggttttcacaacacactcaaagcctgagtaacagaggagaactttaattatctccagtcacaaagagagac
aggaaatttggacttttaattagccatttggagtgcagttgggtat
Antisense oligonucleotide sequences to target AC004540.4:
1:
(targets exon region of both isoforms)
(SEQ ID NO: 15)
GACTGGAGATAATTAA
2:
(targets intronic region of Isoform 2)
(SEQ ID NO: 16)
TGCGCGGCGGAAAGAA
siRNA Sequence to target both Isoforms of AC004540.4:
Sense:
(SEQ ID NO: 25)
GAGUAACAGAGGAGAACUUUU
Antisense:
(SEQ ID NO: 26)
5′-PAAGUUCUCCUCUGUUACUCUU
IncRNA Nr.3
Gene name: RP11-7011.3
Genecode ID: ENSG00000237950.1 for the gene
Chromosome: 1
Strand: −
Isoform 1:
Genecode ID: ENST00000446167.1
Mature mRNA length: 486nt
Predicted mature mRNA:
>ENST00000446167.1
(SEQ ID NO: 4)
gggaccacccaataagcaaccgggaaaccacataccatggacagtgctcggggaatttcggctcctcctcatccctg
gaggttccagggatctggcagagaaagaagcccagtctcacagcaatgactcctgaacatcctttgcaaacactatt
taggacagatggactgaagtaggccctggggagagttgggggcatttccggaggccagaccaaaggtgatctccagg
agatttggatgttagtgcaggatagctcagagccaagtggcagataagtctttgggagccagaagggctctttcttc
tctaaggaacaagtgtagacctataggggcaagaacctgtggcaaccctgggagaggtctgcagattcaggagggaa
catcccaaaactcagcagaggaggagagggagcttttgttggtcacctggaaccactaccaatccatgttcatgtca
aattaaatgatcactttgaagttt
Isoform 2:
Genecode ID: ENST00000445226.1
Mature mRNA length: 294nt
Predicted mature mRNA:
>ENST00000445226.1
(SEQ ID NO: 5)
caaccgggaaaccacataccatggacagtgctcggggaatttcggctcctcctcatccctggaggttccagggatct
ggcagagaaagaagcccagtctcacagcaatgactcctgaacatcctttgcaaacactatttaggacagatggactg
aagtaggccctggggagagttgggggcatttccggagggaacatcccaaaactcagcagaggaggagagggagcttt
tgttggtcacctggaaccactaccaatccatgttcatgtcaaattaaatgatcactttgaagt
Isoform 3:
Genecode ID: ENST00000412378.1
Mature mRNA length: 494nt
Predicted mature mRNA:
>ENST00000412378.1
(SEQ ID NO: 6)
cagaccggcgcggggcgctgcggcccaactccttagtaggacgacgtgactcgaggggccggaggacggagggctcc
tcctcatccctggaggttccagggatctggcagagaaagaagcccagtctcacagcaatgactcctgaacatccttt
gcaaacactatttaggacagatggactgaagtaggccctggggagagttgggggcatttccggaggccagaccaaag
gtgatctccaggagatttggatgttagtgcaggatagctcagagccaagtggcagataagtctttgggagccagaag
ggctctttcttctctaaggaacaagtgtagacctataggggcaagaacctgtggcaaccctgggagaggtctgcaga
ttcaggagggaacatcccaaaactcagcagaggaggagagggagcttttgttggtcacctggaaccactaccaatcc
atgttcatgtcaaattaaatgatcactttgaa
Antisense oligonucleotide sequences to target RP11-7011.3:
1:
[targeting SEQ ID NOS: 4, 5 and 6]
(SEQ ID NO: 17)
ACATGGATTGGTAGTG
2:
[targeting SEQ ID NOS: 4, 5 and 6]
(SEQ ID NO: 18)
GATCATTTAATTTGAC
siRNA Sequence to target both Isoforms of RP11-7011.3:
Sense:
[targeting SEQ ID NOS: 4, 5 and 6]
(SEQ ID NO: 27)
AGCAAUGACUCCUGAACAUUU
Antisense:
(SEQ ID NO: 28)
5′-PAUGUUCAGGAGUCAUUGCUUU
IncRNA Nr.4
Gene name: RN7SL1
Genecode ID: ENSG00000258486.1 for the gene
Chromosome: 14
Strand: +
Isoform 1:
Genecode ID: ENST00000635274.1
Mature mRNA length: 300nt
Predicted mature mRNA:
>ENST00000635274.1
(SEQ ID NO: 7)
Cgccgggcgcggtggcgcgtgcctgtagtcccagctactcgggaggctgaggctggaggatcgcttgagtccaggag
ttctgggctgtagtgcgctatgccgatcgggtgtccgcactaagttcggcatcaatatggtgacctcccgggagcgg
gggaccaccaggttgcctaaggaggggtgaaccggcccaggtcggaaacggagcaggtcaaaactcccgtgctgatc
agtagtgggatcgcgcctgtgaatagccactgcactccagcctgggcaacatagcgagaccccgtctct
Isoform 2:
Genecode ID:
ENST00000618786.1
Mature mRNA length: 300nt
Predicted mature mRNA: 299
>ENST00000618786.1
(SEQ ID NO: 8)
gccgggcgcggtggcgcgtgcctgtagtcccagctactcgggaggctgaggctggaggatcgcttgagtccaggagt
tctgggctgtagtgcgctatgccgatcgggtgtccgcactaagttcggcatcaatatggtgacctcccgggagcggg
ggaccaccaggttgcctaaggaggggtgaaccggcccaggtcggaaacggagcaggtcaaaactcccgtgctgatca
gtagtgggatcgcgcctgtgaatagccactgcactccagcctgggcaacatagcgagaccccgtctct
siRNA Sequence to target:
Sense:
[targeting SEQ ID NOS: 7 and 8]
(SEQ ID NO: 29)
GCACUAAGUUCGGCAUCAAUU
Antisense:
(SEQ ID NO: 30)
UUGAUGCCGAACUUAGUGC
2:
Sense:
[targeting SEQ ID NOS: 7 and 8]
(SEQ ID NO: 31)
ACUAAGUUCGGCAUCAAUAUU
Antisense:
(SEQ ID NO: 32)
UAUUGAUGCCGAACUUAGU
3:
Sense:
[targeting SEQ ID NOS: 7 and 8]
(SEQ ID NO: 33)
GGACCACCAGGUUGCCUAAUU
Antisense:
(SEQ ID NO: 34)
UUAGGCAACCUGGUGGUCC
4:
Sense:
[targeting SEQ ID NOS: 7 and 8]
(SEQ ID NO: 35)
GGGACCACCAGGUUGCCUAUU
Antisense:
(SEQ ID NO: 36)
UAGGCAACCUGGUGGUCCC
IncRNA Nr.5
Gene name: ARF-AS1
Genecode ID: ENSG00000272146 for the gene
Chromosome: 3
Strand: +
Isoform 1:
Genecode ID: ENST00000606192.5
Mature mRNA length: 327nt
Predicted mature mRNA:
>ENST00000606192.5
(SEQ ID NO: 9)
cttgcttccggaaaggcgagctgagcattatgggttagggtctcactttgtcacccaagctgaagtacagtggcatc
atctcggcttactcaacctcctgggatcaagtgatcctcccacctcagcccccaaagtagctgggactacaggtcag
gcatggtggctcacacctgtaatcccagcatgttgggaggccaagatgggagactcacttgagcccagaagttccag
accagccttggcaatatagtgagatgccatttctattttaaaaaatattttaaaaataaaatatttttctattcacc
tttcatcaatacaaaccca
Isoform 2:
Genecode ID: ENST00000607297.1
Mature mRNA length: 437nt
Predicted mature mRNA:
>ENST00000607297.1
(SEQ ID NO: 10)
Gatgggtattccctgatgccatgaacttacacgtttcacacacgggaccagacgcttgctttagttgacgcatgaag
accggtccggtcttttgcggagaaaagtggttaaaagctgacttgtgggccgagaaactgtggcaccctaatgagct
agggctagacgcttcgaccaccacgccaagtgattctgaagatctctaattctgtcaaggcgagagcgctccaacac
gtgttcatcggctgttgctttttaaagagaaggtcaggcatggtggctcacacctgtaatcccagcatgttgggagg
ccaagatgggagactcacttgagcccagaagttccagaccagccttggcaatatagtgagatgccatttctatttta
aaaaatattttaaaaataaaatatttttctattcacctttcatcaatacaaa
Isoform 3:
Genecode ID: ENST00000607782.1
Mature mRNA length: 552nt
Predicted mature mRNA:
>ENST00000607782.1
(SEQ ID NO: 11)
attccctgatgccatgaacttacacgtttcacacacgggaccagacgcttgctttagttgacgcatgaagaccggtc
cggtcttttgcggagaaaagtggttaaaagctgacttgtgggccgagaaactgtggcaccctaatgagctagggcta
gacgcttcgaccaccacgccaagtgattctgaagatctctaattctgtcaaggcgagagcgctccaacacgtgttca
tcggctgttgctttttaaagagaagggtctcactttgtcacccaagctgaagtacagtggcatcatctcggcttact
caacctcctgggatcaagtgatcctcccacctcagcccccaaagtagctgggactacaggtcaggcatggtggctca
cacctgtaatcccagcatgttgggaggccaagatgggagactcacttgagcccagaagttccagaccagccttggca
atatagtgagatgccatttctattttaaaaaatattttaaaaataaaatatttttctattcacctttcatcaataca
aacccagaagaga
ASO Sequence to target all 3 isoforms:
(SEQ ID NO: 19)
ATTGATGAAAGGTGAA
ASO sequence targets exonic region of isoform 2 and
3 and intronic region of isoform 1:
GCGTCAACTAAAGCAA
SIRNA Sequence:
Sense:
[targeting SEQ ID NO: 9]
(SEQ ID NO: 37)
GGAAAGGCGAGCUGAGCAUUU
Antisense:
(SEQ ID NO: 38)
AUGCUCAGCUCGCCUUUCCUU
IncRNA Nr.6
Gene name: AL157871.4
Genecode ID: ENSG00000258666 for the gene
Chromosome: 14
Strand: +
Isoform 1:
Genecode ID: ENST00000557226.1
Mature mRNA length: 385nt
Predicted mature mRNA:
>ENST00000557226.1
(SEQ ID NO: 12)
Caggagccaaggaagtttatttactctactgggtgacaggagggcagagtgctccagaggagacccagatacatcaa
ccaaggacttccctgagatttggctttgctcttccagggtcaggtccttccacagatacttctcgtcatccgtcatc
tggatgaccaagggcacgttaaatacatcctggagccacctaaagaaacacagggggagaaagctgacgtctcatct
cccctgtggaggaacgccatcgtgcatctgaaaacacagctcctacttacaacgtatgttaaaacttccttgcctac
aaaatcacaatttgaattgtgatatgaatttgactatggataatgataaggtctactaccatttatcgaccaggtgg
ASO Sequence:
1.
(SEQ ID NO: 21)
AAGTCCTTGGTTGATG
2.
(SEQ ID NO: 22)
GTAAGTAGGAGCTGTG
siRNA Sequence:
Sense:
(SEQ ID NO: 39)
UGGAUAAUGAUAAGGUCUAUU
Antisense:
(SEQ ID NO: 40)
UAGACCUUAUCAUUAUCCAUU

| SEQ | Description |
|---|---|
| 1 | BX470102.3-008 |
| 2 | AC004540.4-001 |
| 3 | AC004540.4-002 |
| 4 | RP11-7011.3-001 |
| 5 | RP11-7011.3-003 |
| 6 | RP11-7011.3-002 |
| 7 | RN7SL1-202 |
| 8 | RN7SL1-201 |
| 9 | ARF-AS1-201 |
| 10 | ARF-AS1-202 |
| 11 | ARF-AS1-203 |
| 12 | AL157871.4-201 |
| 13 | Antisense oligonucleotide sequence targeting BX470102.3-008 |
| 14 | Antisense oligonucleotide sequence targeting BX470102.3-008 |
| 15 | Antisense oligonucleotide sequence targeting AC004540.4-001 |
| | and AC004540.4-002 |
| 16 | Antisense oligonucleotide sequence targeting AC004540.4-001 |
| | and AC004540.4-002 |
| 17 | Antisense oligonucleotide sequence targeting RP11-7011.3-001, |
| | 002 and 003 |
| 18 | Antisense oligonucleotide sequence targeting RP11-7011.3-001, |
| | 002, and 003 |
| 19 | Antisense oligonucleotide sequence targeting ARF-AS1-201, |
| | ARF-AS1-202, and ARF-AS1-203 |
| 20 | Antisense oligonucleotide sequence targeting ARF-AS1-202 |
| | and ARF-AS1-203 |
| 21 | Antisense oligonucleotide sequence targeting AL157871.4-201 |
| 22 | Antisense oligonucleotide sequence targeting AL157871.4-201 |
| 23 | siRNA sense strand sequence targeting BX470102.3-008 |
| 24 | siRNA antisense strand sequence targeting BX470102.3-008 |
| 25 | siRNA sense strand sequence targeting AC004540.4-001 and |
| | AC004540.4-002 |
| 26 | siRNA antisense strand sequence targeting AC004540.4-001 |
| | and AC004540.4-002 |
| 27 | siRNA sense strand sequence targeting both isoforms of |
| | RP11-7011.3-001, 002 and 003 |
| 28 | siRNA antisense strand sequence targeting both isoforms of |
| | RP11-7011.3-001, 002 and 003 |
| 29 | siRNA sense strand sequence targeting RN7SL1-201 and 202 |
| 30 | siRNA antisense strand sequence targeting RN7SL1 |
| 31 | siRNA sense strand sequence targeting RN7SL1 |
| 32 | siRNA antisense strand sequence targeting RN7SL1 |
| 33 | siRNA sense strand sequence targeting RN7SL1 |
| 34 | siRNA antisense strand sequence targeting RN7SL1 |
| 35 | siRNA sense strand sequence targeting RN7SL1 |
| 36 | siRNA antisense strand sequence targeting RN7SL1 |
| 37 | siRNA sense strand sequence targeting ARF-AS1 |
| 38 | siRNA antisense strand sequence targeting ARF-AS1 isoform 1 |
| | (SEQ ID NO: 9) |
| 39 | siRNA sense strand sequence targeting AL157871.4-201 |
| 40 | siRNA antisense strand sequence targeting AL157871.4-201 |
| 41 | Antisense oligonucleotide sequence CTCATGAGCTGTCGTA targeting |
| | AC004540.4-001 and AC004540.4-002 |
| 42 and | Duplex Sequences: 5′-GGUAAAAACAUGAAGCUAAUAGUUA-3′ and 3′- |
| 50 | ACCCAUUUUUGUACUUCGAUUAUCAAU-5′ targeting AC004540.4-001 and |
| | AC004540.4-002 |
| 43 and | Duplex Sequences: 5′-GCCAACAGCAUGUCAAUUCAGUGAT-3′ and 3′- |
| 51 | GACGGUUGUCGUACAGUUAAGUCACUA-5′, targeting AC004540.4-001 and |
| | AC004540.4-002 |
| 44 and | Duplex Sequences: 5′-AUUUCAUGUCUGAAGCAAUUCUACT-3′ and 3′- |
| 52 | AAUAAAGUACAGACUUCGUUAAGAUGA-5′, targeting AC004540.4-001 and |
| | AC004540.4-002 |
| 45 and | Duplex Sequences: 5′-ACAAAGAGAGACAGGAAAUUU-3′ and 3′- |
| 53 | AUUUCCUGUCUCUCUUUGUUU-5′, targeting AC004540.4-001 and |
| | AC004540.4-002 |
| 46 and | Duplex Sequences: 5′-UCACAAAGAGAGACAGGAAUU-3′ and 3′- |
| 54 | UUCCUGUCUCUCUUUGUGAUU-5′, targeting AC004540.4-001 and |
| | AC004540.4-002 |
| 47 | Antisense oligonucleotide sequence GACTGGAGATAATTAA- |
| | Cholesterol targeting AC004540.4-001 and AC004540.4-002 |
| 48 | Antisense oligonucleotide sequence GACCGTAGTTAGAAGG |
| | targeting HNRNPA2/B1 |
| 49 | Antisense oligonucleotide sequence AGATGGCTCTGTAAGA |
| | targeting SNX10 |

### EXAMPLE

### Example 1

A goal of this work was to explore lncRNAs interacting with the MAPK pathway that are essential for melanoma cell survival and tumor progression. As a result, we identified the oncogenic features of the lncRNA TRASH and the dependency of melanoma to TRASH expression. We suggest that the direct physical interaction of TRASH and hnRNPA2/B1 mediates the oncogenic character of TRASH. Antisense Oligonucleotide mediated TRASH knockdown (TRASHi) leads to concomitant hnRNPA2/B1 knockdown. We found that TRASH prevents apoptosis, which sustain cancer cells' viability. TRASHi efficiently suppresses these anti-apoptotic mechanisms and strongly affects a broad panel of melanoma cell lines, including melanoma that is treatment resistant to the first-line clinical approach of MEK inhibition. [Grimaldi, A. M. et al. MEK Inhibitors in the Treatment of Metastatic Melanoma and Solid Tumors. Am J Clin Dermatol 18, 745-754 (2017)] Furthermore, TRASHi leads to strong tumor growth reduction and apoptosis induction in mouse models of standard melanoma cell line xenografts and patient derived tumors. In summary, these findings demonstrate the strong potential of clinical applications of TRASHi.

### Results:

Identification of MAPK-Pathway Activation Responsive lncRNAs in Melanoma

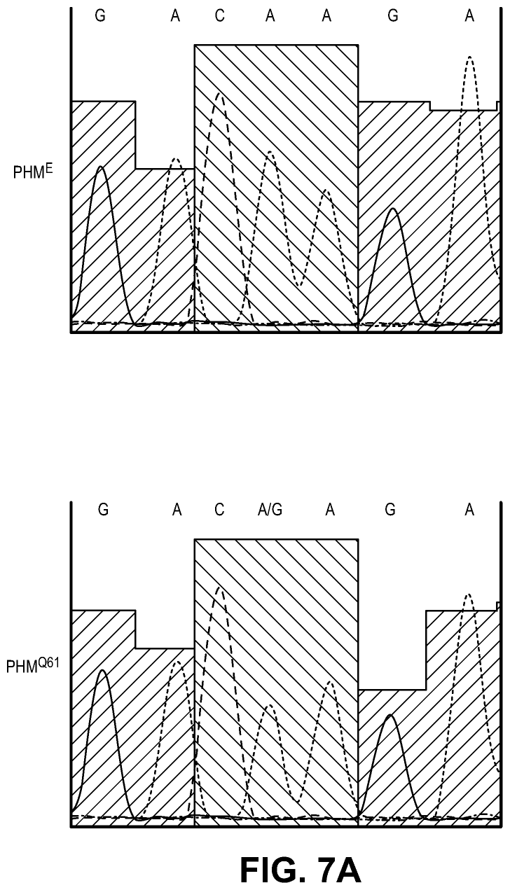

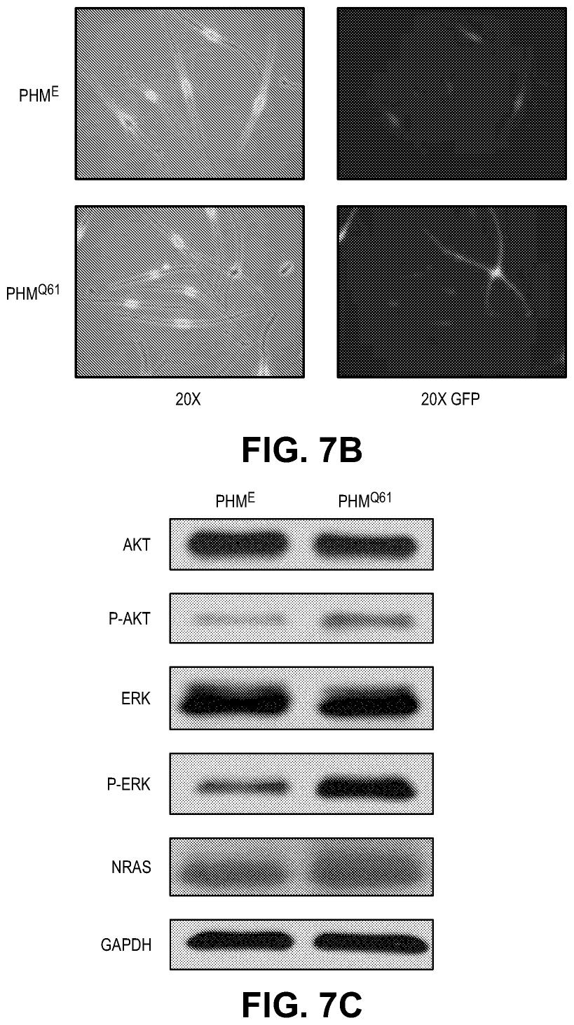

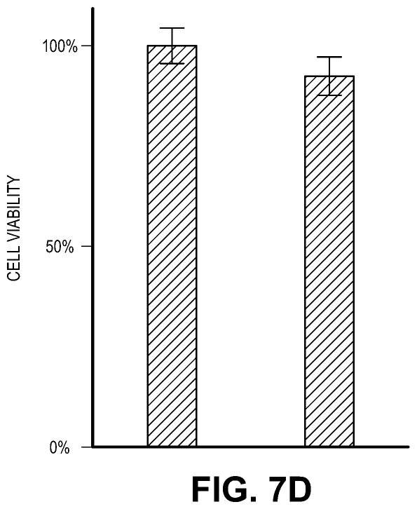

The oncogene NRAS is the most upstream member of the MAPK pathway. NRAS mutations seem to be anearly event in melanocytic tumorigenesis and NRAS activation is followed by activation of the downstream targets AKT and ERK. [Khosravi-Far, et al., Increasing Complexity of Ras Signal Transduction: Involvement of Rho Family Proteins. in Advances in Cancer Research vol. 72 57-107 (Elsevier, 1997).; Brazil, et al. Ten years of protein kinase B signalling: a hard Akt to follow. Trends in Biochemical Sciences 26, 657-664 (2001).; Platz, et al., Human cutaneous melanoma; a review of NRAS and BRAF mutation frequencies in relation to histogenetic subclass and body site. Molecular Oncology 1, 395-405 (2008).] To identify lncRNA transcripts that respond to MAPK pathway upregulation we transduced an NRAS^{Q61} mutant plasmid into primary human melanocytic cell lines (PHM^{Q61}). (FIG. 7a-b) PHM^{Q61} cells showed upregulated levels of phosphorylated ERK and AKT (pERK and pAKT). (FIG. 7c) Activating NRAS mutations like NRAS^{Q61} are commonly diagnosed in benign nevi and additional transformations are needed to fully unfold the malignant potential of melanocytes. [Poynter, et al. BRAF and NRAS mutations in melanoma and melanocytic nevi. Melanoma Research 16, 267-273 (2006)] No significant differences in cell proliferation could be measured comparing the PHM^{Q61} and PHM cell lines transduced with an empty vector (PHM^{e}), indicating that a sole NRAS^{Q61} mutation is not sufficient to equip melanocytic cell lines with profound melanoma cell characteristics. (FIG. 7b-d)

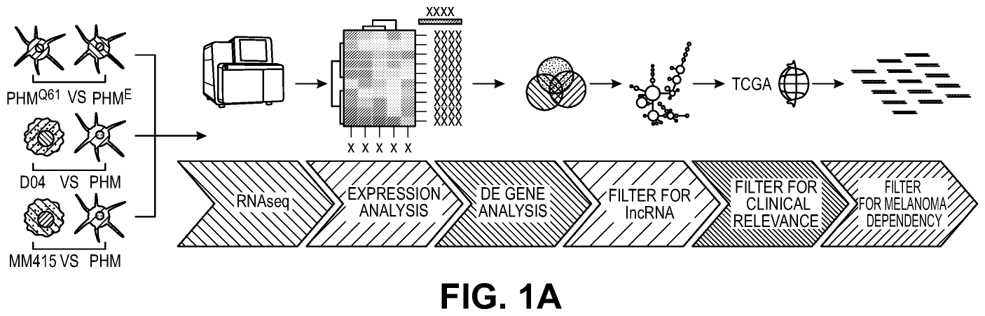

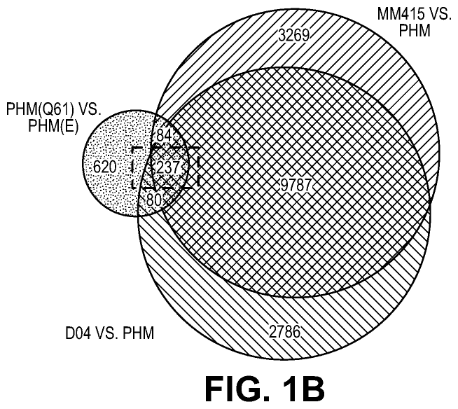

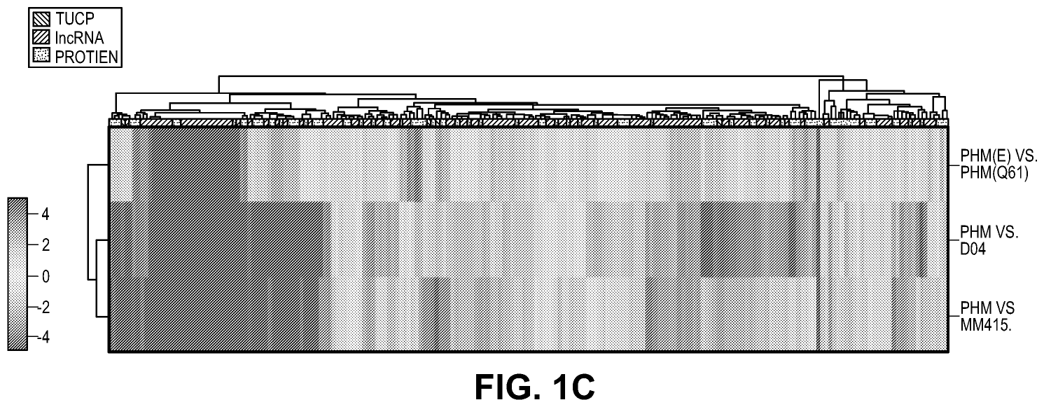

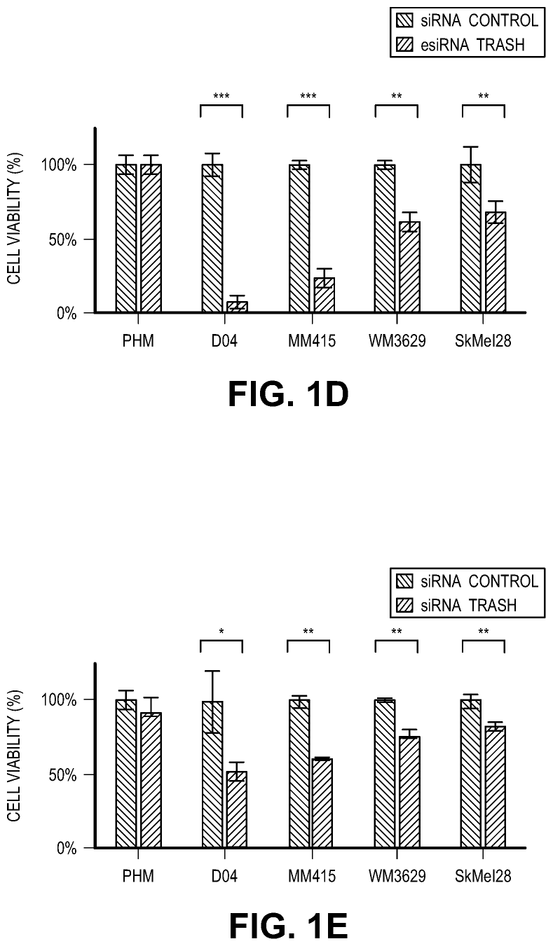

FIG. 1a represents a schematic workflow overview of the combined in silico and in vitro processes to identify MAPK pathway activation responsive lncRNAs that are essential for melanoma cell survival. First, we compared pair-end non-poly A enriched 101-bp RNASeq data from PHM, PHM^{E}, PHM^{Q61}, and two melanoma cell lines (D04, MM415) harboring MAPK pathway hyperactivating mutations. 237 transcripts were differently expressed (DE) in PHM^{Q61}, D04, and MM415 when compared to standard melanocytes (PHM^{Q61}ΔPHM^{E}; DO4ΔPHM; M415ΔPHM). (FIG. 1b-c) 120 of the DE genes were lncRNA transcripts. 28 of those transcripts were also expressed (FPKM values >0.2) in >90% of patient derived melanoma samples from the TCGA dataset This process led to the identification of several lncRNA transcripts that respond to MAPK pathway activation, including the transcript AC004540.4, which is located on the reverse strand of chromosome 7. Based on our functional studies, which will be discussed in later parts of this study, we named the novel transcript: TRanscript ASociated with HNRNPA2B1 (TRASH).

Endoribonuclease-prepared siRNA (esiRNA) is an efficient and specific method for RNAi screens in mammalian cells. [Kittler, R. et al. An endoribonuclease-prepared siRNA screen in human cells identifies genes essential for cell division. Nature 432, 1036-1040 (2004)] RNAi screening using TRASH targeting esiRNA libraries led to strong cell viability decrease in melanoma cell lines, while no such impact could be observed in melanocytic cell lines. (FIG. 1d) To reduce the chance of measuring off target effects, we subsequently conducted siRNA mediated RNAi screening. As expected, siRNA mediated TRASH silencing showed significant cell viability decrease in melanoma cell lines, but not melanocytic cell lines (FIG. 1e). These findings unveil that our pipeline identified a MAPK activation responsive lncRNA that is essential for melanoma cell survival.

TRASH is a Nuclear Regulator of hnRNPA2/B1

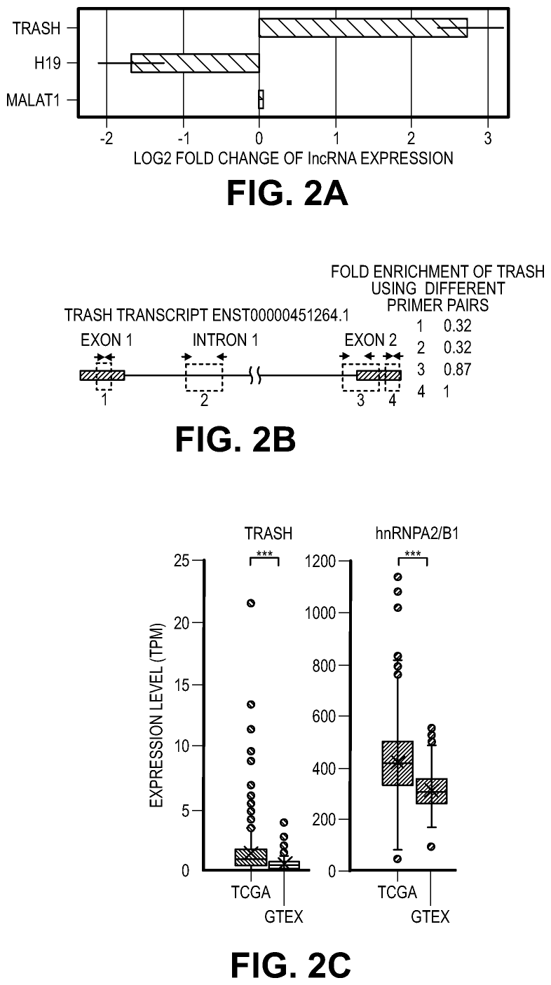

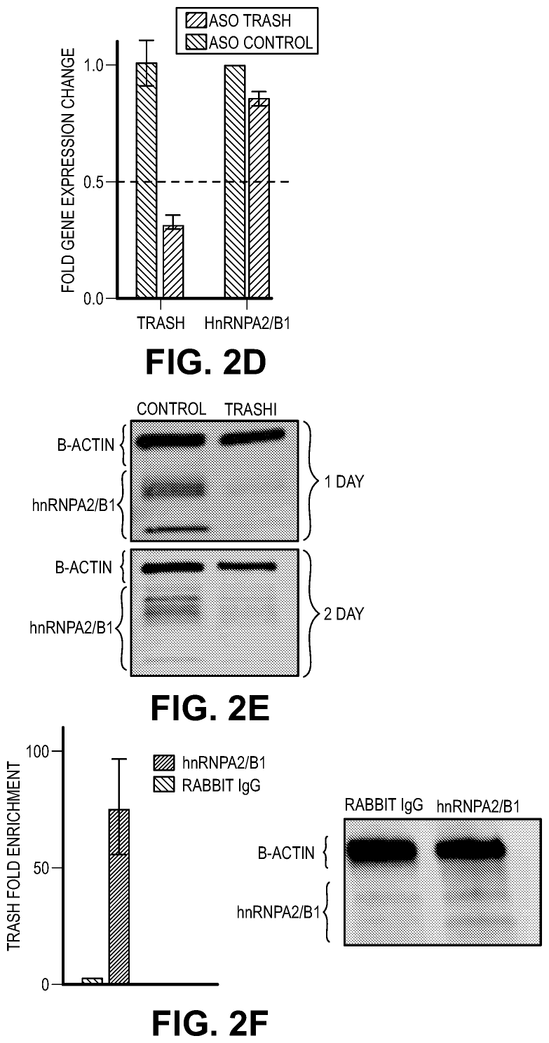

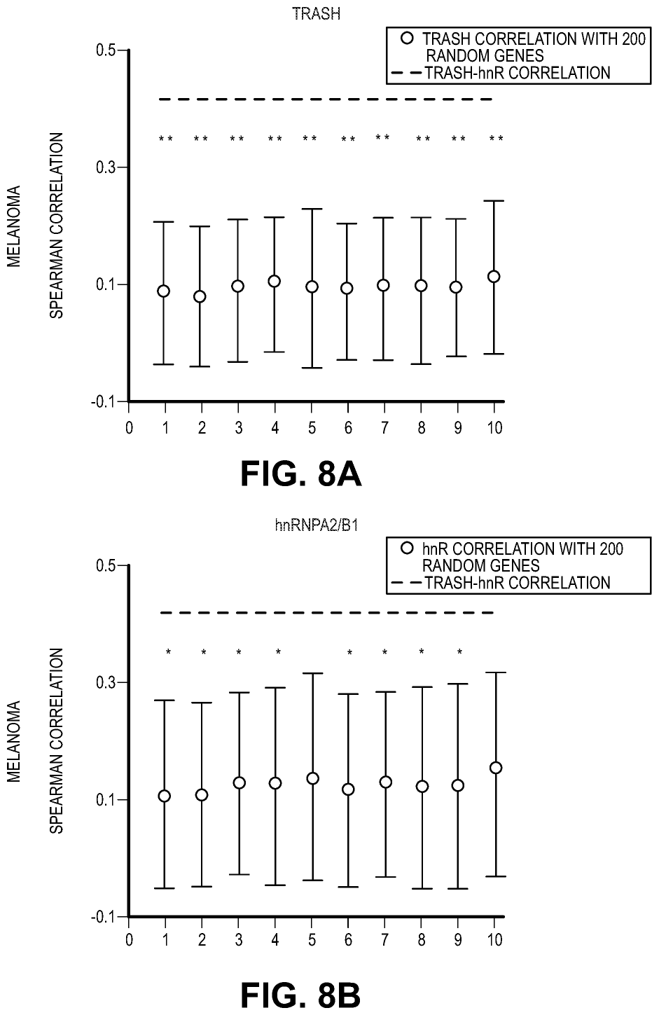

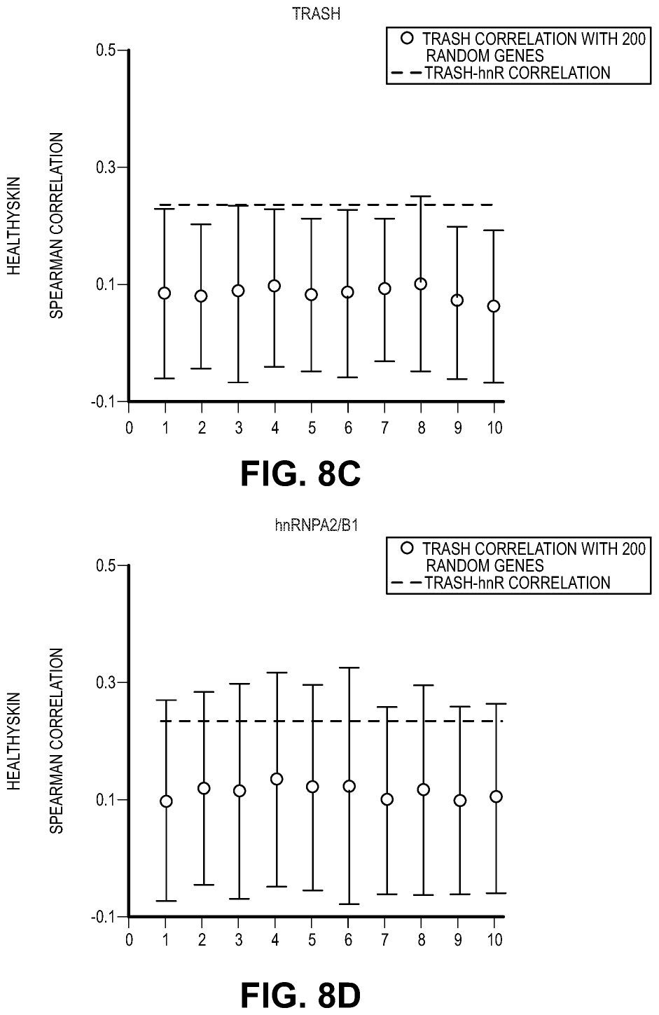

The regulatory functions of lncRNAs are closely related to their subcellular localization and lncRNAs are primarily localized to the nucleus. [Karakas, et al., The Role of LncRNAs in Translation. Noncoding RNA 7, 16 (2021).; Derrien, et al. The GENCODE v7 catalog of human long noncoding RNAs: Analysis of their gene structure, evolution, and expression. Genome Research 22, 1775-1789 (2012).] To identify the role of TRASH in melanoma, we performed subcellular fractionation followed by qPCR, demonstrating that TRASH is highly enriched in the nuclear compartment versus the cytoplasmic compartment in melanoma. (FIG. 2a) Nuclear enriched lncRNAs often exist in inefficiently spliced states. [Statello, et al. Gene regulation by long non-coding RNAs and its biological functions. Nat Rev Mol Cell Biol 22, 96-118 (2021)] Using 4 different primer pairs for comparison of relative quantification of different intronic/exonic regions of TRASH through qPCR further showed that exonic, intronic and exon/intron transition regions of TRASH were detected in different quantities, indicating that TRASH transcripts may exist to a certain extent in inefficient spliced states. (FIG. 2b) Genomic juxtapositioning of lncRNAs and protein coding genes can result in co-expression. The closest genomic same strand protein coding gene to TRASH is the oncogene coding for hnRNPA2/B1. [Statello, et al. Gene regulation by long non-coding RNAs and its biologicalfunctions. Nat Rev Mol Cell Biol 22, 96-118 (2021)] HnRNPA2/b1 is part of the family of heterogeneous nuclear ribonucleoproteins (hnRNPs), a group of protein[s that have at least one RNA-binding motif and regulate nucleic acid metabolism. [Singh, R. & Valcircel, J. Building specificity with nonspecific RNA-binding proteins. Nat Struct Mol Biol 12, 645-653 (2005).] HnRNPA2/B1 interacts with lncRNAs and exerts regulatory functions in MAPK pathway signaling. [Gupta, A. et al. The HNRNPA2B1-MST1R-Akt axis contributes to epithelial-to-mesenchymal transition in head and neck cancer. Lab Invest (2020) doi:10.1038/s41374-020-0466-8.; Barceló, C. et al., Ribonucleoprotein HNRNPA2B1 Interacts With and Regulates Oncogenic KRAS in Pancreatic Ductal Adenocarcinoma Cells. Gastroenterology 147, 882-892.e8 (2014).; Chen, Z. et al. Integrative Analysis of NSCLC Identifies LINC01234 as an Oncogenic lncRNA that Interacts with HNRNPA2B1 and Regulates miR-106b Biogenesis. Molecular Therapy 28, 1479-1493 (2020).; Liu, B. et al. Enzalutamide-Induced Upregulation of PCAT6 Promotes Prostate Cancer Neuroendocrine Differentiation by Regulating miR-326/HNRNPA2B1 Axis. Front. Oncol. 11, 650054 (2021).; Shen, Y. et al. lncRNA ST3GAL6-AS1 promotes invasion by inhibiting hnRNPA2B1-mediated ST3GAL6 expression in multiple myeloma. Int J Oncol 58, 5 (2021).; Wang, H. et al. Long noncoding RNA miR503HG, a prognostic indicator, inhibits tumor metastasis by regulating the HNRNPA2B1 NF-κB pathway in hepatocellular carcinoma. Theranostics 8, 2814-2829 (2018).; Shilo, A. et al. Splicing factor hnRNP A2 activates the Ras-MAPK-ERK pathway by controlling A-Raf splicing in hepatocellular carcinoma development. RNA 20, 505-515 (2014).] To identify possible co-interactions and dependencies of TRASH and hnRNPA2/B1 we explored the correlation between the genes of interest in contrast to permutations of randomly chosen genes in patient derived melanoma and healthy skin samples. Most notably, RNA expression of each gene is significantly higher in melanoma. (FIG. 2c) Correlation of TRASH and hnRNPA2/B1 is almost always significantly stronger in melanoma than the average correlation of each gene to 10 sets of random genes (p<0.05 10/10 for TRASH and 8/10 for hnRNPA2/B1). However, in healthy skin samples, no significant difference could be seen in any of the 20 comparisons. (FIG. 8a-d) Inhibition of TRASH expression did not significantly affect hnRNPA2/B1 RNA abundance, indicating that TRASH does not regulate hnRNPA2/B1 gene expression. (FIG. 2d) To investigate if inhibition of TRASH expression affects hnRNPA2/B1 protein expression, we visualized protein levels of hnRNPA2/B1 1 and 2 days after TRASH expression was inhibited. Immunoblot probing for HnRNPA2/B1 detected strong and stable protein expression reduction. (FIG. 2e) To investigate if the regulating effect of TRASH expression on hnRNPA2/B1 protein levels may rely on direct RNA-protein binding, we pulled down hnRNPA2/B1 from melanoma cell lysate and compared TRASH enrichment to negative control pulldown. HnRNPA2/B1 pulldown samples showed >65-fold enrichment of TRASH compared to the control samples, indicating that the lncRNA TRASH and the protein hnRNPA2/B1 directly interact. (FIG. 2f)

Taken together, these findings indicate that melanoma is characterized by TRASH and hnRNPA2/B1 upregulation and both molecules seem to physically interact with each other. Most notably, TRASH expression seems to be essential for maintaining stable hnRNPA2/B1 protein levels in melanoma.

### TRASH Serves as MAPK and PI3K-Akt Signaling Cascade Relevant Anti-Apoptotic Regulator in Melanoma.

It is common practice to use synthetic nucleic acids such as siRNA and Antisense Oligonucleotides (ASOs) for silencing gene expression and these methods have the potential to be widely used in future clinical therapeutic approaches. [Winkle, et al., Noncoding RNA therapeutics—challenges and potential solutions. Nat Rev Drug Discov 20, 629-651 (2021).; Deleavey et al. Designing Chemically Modified Oligonucleotides for Targeted Gene Silencing. Chemistry & Biology 19, 937-954 (2012)] Both methods can lead to off-target effects and unwanted immune system activation. [Kanasty, et al., Action and Reaction: The Biological Response to siRNA and Its Delivery Vehicles. Molecular Therapy 20, 513-524 (2012)] In contrast to siRNA, ASOs allow more chemical modification of synthetic nucleic acids to reduce unwanted side effects. [Kole, et al., RNA therapeutics: beyond RNA interference and antisense oligonucleotides. Nat Rev Drug Discov 11, 125-140 (2012).] Therefore, we focused on GapmeR-type ASO mediated TRASH inhibition (TRASHi) studies.

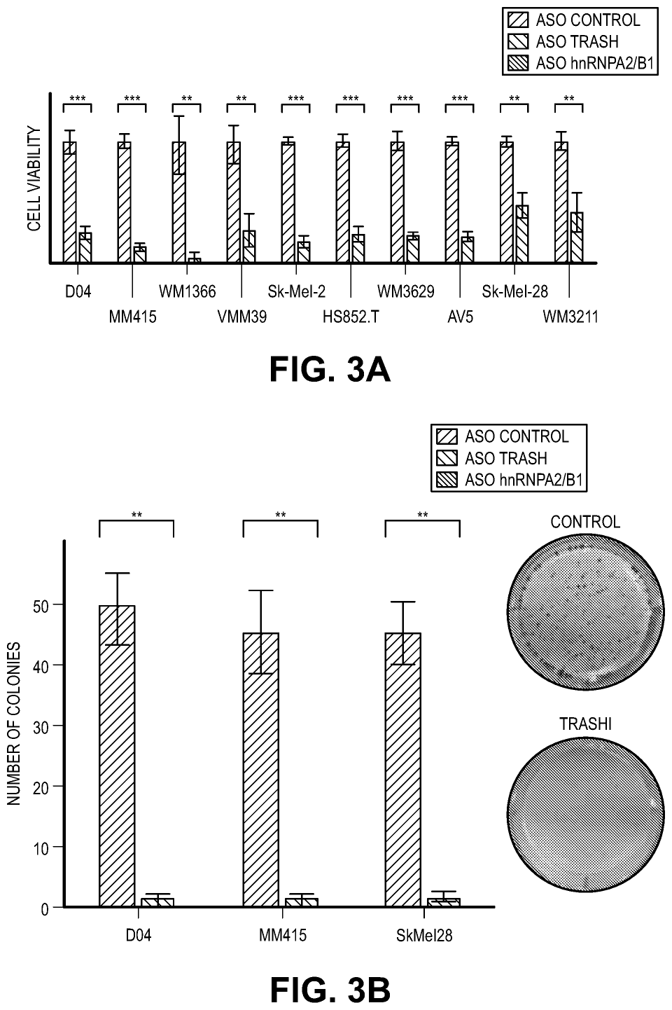

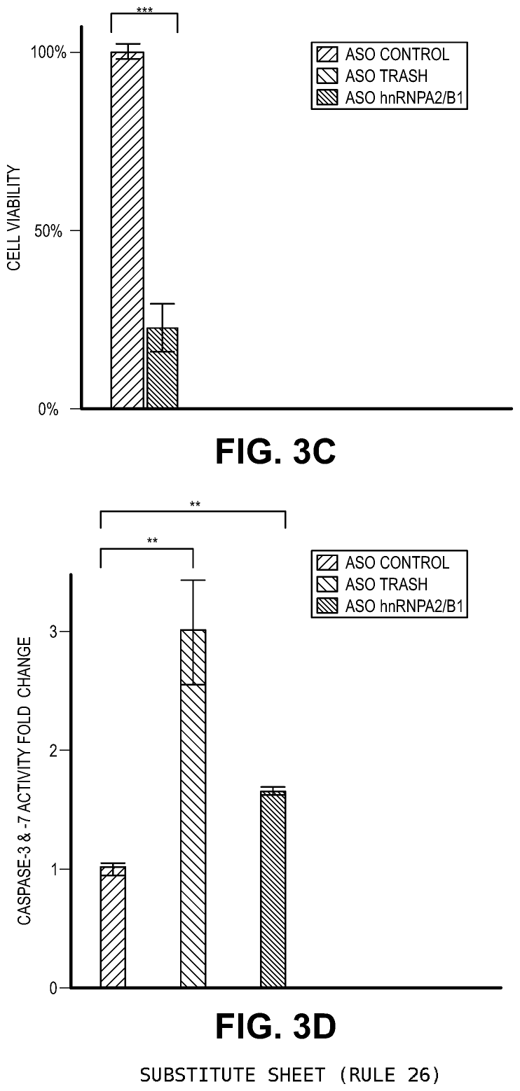

In the next step we tested TRASHi in a repository of standard and primary patient derived melanoma cell lines harboring MAPK pathway activating NRAS, BRAF and c-KIT mutations, which is frequently seen in melanoma patients. [Liang, J. et al. The C-Kit Receptor-Mediated Signal Transduction and Tumor-Related Diseases. Int. J. Biol. Sci. 9, 435-443 (2013); Vu, et al., Targeting mutant NRAS signaling pathways in melanoma. Pharmacological Research 107, 111-116 (2016); Dhomen, et al., BRAF Signaling and Targeted Therapies in Melanoma. Hematology/Oncology Clinics of North America 23, 529-545 (2009).] TRASHi induced a strong cell viability decrease in melanoma, but not in melanocytic cell lines. (FIG. 3a) To measure the impact of TRASHi on the reproductive viability of melanoma cells, we performed clonogenic assays on three different melanoma cell lines. TRASHi drastically reduced the capability of melanoma cells to produce colonies. (FIG. 3b) Also, ASO mediated inhibition of hnRNPA2/B1 expression (hnRNPA2/B1i) led to significant cell viability decrease. (FIG. 3c) Caspase 3 & 7 activity increase is a marker for apoptosis induction. [Luthi, et al., The CASBAH: a searchable database of caspase substrates. Cell Death Differ 14, 641-650 (2007).] Caspase-3 &-7 activity was significantly increased by 3-fold after TRASHi and 1.7-fold after hnRNPA2/B1 inhibition. (FIG. 3d)

To examine the functional relevance of TRASH in melanoma D04 cells were treated with TRASHi and non-targeting control ASOs, RNA was extracted and used for RNA-Seq. Differential expression (DE) analysis showed TRASHi had a global effect on melanoma gene expression. We found that 574 genes were down-regulated, and 493 genes were up-regulated. GO term analysis revealed the top enriched GO term cluster among the down-regulated genes is relevant to “ECM-receptor interaction” and “PI3K-Akt signaling pathway”; the top enriched GO term cluster among the up-regulated genes included terms like “protein tyrosine kinase activity (GO: 0004713)” and “Ras guanyl-nucleotide exchange factor activity (G00005088”). These GO terms consisted of genes encoding growth factors, tyrosine kinases, G protein coupled receptor subunits and collagen subunits.

These findings indicate that the functional mechanisms of TRASH are linked to genes situated at the top of the MAPK and PI3K-Akt signaling cascade. TRASH expression seems to be a common apoptosis inhibiting dependency in MAPK-pathway activated melanoma. Some of the anti-apoptotic functions of TRASH may rely to its stabilizing effect on hnRNPA2/B1. Furthermore, these findings allow the conclusion that TRASH may excise its regulatory functions upstream of many kinase-pathway cascades.

### Kinase Activity Profiling Reveals Unique Anti-Apoptotic Features of TRASH Expression

Considering the results that TRASH seems to serve as an anti-apoptotic regulator in melanoma that broadly affects kinase activity states, we used the novel technique of HTKAM to thoroughly investigate kinase activity shifts followed by TRASHi.

TRASH Knockdown Shows Characteristics that can be of High Clinical Value

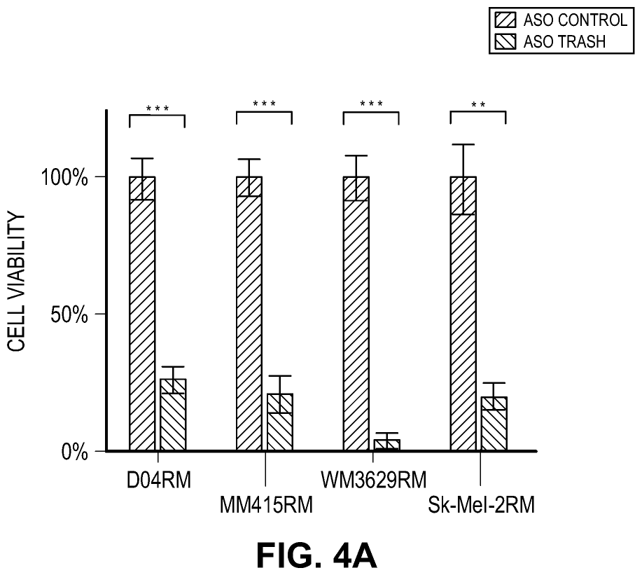

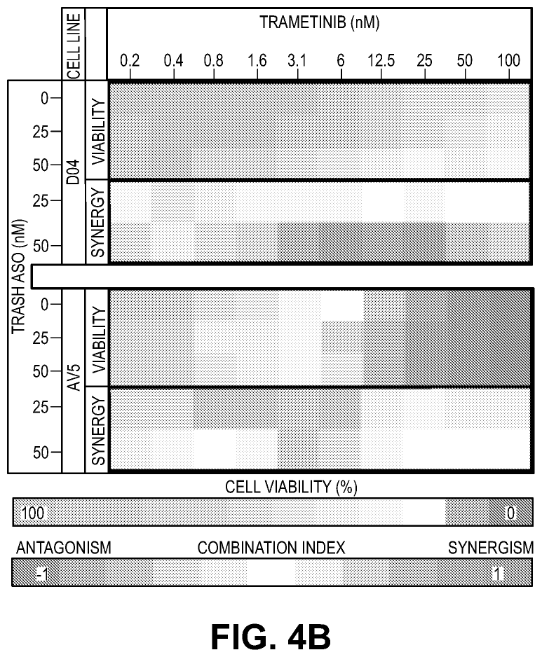

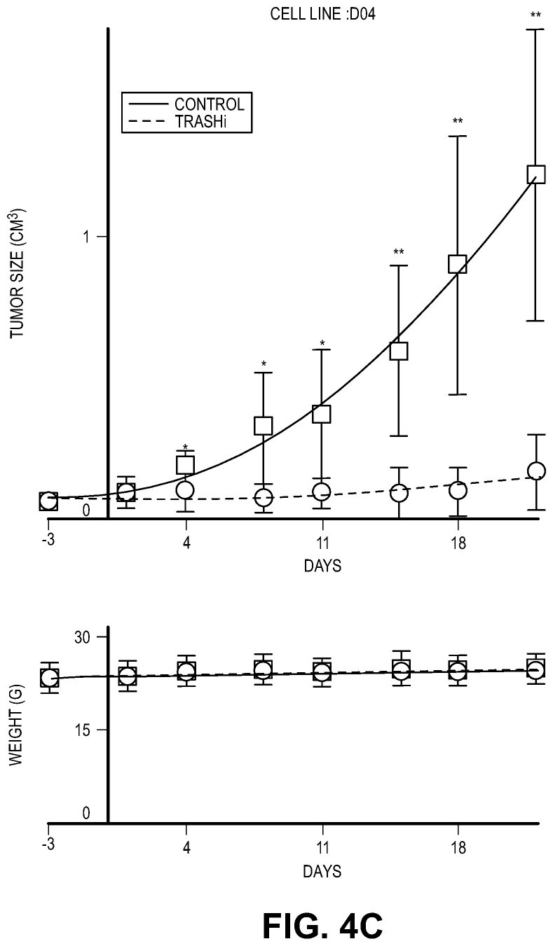

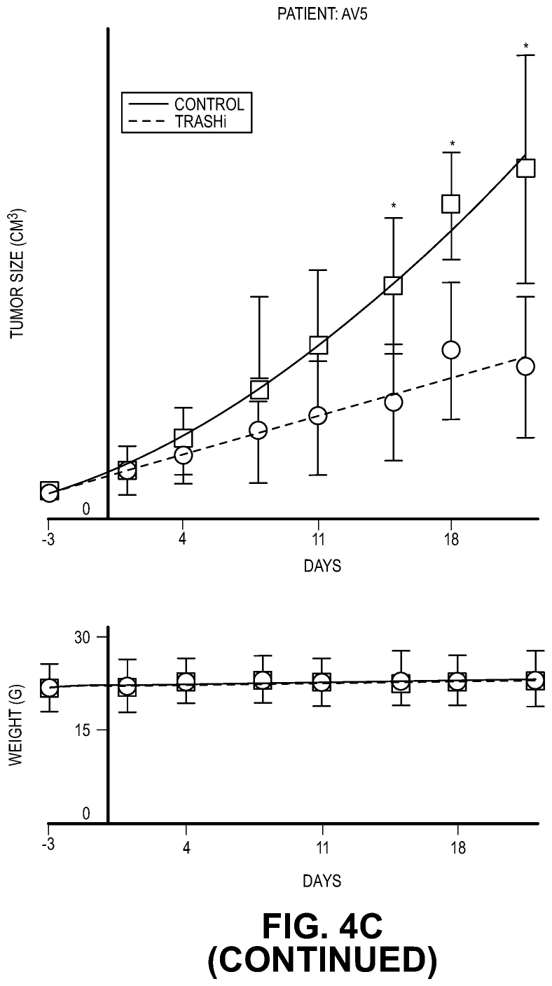

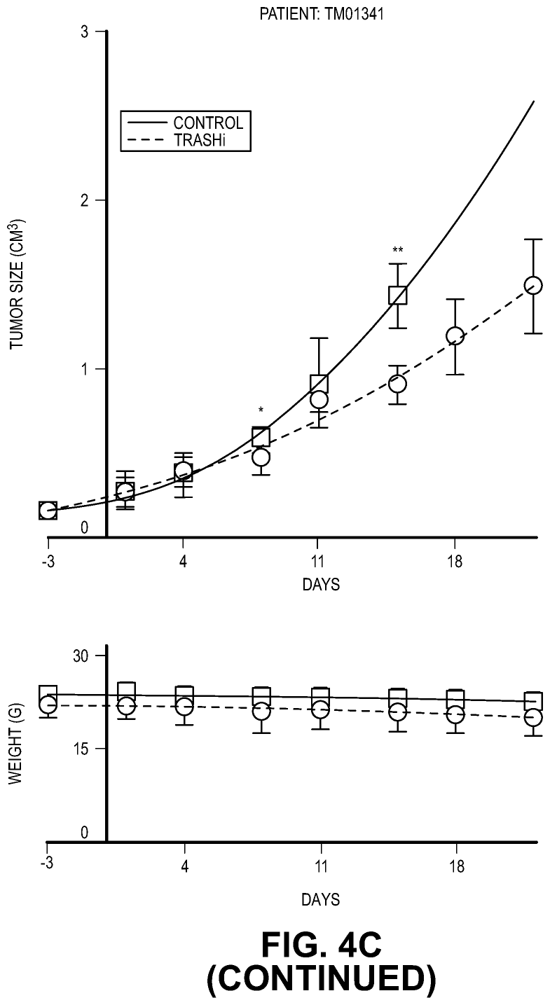

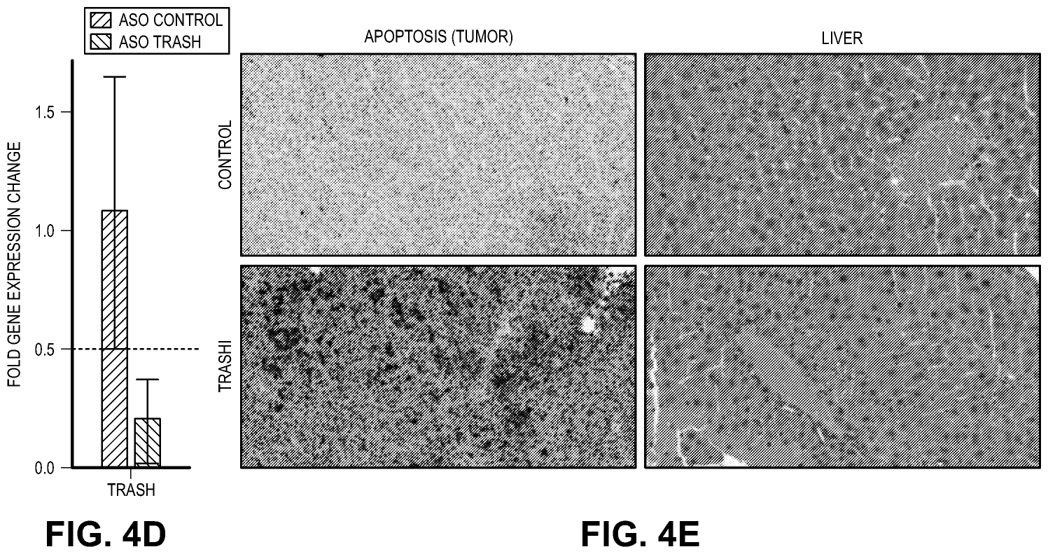

The MEK inhibitor (MEKi) trametinib is a FDA approved drug for the treatment of melanoma as mono- and combinatorial therapy and used in clinics worldwide. [Wright, et al., Trametinib: First Global Approval. Drugs 73, 1245-1254 (2013).] Drug resistance is the main limiting factor in modern oncology. [Vasan, et al., A view on drug resistance in cancer. Nature 575, 299-309 (2019).] Therefore therapeutic applications that reduce growth of drug resistant tumors are urgently needed. TRASHi in a panel of cell lines that are resistant to the MEK-Inhibitor Trametinib (MEKi) led to significant cell viability decrease, comparable to the effect seen in their nonresistant naive cell line counterparts. (FIG. 3a+4a) Combinational application of drugs is a common strategy in clinical oncology to synergize drug effects and to hamper the development of drug resistance. [Sawyers, C. L. Perspective: Combined forces. Nature 498, S7-S7 (2013); Kling, J. Bundling next-generation cancer therapies for synergy. Nat Biotechnol 24, 871-872 (2006).] Synergistic effects could be measured in a broad panel of concentration combinations in a standard melanoma cell line and in directly patient derived melanoma cells when testing dual TRASHi and MEKi. Synergy strongly increased with higher concentrations of TRASHi. More importantly, no notable inhibitory effects of could be observed. (FIG. 4b)

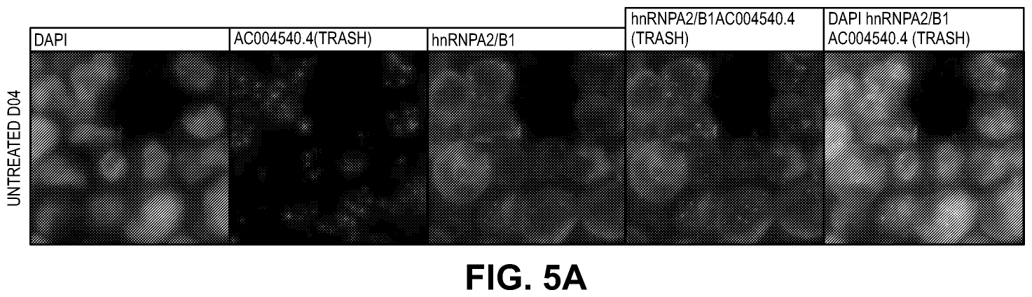

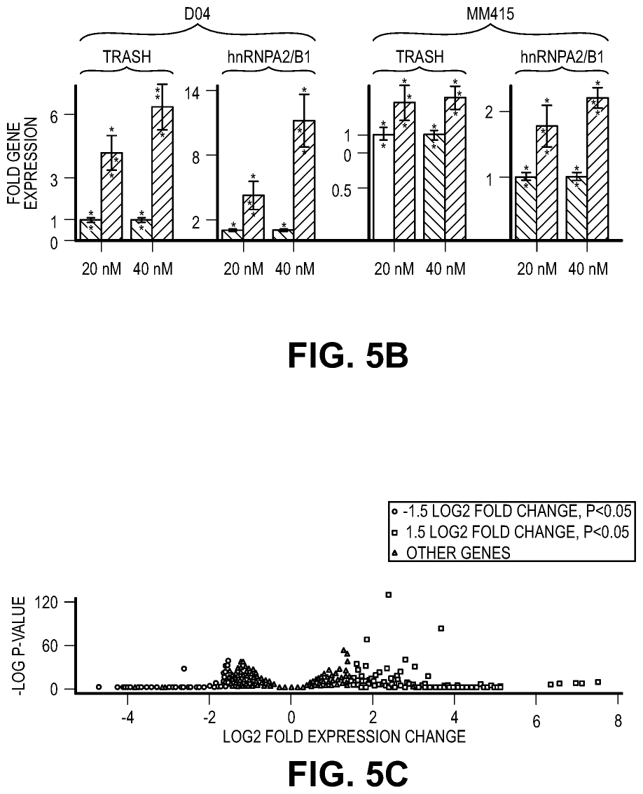

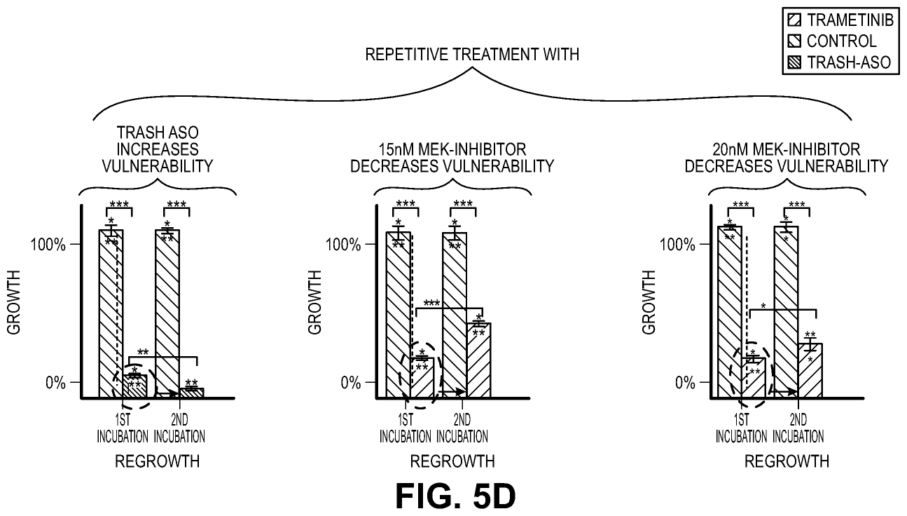

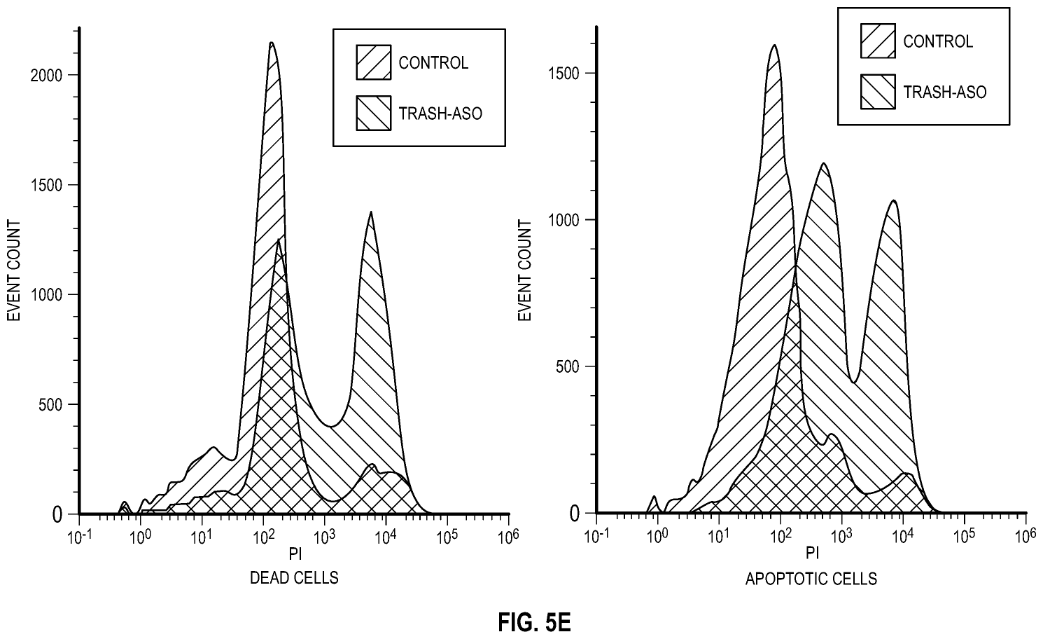

Next, we rescued cells that survived initial TRASH knockdown and after a phase of regrowth in ASO free media, we repeated TRASHi. Cells that survived initial TRASHi kept their vulnerability to TRASHi. (FIG. 5d) To further evaluate the clinical potential of targeting TRASH dependency in melanoma, we aimed to test the effects of TRASHi in mouse models. ASO mediated inhibition of RNA expression has been proven to lead to effective tumor growth reduction in vivo. [Shi, L. et al. A KRAS-responsive long non-coding RNA controls microRNA processing. Nat Commun 12, 2038 (2021).; Leucci, E. et al. Melanoma addiction to the long non-coding RRNA SAMMSON. Nature 531, 518-522 (2016).] We used xenograft models harboring a standard melanoma cell line (D04), patient derived primary melanoma cells (AV5) and a melanoma PDX model. A treatment regimen of 60 ug subcutaneous ASO injections twice a week, co applied with an in vivo transfection reagent, reduced tumor growth in all three mouse models significantly. (FIG. 4c) The PDX tumor model TM01341 showed extremely high rates of tumor growth. While tumor growth could be significantly hampered in the TRASHi group, mice of the control group had to undergo euthanization before desired endpoint of the experiment, due to UCSF-IACUC guidelines for maximum acceptable tumor sizes. To simulate the experiment to the desired endpoint, we tumor growth in the PDX control group was forecasted using a regression model. (FIG. 4c) In none of the three melanoma type groups significant differences in weight change could be seen in between the TRASHi and control treatment group. (FIG. 4c) Essentially, RT-qPCR of tumor tissue extracted after end of treatment period showed that in vivo TRASHi strongly reduced TRASH expression. (FIG. 4d) In some circumstances GapmeR ASOs can show toxic side effects, in particular hepatotoxicity. [Kasuya, T. et al., Ribonuclease H1-dependent hepatotoxicity caused by locked nucleic acid-modified gapmer antisense oligonucleotides. Sci Rep 6, 30377 (2016)] Liver tissue of treated mice was extracted for H+E staining after end of treatment period. Neither for animals receiving TRASHi, nor for animals receiving control ASOs detectable pathologic changes in liver tissue could be shown. (FIG. 4e) Furthermore, IHC staining of tumor tissue that was harvested from mice at the end of treatment period shows high levels of the apoptosis marker cleaved caspase 3 in tumors that underwent TRASHi compared to tumors of mice that received control ASO. (FIG. 4e)

In summary these findings show that TRASHi could help to bypass the recent limitation of MEKi resistance in clinical melanoma therapy and also has the potential amplify MEKi treatment. To our knowledge no data regarding resistance building against GapmeR ASO mediated RNA depleting therapy in mammalian cells exists yet. Our findings highlight that no early onset treatment resistance building could be observed for TRASHi in melanoma. Additionally, TRASHi significantly reduces TRASH expression and tumor growth in vivo while showing no signs of toxicity.

### Discussion

MAPK pathway activation is a common and initiating event in melanoma genesis and regulating elements of its protein kinase cascades serve as effective targets for oncological treatment. [Luke, et al., Targeted agents and immunotherapies: optimizing outcomes in melanoma. Nat Rev Clin Oncol 14, 463-482 (2017).; Hodis, E. et al., A landscape of driver mutations in melanoma. Cell 150, 251-263 (2012).] There has been major progress in the development of melanoma therapeutics in the past 10 years. However, many patients do not benefit from these advances due to initial or acquired treatment resistance. Therefore, additional treatment options are urgently needed. Here, we present a bioinformatic pipeline that is composed of analytical steps including a broad set of in silico and in vitro derived data which lead to the identification of the oncogenic lncRNA TRASH. TRASH expression is responsive to MAPK activation and essential for MAPK-dependent melanoma cell survival. Our findings highlight the potential of TRASH as a therapeutic RNA target in melanoma.

With the ultimate goal of being able to develop a method of TRASH silencing with clinical utility, we used ASOs to inhibit TRASH expression (TRASHi), a gene silencing method that has already been utilized in clinical trials for various diseases. [Bedikian, et al., Dacarbazine with or without oblimersen (a Bcl-2 antisense oligonucleotide) in chemotherapy-naive patients with advanced melanoma and low-normal serum lactate dehydrogenase: ‘The AGENDA trial’. Melanoma Research 24, 237-243 (2014).; Beer, T. M. et al. Custirsen (OGX-011) combined with cabazitaxel and prednisone versus cabazitaxel and prednisone alone inpatients with metastatic castration-resistant prostate cancer previously treated with docetaxel (AFFINITY): a randomised, open-label, international, phase 3 trial. The Lancet Oncology 18, 1532-1542 (2017).]

TRASHi induces apoptosis and inhibits colony formation capabilities in a broad panel of MAPK-dependent melanoma cell lines and primary melanoma cells, while having no effect on melanocytic cell lines. Our findings highlight that TRASH may exert regulatory mechanisms upstream of the MAPK and PI3K-Akt pathway. Some of the oncogenic features of TRASH may rely on the stabilizing effect it exerts on its protein binding partner which is the product of the anti-apoptotic oncogene hnRNPA2/B1. Analysis of patient derived melanoma and non-melanoma skin tissue points toward the direction that gene expression correlation and upregulation of TRASH and hnRNPA2/B1 expression may be a melanoma specific mechanism. Therefore, we propose that the functional axis of TRASH and hnRNPA2/B1 is concomitant of melanoma.

Our results underline the high clinical potential of TRASHi. The precise role of TRASH expression in drug resistance mechanisms to melanoma goes beyond the scope of this research project. However, we show that MEKi-resistance does not desensitize melanoma cells to their TRASH dependency, indicating that TRASHi can serve as treatment for melanoma refractory to small molecule MEK inhibiting therapy. Dual application of TRASHi and MEKi amplifies the effects of mono-application, demonstrating the synergistic effects of multi-drug regimens that clinical dermato oncologists strive for.

Consistent with our in vitro results, we show that TRASH silencing is a powerful tool to reduce tumor growth through apoptotic cell death in PDX and xenograft melanoma mouse models, while showing no signs of hepatotoxicity or TRASHi-related weight loss.

MAPK hyperactivation propels expression of essential oncogenic elements and our findings indicate that the lncRNA TRASH is one of them. We unveiled a network of anti-apoptotic kinases that are affected by TRASHi and to our knowledge, such a pattern of apoptosis specific kinase activity shifts through inhibiting a druggable lncRNA target have never been reported.

Given the robust anti-melanoma effects of TRASHi and the development of RNA targeting therapy as a promising new method in next generation cancer therapy, we propose that TRASHi is a promising lncRNA targeting cancer therapy, from which many patients, including the subset of melanoma patients with MEKi resistance, could benefit.

### Methods

Bioinformatic Pipeline for Identifying MAPK-Responsive lncRNAs

### Reference Annotation

A custom reference annotation of total 75,506 transcripts, referring to 35,101 genes, of which 16,405 were classified as noncoding, was built by integrating 13,870 lncRNA genes from the GENCODE (V19, July 2013 freeze, GRCh37, downloaded March 2015) into the RefSeq database (release 57, downloaded March 2013). Cuffcompare (version 2.1.1) was used to cut out redundant transcripts.

Assembly and Identification of Previously Unidentified lncRNAs

After alignment to the human genome with TopHat (version 2.0.11), the reads were assembled into transcripts with Cufflinks (version 2.1.1). To discover novel lncRNAs, we excluded all transcripts that overlapped with any genes from our initial reference annotation. To filter out transcriptional noise, we kept only multi-exonic transcripts which were >200 bp and had at least one intron region >10 bp. Next, isoforms were merged with Cuffcompare into 1,311 transcripts.

### Coding Potential Assessment of Transcripts

To identify transcripts with a coding potential, we ran (i) the HMMER3 algorithm (considering all 6 open reading frames) for each of the 1,311 transcripts to identify any protein family domain as noted in the Pfam database (release 27.0, Pfam-A and Pfam-B domains considered) and (ii) the Coding Potential Assessment Tool (CPAT v1.2.1). 479 transcripts were categorized as TUCPs (331 transcripts called by Pfam only, 70 transcripts called by CPAT only, and 78 transcripts called by both). The other 832 transcripts were classified as previously unidentified lncRNAs, or “novel lncRNAs”. The final reference annotation had a total of 76,817 transcripts referring to 35,961 genes.

### Filter for DE Genes

Cuffdiff (v.2.1.1) was used to identify differential gene expression analysis between PHM^{E} and PHM^{Q61}. From a reference of 35,905 genes, we discarded genes with FPKM <0.2 in both conditions (14,790 genes) and kept genes with log 2fold change >1 or <−1 (1021 genes). Cufflinks was used to obtain FPKM values of the 1021 genes in Seq-Data from the D04 and MM415 melanoma cell lines. Log 2 transformations were performed to calculate expression fold change in the comparisons: 1) PHM^{E} vS. PHMQ61, 2) PHM vs. D04, 3) PHM vs. MM415. The value of 1 was added to all FPKM values before calculating log 2fold change. Genes that had a log 2-fold change >1 or <−1 were considered as differentially expressed.

### Animal Models

Rodent experimental procedures were approved by the Office of Research institutional Animal Care and Use Program (IACUC) at the University of San Francisco (UCSF). All in vivo studies were conducted under the authorized protocol number AN174613-03. Mice were maintained in a pathogen free environment and had free access to food and water. For PDX tumor models, the PDX type TM01341, derived from liver metastasis of a male melanoma patient was engrafted on 4- to 6-week-old NOD.Cg-Prkd^{scid} Il2rg^{tmlwjl}/SzJ mice (Stock.no 005557) on the right posterior dorsal flank (n=4/group). For cell line models 2×10\hat{ }⁶ D04 (n=5/group) and AV5 (n=3/group) cells in 150 ul of PBS and 50 ul of Matrigel were subcutaneously injected on the right posterior dorsal flank of 4- to 6-week-old homozygous nude Foxn1^{nu}/Foxn1^{nu} mice (Stock.no 007850). All mice were purchased and PDX tissue was engrafted from the vendor Jackson laboratory. Tumor size was measured using a digital caliper and the formula 0.5×(length×(width\hat{ }²)) was used to calculate tumor volume. Mice were treated twice a week with 60 ug of TRASH targeting ASOs, or 60 ug of non-targeting control ASO and 9.6 ul of in vivo JetPEI diluted in an overall amount of 200 ul 5% glucose. ASO injections were applied subcutaneously in a 2 cm distance to the tumor for a total of 7 injections. Mice were weighted twice a week and constantly observed for signs of distress or disorder. Mice were euthanized after three weeks of ASO application or when tumors reached a diameter of >2 cm. All experiments were performed in accordance with the UCSF Laboratory Animal Resource Center (LARC) guidelines. After euthanasia parts of tumors and liver tissue were excised and fixed in formalin solution, followed by storing in 70% ethanol and Immunohistochemistry staining. Parts of tumors were stored in RNAlater™ Stabilization Solution (ThermoFisher) and stored at −20° C. TRIzol solution was used to extract RNA from tissue and qPCR was performed to analyze gene expression.

### Cell Culture

Human melanoma cell line VMM39 was purchased from American Type Culture Collection (ATCC). Human melanoma cell lines D04, MM415, WM1366, WM3629, WM3211, Sk-Mel-2 and Sk-Mel-28 were a generous gift from Boris Bastian at the University of California, San Francisco. Primary human melanoma cell line Hs852.T was purchased from the Cell Culture Core Facility (CCCF) at the University of California, San Francisco. Primary human melanoma cell line AV5 was obtained from metastasis of a melanoma patient. All experimental protocols were approved by UCSF Human Research Protection Program Institutional Review Board (IRB #12-0948), all patients signed informed consent, and methods were carried out in accordance with relevant guidelines and regulations. Resistant cell lines DO4RM, MM415RM, Sk-Mel-2RM and WM3629RM were established as previously described. [Sanlorenzo, M. et al., The lincRNA MIRAT binds to IQGAPI and modulates the MAPK pathway in NRAS mutant melanoma. Sci Rep 8, 10902 (2018).] Primary human melanocytic cell lines (PHM) from infant foreskin of five healthy donors were available in our cell repository and pooled. Melanoma cell lines were maintained in RPMI 1640 media supplemented with 10% (vol/vol) heat inactivated fetal bovine serum. Melanocytes were maintained in M254 medium with HMGS supplements (1× final solution). All cell lines were incubated at 37° C. under 5% CO2.

### Viral Transduction

NRAS^{Q61R} cDNA was cloned into the Gateway entry vector pENTR/D-topo. pENTR/D-topo-NRAS^{Q61R} was subjected to site-directed mutagenesis to generate mutants which were then validated by Sanger sequencing. NRAS^{Q61R} cDNA in pENTR was cloned into the Gateway cloning-enabled destination vector gFG12. After lentiviral transduction, cells were grown for 2 weeks followed by cell sorting facilitating GFP intensity on a FACS Aria II cell sorter.

### Cell Fractionation

Total nuclear and cytoplasmic extracts were obtained using the SurePrep Nuclear/Cytoplasmic RNA purification kit according to the manufacturer's instructions. Primers are listed in supplementary table 1.

### Sanger Sequencing

RNA from PHME and PHMQ61 was extracted using Purelink RNA extraction kit (ambion) and transcribed into cDNA. Sanger Sequencing was performed using standard protocol by Quintarabio. Primers are listed in supplementary table 1.

### Protein Extraction and Immunoblotting

Total protein lysates were homogenized in 1×RIPA buffer and Halt protease and phosphatase inhibitor cocktail (1× final concentration) followed by centrifugation at 14,000 RPM/minute at 4° C. Protein concentration was quantified using the Pierce BCA Assay Kit (ThermoFisher Scientific). Linear absorbance was measured using the BioTek SynergyHT plate reader. Total protein in 1× Laemmli buffer with 10% 2-mercaptoethanol was separated by SDS/PAGE, transferred for 15 h to a PVDF membrane (IPVH00010; Millipore) by electroblotting with 20% (vol/vol) methanol, and blocked for 1 h in in Intercept (TBS) blocking buffer (LICOR). Membranes were incubated overnight at 4° C. with primary antiserum for hnRNPA2/B1 (abcam, cat.no.: ab31645, dilution 1:750) and Beta-Actin (Cell signaling, cat.no.: 8457, dilution 1:2500) following incubation with secondary Goat Anti-Rabbit serum (LI-COR, cat.no.: 925-68071, dilution 1:5000) for 1 h and scanned using the Li-COR Odyssey Imaging system.

RNA Extraction and Quantitative Real-Time PCR (qRT-PCR)

TRIzol, Phenol:chloroform:isoamyl alcohol (125:24:1) or NucleoSpin RNA kit (TaKaRa) was used for extracting Total RNA from cells and tissues according to the manufacturer's instructions. Total RNA was quantified by NanoDrop ND-1000 (Thermo Scientific) or Quibit 4 (Thermo Fisher). 50 ng or RNA was reverse transcribed using the cDNA synthesis and gDNA removal QuantiTect Reverse Transcription Kit. Real time PCR was performed using the iTaq Universal SYBR Green Supermix, 10 ng (20 ng for RIP Assay) of cDNA and on a QuantStudio™ 5 Real-Time PCR System or a 7500 fast real time PCR system. Relative gene expression was calculated using the comparative Ct method, normalized to GAPDH or β-actin. Primer sequences are listed in Supplementary Table 1.

### Oligonucleotide Transfection

EsiRNA was generated following standard protocol. [Kittler, R. et al., Genome-wide resources of endoribonuclease-prepared short interfering RNAs for specific loss-of-function studies. Nat Methods 4, 337-344 (2007).] Primer sequences TCACTATAGGGAGAGACACTCAAAGCCTGAGTAACAGA and TCACTATAGGGAGACTGACTGAGATTTTATTGAGCTGTG were used to create TRASH targeting esiRNA. SiRNA was purchased from Dharmacon, using the siDESIGN software. For TRASH targeting siRNA design, the sequence ACAAAGAGAGACAGGAAAUUU was used. For pooled non-targeting control siRNA design, the sequences UGGUUUACAUGUCGACUAA, UGGUUUACAUGUUGUGUGA, UGGUUUACAUGUUIUUCUGA and UGGUUUACAUGUUIUUCCUA were used.

ASO GapmeRs were purchased from QIAGEN and designed using the GeneGlobe design and analysis hub. For TRASH targeting ASO design, the sequence GACTGGAGATAATTAA was used for in vitro experiments and TGCGCGGCGGAAAGAA for in vivo. For hnRNPA2/B1 targeting ASO design, the sequence GACCGTAGTTAGAGG was used. For non-targeting control ASO design, the QIAGEN standard sequence AACACGTCTATACGC was used.

EsiRNA, siRNA and ASO GapmeRs were transfected in a final concentration of 50 nM unless mentioned otherwise and the transfection reagent Lipofectamine 3000 (2 ul/ml) was added according to the manufacturer's instructions.

### Expression Analysis in TCGA and GTEx

The analysis of TCGA/GTEx gene expression data was done in R. For TCGA data, the SKCM dataset (n=469) was used. The GDCquery function of the TCGAbiolinks package was run with the following parameters: project=“TCGA-SKCM”, data.category=“Transcriptome Profiling”, data.type=“Gene Expression Quantification”, workflow.type=“HTSeq−FPKM”. GDCdownload and GDCprepare then produce a RangedSummarizedExperiment. Expression values are then stored in a data frame and converted to TPM by dividing each FPKM value by the total FPKM of each sample and multiplying by 10\hat{ }⁶. To retrieve GTEx data (n=394), “GTEx_Analysis_2017-06-05_v8_RNASeQCv1.1.9_gene_tpm.gct.gz” was downloaded from gtexportal.org/home/datasets. Skin samples within the GTEx dataset were identified by referencing https://www.ebi.ac.uk/arrayexpress/experiments/E-MTAB-5214/samples/?s_page=59&s_pagesize=500&s_sortby=col_8&s_sortorder-ascending. The raw read counts were converted to TPM values and then transformed to log 2 scale. A value of 1 was added to avoid taking log of zero. For both TCGA and GTEx, duplicate genes were removed. If a patient provided multiple specimens, only the first would be used. The ensemble ID for our genes of interest were ENSG00000225792 (TRASH) and ENSG00000122566 (hnRNP2/B1). Cor.test was applied to find the correlation between each gene and TRASH, and the same for hnRNPA2/B1. Spearman's correlation coefficient (ρ) was used to measure rank correlation. 2000 random genes were sampled from both datasets. The correlation of TRASH and hnRNPA2/B1 was ranked against 200 random gene correlations with TRASH and hnRNPA2/B1 each for 10 iterations.

### Cell Viability Assay

Dependent on cell doubling time, 0.7-2×10\hat{ }³ cells were seeded in 96 well plates. 1 day after seeding cells were incubated in media with oligonucleotide concentration and/or MEKi and transfection reagent. 3 (synergy experiments) or 5 (solely ASO) days after transfection Total luminescence was measured on the SynergyHT plate reader (BioTek) using Gen5 software. Cell viability decrease always is shown in relation to cell viability of cells incubated with non-targeting control ASOs.

### Caspase Glo 3/7 Assay

Dependent on cell doubling time, 2-3×10\hat{ }³ cells were seeded in 96 well plates. 1 day after seeding cells were incubated in media with 50 nM oligonucleotide concentration and transfection reagent. 1 day after transfection Total luminescence was measured on the SynergyHT plate reader (BioTek) using Gen5 software. Experiments were performed in quadruplicates.

### RNA-Binding Protein Immunoprecipitation

The Magna RIP™ Kit (Millipore) was used following standard protocol. 10ug of Antibody for Rabbit IgG (Millipore, Cat.no.: PP64B) and hnRNPA2/B1 (Proteintech, Cat.no.: 14813-1-AP) was used to load magnetic beads. RNA precipitate was subjected to qRT-qPCR analysis.

### Colony Formation Assay

Dependent on cell doubling time, 1-2×10\hat{ }³ cells were seeded in 6 well plates. 1 day after seeding cells were incubated in media with 50 nM oligonucleotide concentration and transfection reagent. 6 days after transfection, cells were washed with PBS, fixed with 10% neutral buffered formalin, and stained with 0.1% crystal violet solution. Colonies were defined as cell conglomerates with >50 cells. Digital Images of plates were evaluated by two independent reviewers for colony counts. The final counts were calculated as the average count of both reviewers for all triplicates.

### Statistics and Reproducibility

Error bars in all the plots indicate mean±S.D. P-value <0.05 was considered statistically significant. ***p-value <0.001, **p-value <0.01, *p-value <0.05 by one tailed Student's t-test. All experiments were performed at least three times, unless otherwise indicated. Statistics was calculated with Microsoft Excel Version 2107.

### RNA Sequencing

Total RNA was isolated using the RNeasy mini Kit (QIAGEN) following the manufacturer's protocol. Quality check for extracted RNA was done using 2100 Bioanalyzer (Agilent Technologies, USA) or Tapestation System (Agilent Technologies, USA). All samples had a RIN score >8. cDNA sequencing libraries were prepared using the Illumina TruSeq Total RNA Sample kit. For samples used for identification of MAPK-responsive lncRNAs, paired-end, 101-bp sequencing was performed by Centrillion Genomic Services (Centrillion Biosciences, USA) on an Illumina HiSeq 2000. For DE gene analysis of ASO-transfected D04 samples, paired-end, 2×150-bp sequencing was performed by Genewiz (USA) on a Illumina HiSeq.

Sequence reads were aligned to the human genome (hg19) using TopHat (Version 2.0.11).

### Analysis of TRASHi Induced DE Gene Expression

Differential expression (DE) analysis was done using DESeq2. Differentially expressed genes were defined by more than 1.5-fold changes (log 2 >0.58 or <−0.58) in expression with FDR<0.05. GO term analysis was done using DAVID Functional Annotation Clustering analysis.

SUPPLEMENTARY TABLE 1
List of all primers and the according experiments
that they were used for

| Target | Experiment | Forward | Reverse |
|---|---|---|---|
| NRAS | Sanger Sequencing | CGCACTGACAATCCAG | TCGCCTGTCCTCATGTATT |
| | | CTAA | G |
| TRASH | Subcellular enrichment | TCACAACACACTCAAA | ACCCAACTGCACTCCAAAT |
| | | GCCTG | G |
| TRASH 1 | knockdown evaluation, | TCACAACACACTCAAA | ACCCAACTGCACTCCAAAT |
| | splicing efficiency, | GCCTG | G |
| | RIP | | |
| TRASH 2 | splicing efficiency | TAGCAGCAAAGACAA | TTAGCTGCGCAAACTCTGG |
| | | GCGGT | T |
| TRASH 3 | splicing efficiency | CATCATGACAGTGAGC | TTCCCCCTCTCTTCTTTTC |
| | | TTTAGGT | CAG |
| TRASH 4 | splicing efficiency | CATCGGCGTTTAAGGC | CGCTACGGTGACGATTCTG |
| | | AGC | G |
| hnRNPA2/ | knockdown evaluation | ATGGGAGAGTAGTTG | TCAGTATCTTCTTTAATTC |
| B1 | | AGCCAAA | CGCC |

### Example 2

We mapped and compared the phospho-catalytic profile of kinases of D04, MM415 and D04RM cells that were incubated with TRASH targeting ASOs (SEQ TD NO: 15), ASOs targeting the oncogenic lncRNA Malat1 and non-targeting control ASOs. Therefore, ye used the high-throughput system HTKAM to measure the enzymatic activity of kinases using biological peptide targets as phospho-sensors to reveal kinase dependencies in cell lines.

The results show significantly decreased activity levels of the kinases CDK1, LYN, YES1, (CHEK1, PKA, PKCa, PIM1 and the kinases of the Akt-family. These kinases fulfill an anti-apoptotic function in cells. The observed effect is specific to TRASH-inhibition and not a general effect that is seen upon ASO targeting of lncRNAs, as no such kinase activity shifts could be measured upon Malat1 inhibition.

### Example 3

ASO targeting BX470102.3 (SEQ ID NO: 13) leads to significant cell viability decrease in melanoma (D04, MM415, WM1366, VMM39, Sk-Mel-2, Hs852.T, Hs940.T, WM3629, AV5, AV4, Sk-Mel-28, WM3211, A375, MM485, WM3060, Sk-Mel-5), trametinib resistant melanoma (D04RM, MM415RM, Sk-Mel-2-RM, WM3629RM), Glioblastoma (U138-MG, T98G, A-172, U87-MG), Neuroblastoma (Sk-N-AS), multiple myeloma (H929), lung cancer (H82, SW1271, H1299, H2228) colon carcinoma (SW480, HCT116) and osteosarcoma (U20S) cell lines.

ASO targeting BX470102.3 (SEQ ID NO: 14) leads to significant cell viability decrease in melanoma (D04, MM415, WM1366, VMM39, Sk-Mel-2, Hs852.T, WM3629, AV5, Sk-Mel-28, WM3211, MM485, WM3060, Sk-Mel-5), trametinib resistant melanoma (D04RM, MM415RM, Sk-Mel-2-RM, WM3629RM), Glioblastoma (U138-MG, T98G, A-172, U87-MG), Neuroblastoma (Sk-N-AS), multiple myeloma (H929, L363), lung cancer (H82, SW1271, H1299) colon carcinoma (SW480, HCT116) and osteosarcoma (U20S) cell lines.

siRNA targeting BX470102.3 (SEQ ID NO: 23) leads to significant cell viability decrease in melanoma (D04, AV5, Sk-Mel-28) cell lines.

ASO targeting AC004540.4 (TRASH) (SEQ ID NO: 15) leads to significant cell viability decrease in melanoma (Hs940.T, AV4, WM3060, Sk-Mel-5, MaMel30), Glioblastoma (U138-MG, T98G, A-172, U87-MG), Neuroblastoma (Sk-N-AS), multiple myeloma (H929), lung cancer (H82, SW1271, H1299, H2228) colon carcinoma (SW480, HCT116, LS174) and osteosarcoma (U20S) cell lines.

siRNA targeting AC004540.4 (SEQ ID NO: 25) leads to significant cell viability decrease in the melanoma AV5 cell line.

ASO targeting RP11-7011.3 (SEQ ID NO: 17) leads to significant cell viability decrease in melanoma (D04, MM415, WM1366, VMM39, Sk-Mel-2, Hs852.T, WM3629, AV5, AV4, AV1, Sk-Mel-28, WM3211, WM3060, Sk-Mel-5, MaMel30), trametinib resistant melanoma (DO4RM, MM415RM, Sk-Mel-2-RM, WM3629RM), Glioblastoma (U138-MG, T98G, A-172, U87-MG), Neuroblastoma (Sk-N-AS), multiple myeloma (H929, L363, XG-1), lung cancer (H82, SW1271, H2228) colon carcinoma (SW480, HCT116) and osteosarcoma (U2OS) cell lines.

ASO targeting RP11-7011.3 (SEQ ID NO: 18) leads to significant cell viability decrease in melanoma (D04, MM415, WM1366, VMM39, Sk-Mel-2, Hs852.T, WM3629, Sk-Mel-28, WM3211, MM485, WM3060, Sk-Mel-5), trametinib resistant melanoma (DO4RM, MM415RM, Sk-Mel-2-RM, WM3629RM), Glioblastoma (U138-MG, T98G, A-172, U87-MG), Neuroblastoma (Sk-N-AS), multiple myeloma (H929, L363), lung cancer (H1299, SW1271) colon carcinoma (SW480, HCT116) and osteosarcoma (U20S) cell lines.

siRNA targeting RP11-7011.3 (SEQ ID NO: 27) leads to significant cell viability decrease in melanoma (D04, AV5, Sk-Mel-28) cell lines.

siRNA targeting RN7SL1 (Pooled SEQ ID NOs: 29,31,33,35) leads to significant cell viability decrease in melanoma (D04, AV5, Sk-Mel-28) cell lines.

ASO targeting ARF-AS1 (SEQ ID NO: 19) leads to significant cell viability decrease in melanoma (D04, MM415, Sk-Mel-2, Sk-Mel-28, MaMel30) and Neuroblastoma (Sk-N-AS) cell lines.

ASO targeting ARF-AS1 (SEQ ID NO: 20) leads to significant cell viability decrease in melanoma (D04, MM415, Sk-Mel-2, Sk-Mel-28) and Neuroblastoma (Sk-N-AS) cell lines.

siRNA targeting ARF-AS1 (SEQ ID NO: 37) leads to significant cell viability decrease in the melanoma cell line D04.

ASO targeting AL157871.4 (SEQ ID NO: 21) leads to significant cell viability decrease in melanoma (D04, MM415, Sk-Mel-2, Sk-Mel-28) and Neuroblastoma (Sk-N-AS) cell lines.

ASO targeting AL157871.4 (SEQ ID NO: 22) leads to significant cell viability decrease in melanoma (D04, MM415, Sk-Mel-2, Sk-Mel-28, MaMel30) and neuroblastoma (Sk-N-AS) cell lines.

siRNA targeting AL157871.4 (SEQ ID NO: 39) leads to significant cell viability decrease in the D04 melanoma cell line.

### Example 4

### In Vitro Results of Additional TRASH-Targeting Oligonucleotides:

### Cell Viability

In vitro treatment with TRASH targeting ASO (SEQ ID NO: 15) with a final concentration of 50 nM in media and an incubation time for 120 hrs lead to significant cell viability decrease in the hepatocellular carcinoma cell line HepG2 and the sarcoma cell line SK-LMS-1. Cell viability was compared to treatment with non-targeting control ASO-treatment. Lipofectamine3000 concentration was 2 ul/ml.

In vitro treatment with TRASH targeting ASO (SEQ ID NO: 41) with a final concentration of 50 nM in media and an incubation time for 120 hrs lead to significant cell viability decrease in the melanoma cell lines D04, MM415, WM1366, VMM39, Sk-Mel-2, Hs852.T, WM3629, AV5, Sk-Mel-28, WM3211, in the hepatocellular carcinoma cell line HepG2 and the sarcoma cell line SK-LMS-1. Cell viability was compared to treatment with non-targeting control ASO-treatment. Lipofectamine3000 concentration was 2 ul/ml.

In vitro treatment with TRASH targeting ASO (SEQ ID NO: 16) with a final concentration of 50 nM in media and an incubation time for 120 hrs lead to significant cell viability decrease in the melanoma cell lines D04, MM415, WM1366, VMM39, Sk-Mel-2, Hs852.T, WM3629, AV5, Sk-Mel-28 and WM3211, in the hepatocellular carcinoma cell line HepG2 and the sarcoma cell line SK-LMS-1. Cell viability was compared to treatment with non-targeting control ASO-treatment. Lipofectamine3000 concentration was 2 ul/ml.

In vitro treatment with TRASH targeting siRNA (SEQ ID NO: 42) with a final concentration of 50 nM in media and an incubation time for 120 hrs lead to significant cell viability decrease in the melanoma cell lines D04, MM415, and Sk-Mel-2. Cell viability was compared to treatment with non-targeting control ASO-treatment. Lipofectamine3000 concentration was 2 ul/ml.

In vitro treatment with TRASH targeting siRNA (SEQ ID NO: 43) with a final concentration of 50 nM in media and an incubation time for 120 hrs lead to significant cell viability decrease in the melanoma cell lines D04, Sk-Mel-2 and WM3629. Cell viability was compared to treatment with non-targeting control ASO-treatment. Lipofectamine3000 concentration was 2 ul/ml.

In vitro treatment with TRASH targeting siRNA (SEQ ID NO: 44) with a final concentration of 50 nM in media and an incubation time for 120 hrs lead to significant cell viability decrease in the melanoma cell lines D04, Sk-Mel-2, MM415 and WM3629. Cell viability was compared to treatment with non-targeting control ASO-treatment. Lipofectamine3000 concentration was 2 ul/ml.

In vitro treatment with TRASH targeting siRNA (SEQ ID NO:45) with a final concentration of 50 nM in media and an incubation time for 120 hrs lead to significant cell viability decrease in the melanoma cell lines D04 and MM415. Cell viability was compared to treatment with non-targeting control ASO-treatment. Lipofectamine3000 concentration was 2 ul/ml.

In vitro treatment with TRASH targeting siRNA (SEQ ID NO:46) with a final concentration of 50 nM in media and an incubation time for 120 hrs lead to significant cell viability decrease in the melanoma cell lines D04 and MM415. Cell viability was compared to treatment with non-targeting control ASO-treatment. Lipofectamine3000 concentration was 2 ul/ml.

In vitro treatment with TRASH targeting ASO (SEQ ID NO:47) and additional Cholesterol modification, with a final concentration of 50 nM in media and an incubation time for 120 hrs lead to significant cell viability decrease in the melanoma cell lines D04, MM415 in the hepatocellular carcinoma cell line HepG2 and the sarcoma cell line SK-LMS-1. Cell viability was compared to treatment with non-targeting control ASO-treatment. Lipofectamine3000 concentration was 2 ul/ml.

In vitro treatment with hnRNPA2/B1 targeting ASO (SEQ ID NO: 48) with a final concentration of 50 nM in media and an incubation time for 120 hrs lead to significant cell viability decrease in the melanoma cell line D04. Cell viability was compared to treatment with non-targeting control ASO-treatment. Lipofectamine3000 concentration was 2 ul/ml.

In vitro treatment with SNX10 targeting ASO (SEQ ID NO: 49) with a final concentration of 50 nM in media and an incubation time for 120 hrs lead to significant cell viability decrease in the melanoma cell line D04. Cell viability was compared to treatment with non-targeting control ASO-treatment. Lipofectamine3000 concentration was 2 ul/ml.

### Intravenous In Vivo Treatment

In 4- to 6-week-old NOD.Cg-Prkdcscid Il2rgtm1Wjl/SzJ mice, D04 cells in 150 1 of PBS and 50 1 of Matrigel were subcutaneously injected on the right and left posterior dorsal flank of 4- to 6-week-old homozygous nude Foxn1nu/Foxn1nu mice (Stock.no 007850). Mice were obtained from JAX®. Tumor size was measured using a digital caliper and the formula 0.5×(length×(width\hat{ }2)) was used to calculate tumor volume. Mice were treated twice a week with 700 μg of (SEQ ID NO: 16) or non-targeting control-ASO. No transfection reagent was co-applied. ASO injections were applied intravenously as tail vein injections. Mice were weighted twice a week and observed for signs of distress or disorder. Mice in the TRASH-ASO treatment group showed significantly reduced tumor growth, when compared to mice in the group that received non-targeting control ASOs.

### Intratumoral In Vivo Treatment

In 4- to 6-week-old NOD.Cg-Prkdcscid Il2rgtm1Wjl/SzJ mice D04, cells in 150 1 of PBS and 501 of Matrigel were subcutaneously injected on the right posterior dorsal flank of 4- to 6-week-old homozygous nude Foxn1nu/Foxn1nu mice (Stock.no 007850). Mice were obtained from JAX®. Tumor size was measured using a digital caliper and the formula 0.5×(length×(width\hat{ }2)) was used to calculate tumor volume. Mice were treated twice a week with 400 μg of (SEQ ID NO: 16) or non-targeting control-ASO. No transfection reagent was co-applied. ASO injections were applied into the tumor mass. Mice were weighted twice a week and observed for signs of distress or disorder. Mice in the TRASH-ASO treatment group showed significantly reduced tumor growth, when compared to mice in the group that received non-targeting control ASOs.

### RNAscope

Representative images of DAPI-, hnRNPA2/B1-, and AC004540.4 (TRASH)-derived fluorescence in untreated D04 melanoma cells show that AC004540.4 (TRASH) transcripts and hnRNPA2/B1 protein are enriched in the nucleus of melanoma cells. (FIG. 5a)

### Flow Cytometry Apoptosis

To confirm activation of apoptosis and cell death in response to TRASH-ASO treatment (SEQ ID NO: 15) D04 cells were either treated with control-ASO or TRASH-ASO (50 nM) for 24 h. The cells were stained with Alexa 488 Annexin V and propidium iodide (PI) (Invitrogen™ Dead Cell Apoptosis Kits with Annexin V for Flow Cytometry Catalog number: V13241). Increased fractions of apoptotic and dead cells in the overall cell population, were seen followed by TRASH-ASO treatment, when compared to Control-ASO treatment. (FIG. 5e)

### MEK-Inhibitor-Induced Upregulation

Relative fold enrichment analysis using RT-qPCR shows that the MEKi trametinib caused dose dependent AC004540.4 (TRASH)-upregulation. D04 cells responded with 3.2-fold upregulation to 20 nM MEKi treatment and 5.4-fold enrichment to 40 nM MEKi treatment. MM415 cells are less vulnerable to MEKi treatment and reacted with 0.31-fold increase (20 nM), respectively 0.36-fold increase of AC004540.4 (TRASH)-expression. Cells were either treated with trametinib (MEKi) or DMSO (control). Treatment period was 72 hours. CT-values were normalized to GAPDH, error bars represent standard deviation. All experiments were performed in triplicates (n=3/group). (FIG. 5b)

AC004540.4 (TRASH)-ASO treatment has a global effect on gene expression and regulates the MAPK and PI3K-AKT signaling cascade

To examine the biomolecular changes upon TRASH-inhibition in melanoma, D04 cells were treated with TRASH-ASOs (SEQ ID NO:15) and Control-ASOs and RNA was extracted and used for RNA-Seq. Differential expression (DE) analysis showed that TRASH-ASOs had a global effect on gene expression. We found that 574 genes were down-regulated, and 493 genes were up-regulated, when compared to Control-ASO treatment (Cut off was >1.5-fold change and FDR <0.05, Table 1). GO term analysis revealed that the top enriched GO term clusters associated with the down-regulated genes were related to “ECM-receptor interaction” and “PI3K-AKT signaling pathway”, while the top enriched GO term clusters associated with the up-regulated genes included the terms “protein tyrosine kinase activity” (GO: 0004713) and “Ras guanyl-nucleotide exchange factor activity” (G00005088) (Table 2). These GO terms consisted of genes encoding growth factors, tyrosine kinases, G protein coupled receptor subunits, and collagen subunits. Scatter plot diagram showing differential gene expression after TRASH-ASO treatment compared to Control-ASO treatment. (cut-off for significance was adjusted p-value <0.05). Data was obtained from RNA-Seq of D04 melanoma cells, treatment period was three days. (FIG. 5c)

These findings suggest that TRASH governs melanoma cell survival and inhibits apoptosis to a stronger extent than its protein binding partner hnRNPA2/B1 and that TRASH may execute its anti-apoptotic functions as a regulator of the MAPK and PI3K-AKT signaling cascade.

### Kinase Activity Profiling Reveals Unique Anti-Apoptotic Features of AC004540.4 (TRASH)-Expression

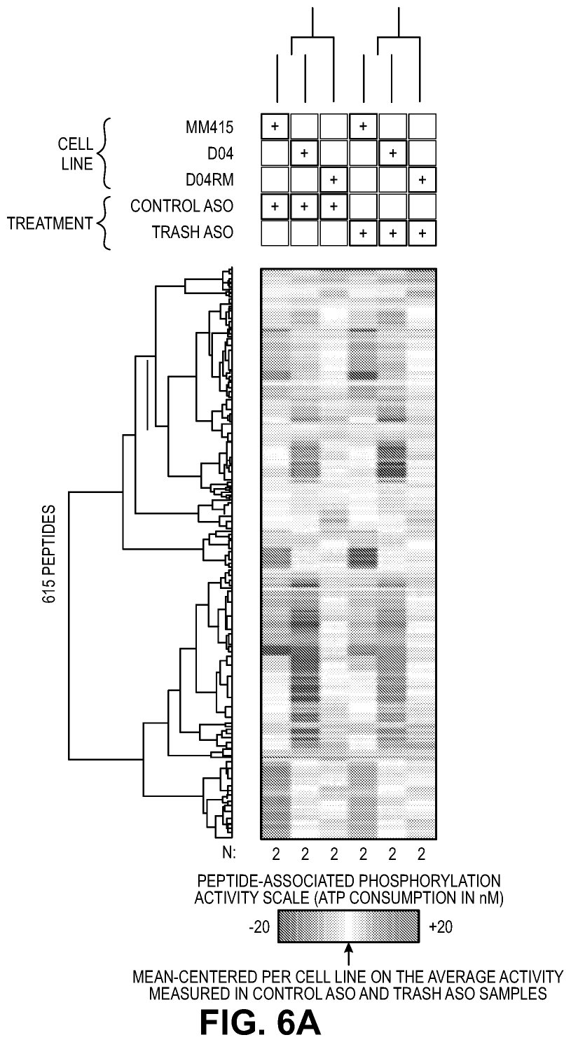

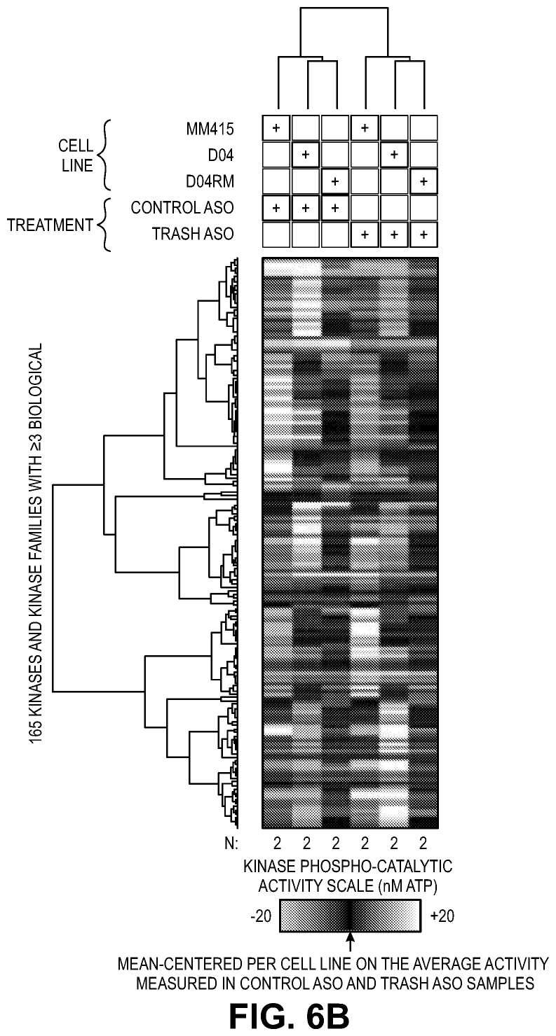

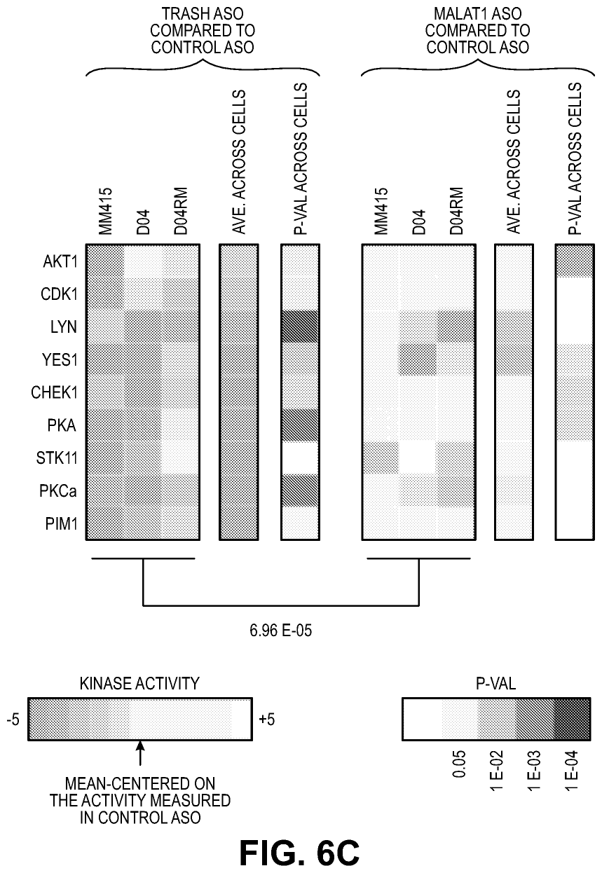

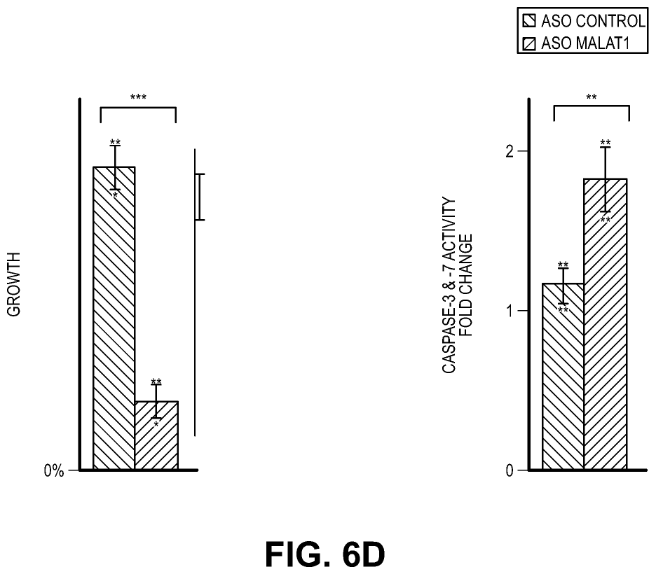

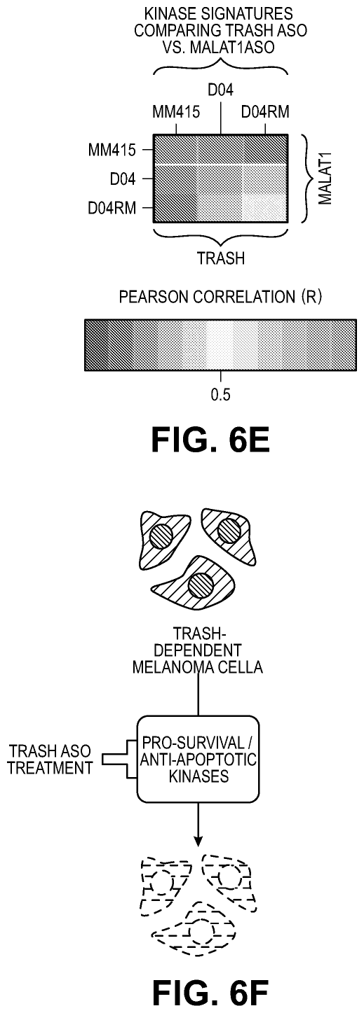

Kinases cover a wide range of apoptosis regulating functions in cancer. Given the findings that TRASH-ASO treatment (SEQ ID NO:15) strongly affects the transcriptional regulation of genes that are related to kinase signaling pathways, we aimed to perform functional profiling of kinase activity shifts triggered by TRASH-inhibition. To do so, we used a kinase activity screening platform39 (named High Throughput Kinase Activity Mapping—HT-KAM) that enables the simultaneous identification of kinase enzymes functional state in cancer cells across a broad range of kinase families (see Methods for details). We generated protein extracts of two versions of the D04 (D04—treatment naive; D04RM—trametinib resistant) and the MM415 melanoma cell-lines, treated with Control-ASOs or TRASH-ASOs. We tested these cell extracts on HT-KAM and performed unsupervised hierarchical clustering of peptide-associated phosphorylation profiles (FIG. 6a) and of kinase activity signatures (FIG. 6b). The changes in kinases' activity upon TRASH-ASO treatment indicate conserved responses across cell-lines, whether kinases are up-regulated or down-regulated (respectively in yellow or blue in FIG. 6b).

Due to the effects of TRASH-ASO treatment on cell viability and apoptosis induction, we focused on kinases with anti-apoptotic functions. We found that the pro-survival/proto-oncogenic kinases AKT1, CDK1, LYN, YES1, CHEK1, PKCA, STK11, PKCa and PIM1 were significantly less active upon TRASH-inhibition (FIG. 6c left panel). These kinases have been reported to regulate the state of caspases and pro-survival pathways including the RAF-MAPK and PI3K-AKT axes.40-47

To further test if these observations are TRASH-ASO treatment specific, we generated MALAT1-ASO treated extracts from the same cell-line models. MALAT1 is a known oncogenic lncRNA in various types of cancer, including melanoma.48,49 MALAT1-ASO treatment reduced cell-growth but displayed a significantly reduced effect on apoptosis induction in comparison to TRASH-inhibition (p=0.002 for 1.6-fold versus 3.0-fold Caspase-3 &-7 activity increase respectively in FIG. 6d and FIG. 3d). Using the HT-KAM platform, we found that the activity of the kinases associated with cell-survival were not down-regulated in MALAT1-ASO treated cells (FIG. 6c right panel), but significantly and specifically down-regulated upon TRASH-ASO treatment (FIG. 6c, p<0.00007; FIG. 6e, kinase signatures of TRASH-, versus MALAT1-ASO treatment). In summary, our data indicate that TRASH-ASO treatment specifically down-regulates the activity of anti-apoptotic kinases and pro-survival signaling pathways in melanoma cells, supporting the potential therapeutic relevance of TRASH-ASO treatment (FIG. 6f).

In comparison to MEKi-treatment, repetitive TRASH-ASO treatment does not lead to early drug-resistance in melanoma

Rescuing cells that survived initial TRASH-ASO (SEQ ID NO:15) and MEKi (trametinib) treatment and providing the rescued cells with a phase of regrowth in drug free media, was followed by repetition of the preceding drug treatment. D04 cells responded with increased vulnerability to 50 nM TRASH-ASO treatment, implying that no drug resistance could be measured. On the other hand, D04 cells that underwent MEKi treatment with 15 nM or 20 nM final concentration responded with significantly less cell-growth inhibition to further MEKi, implying that these cells developed resistance mechanisms that decreased vulnerability to MEKi. Incubation time was 120 hrs, n=3. (FIG. 5d).

TABLE 1

| | | | | |
|---|---|---|---|---|
| ID | Gene.name | log2FoldChange | pvalue | padj |

List of genes that are down-regulated upon TRASH-ASO treatment.

| | | | | |
|---|---|---|---|---|
| ENSG00000137491 | SLCO2B1 | −2.59274 | 1.43E−28 | 1.13E−25 |
| ENSG00000071909 | MYO3B | −1.95637 | 8.37E−05 | 0.001189679 |
| ENSG00000100154 | TTC28 | −1.81477 | 1.22E−09 | 5.86E−08 |
| ENSG00000214456 | PLIN5 | −1.69846 | 0.002436 | 0.019731641 |
| ENSG00000196119 | OR8A1 | −1.673 | 0.002536 | 0.020342008 |
| ENSG00000239268 | AC092691.1 | −1.64048 | 0.000531 | 0.005683777 |
| ENSG00000204334 | ERICH2 | −1.6371 | 0.002101 | 0.017570757 |
| ENSG00000196376 | SLC35F1 | −1.61143 | 8.27E−23 | 3.20E−20 |
| ENSG00000184856 | LINC00308 | −1.6094 | 1.74E−11 | 1.29E−09 |
| ENSG00000182463 | TSHZ2 | −1.59808 | 3.14E−18 | 6.26E−16 |
| ENSG00000148935 | GAS2 | −1.59095 | 0.000169 | 0.002142374 |
| ENSG00000130600 | H19 | −1.56999 | 2.21E−32 | 3.22E−29 |
| ENSG00000237515 | SHISA9 | −1.56986 | 4.34E−10 | 2.34E−08 |
| ENSG00000106278 | PTPRZ1 | −1.56748 | 1.63E−24 | 8.61E−22 |
| ENSG00000204252 | HLA-DOA | −1.56661 | 4.42E−09 | 1.84E−07 |
| ENSG00000114757 | PEX5L | −1.51992 | 4.95E−39 | 1.34E−35 |
| ENSG00000261115 | TMEM178B | −1.51369 | 1.78E−33 | 2.82E−30 |
| ENSG00000174469 | CNTNAP2 | −1.50992 | 0.004949 | 0.033901177 |
| ENSG00000165495 | PKNOX2 | −1.50567 | 2.74E−09 | 1.20E−07 |
| ENSG00000188859 | FAM78B | −1.49819 | 5.53E−25 | 3.00E−22 |
| ENSG00000155974 | GRIP1 | −1.48432 | 1.76E−15 | 2.37E−13 |
| ENSG00000164106 | SCRG1 | −1.47714 | 1.15E−11 | 8.97E−10 |
| ENSG00000125780 | TGM3 | −1.46318 | 3.43E−05 | 0.000555836 |
| ENSG00000163273 | NPPC | −1.46133 | 0.000468 | 0.005136146 |
| ENSG00000176533 | GNG7 | −1.459 | 4.36E−06 | 9.27E−05 |
| ENSG00000256232 | LINC02387 | −1.45291 | 1.83E−06 | 4.34E−05 |
| ENSG00000167617 | CDC42EP5 | −1.42861 | 1.02E−05 | 0.000192688 |
| ENSG00000144668 | ITGA9 | −1.41719 | 1.52E−13 | 1.58E−11 |
| ENSG00000127954 | STEAP4 | −1.40458 | 3.03E−19 | 6.84E−17 |
| ENSG00000198774 | RASSF9 | −1.40194 | 6.09E−12 | 4.93E−10 |
| ENSG00000065717 | TLE2 | −1.38255 | 0.000253 | 0.003053978 |
| ENSG00000266296 | ARIH2P1 | −1.37762 | 0.000204 | 0.002530044 |
| ENSG00000185924 | RTN4RL1 | −1.34556 | 1.48E−06 | 3.62E−05 |
| ENSG00000036565 | SLC18A1 | −1.33718 | 9.01E−15 | 1.08E−12 |
| ENSG00000257252 | AC124947.1 | −1.33042 | 8.84E−05 | 0.001242963 |
| ENSG00000223802 | CERS1 | −1.32574 | 0.000347 | 0.003990157 |
| ENSG00000225649 | AC064875.1 | −1.32525 | 1.45E−21 | 4.83E−19 |
| ENSG00000213606 | AKR1B10P1 | −1.31707 | 4.40E−07 | 1.20E−05 |
| ENSG00000223764 | AL645608.1 | −1.30979 | 3.43E−09 | 1.47E−07 |
| ENSG00000255864 | NA | −1.30375 | 0.003279 | 0.024952707 |
| ENSG00000231683 | AL033397.1 | −1.28997 | 0.000774 | 0.007770437 |
| ENSG00000170153 | RNF150 | −1.28344 | 2.68E−20 | 7.47E−18 |
| ENSG00000249226 | SUCLG2P4 | −1.27377 | 0.00607 | 0.040019666 |
| ENSG00000171408 | PDE7B | −1.27139 | 7.37E−17 | 1.20E−14 |
| ENSG00000145824 | CXCL14 | −1.26291 | 3.93E−29 | 3.92E−26 |
| ENSG00000113361 | CDH6 | −1.25809 | 1.37E−11 | 1.05E−09 |
| ENSG00000116117 | PARD3B | −1.25733 | 1.75E−13 | 1.80E−11 |
| ENSG00000091129 | NRCAM | −1.25597 | 1.81E−15 | 2.40E−13 |
| ENSG00000179772 | FOXS1 | −1.24693 | 0.00582 | 0.038679779 |
| ENSG00000228651 | AC074327.1 | −1.24201 | 1.02E−08 | 3.88E−07 |
| ENSG00000278616 | BEND3P3 | −1.24019 | 8.12E−05 | 0.0011626 |
| ENSG00000135925 | WNT10A | −1.23715 | 3.27E−06 | 7.21E−05 |
| ENSG00000260412 | AL353746.1 | −1.23235 | 0.00023 | 0.002819982 |
| ENSG00000151322 | NPAS3 | −1.22502 | 3.53E−05 | 0.000569027 |
| ENSG00000271216 | LINC01050 | −1.2229 | 2.55E−05 | 0.000428671 |
| ENSG00000171864 | PRND | −1.21506 | 0.007983 | 0.049623263 |
| ENSG00000148848 | ADAM12 | −1.21482 | 9.19E−12 | 7.20E−10 |
| ENSG00000108846 | ABCC3 | −1.2128 | 6.79E−29 | 6.13E−26 |
| ENSG00000102996 | MMP15 | −1.21185 | 6.70E−31 | 7.47E−28 |
| ENSG00000183580 | FBXL7 | −1.20853 | 2.65E−08 | 9.35E−07 |
| ENSG00000134533 | RERG | −1.2041 | 1.50E−23 | 6.32E−21 |
| ENSG00000205517 | RGL3 | −1.20067 | 2.04E−12 | 1.81E−10 |
| ENSG00000250995 | AL391280.1 | −1.1929 | 0.000353 | 0.004051833 |
| ENSG00000237653 | AC026320.2 | −1.19234 | 0.000114 | 0.001537941 |
| ENSG00000196776 | CD47 | −1.18982 | 1.72E−36 | 3.62E−33 |
| ENSG00000280355 | AL132656.4 | −1.17588 | 0.004722 | 0.032715724 |
| ENSG00000115596 | WNT6 | −1.17314 | 2.25E−09 | 1.00E−07 |
| ENSG00000170915 | PAQR8 | −1.16477 | 8.70E−15 | 1.05E−12 |
| ENSG00000111319 | SCNN1A | −1.16331 | 0.00189 | 0.016111329 |
| ENSG00000187955 | COL14A1 | −1.16216 | 7.59E−06 | 0.000149681 |
| ENSG00000184371 | CSF1 | −1.15814 | 7.16E−17 | 1.18E−14 |
| ENSG00000151632 | AKR1C2 | −1.15749 | 1.22E−19 | 3.00E−17 |
| ENSG00000100239 | PPP6R2 | −1.15491 | 3.58E−36 | 6.79E−33 |
| ENSG00000183682 | BMP8A | −1.1354 | 0.000608 | 0.006341081 |
| ENSG00000171873 | ADRA1D | −1.12926 | 0.003176 | 0.024333664 |
| ENSG00000181444 | ZNF467 | −1.12657 | 2.70E−06 | 6.09E−05 |
| ENSG00000171557 | FGG | −1.12275 | 2.40E−19 | 5.55E−17 |
| ENSG00000134532 | SOX5 | −1.11563 | 3.73E−17 | 6.37E−15 |
| ENSG00000080573 | COL5A3 | −1.10808 | 2.39E−06 | 5.50E−05 |
| ENSG00000135324 | MRAP2 | −1.10629 | 0.004617 | 0.032122369 |
| ENSG00000089356 | FXYD3 | −1.10201 | 1.95E−09 | 8.79E−08 |
| ENSG00000105967 | TFEC | −1.09855 | 0.003289 | 0.024994517 |
| ENSG00000272502 | AC104958.2 | −1.0985 | 0.000511 | 0.005513125 |
| ENSG00000248587 | GDNF-AS1 | −1.09821 | 0.001376 | 0.012487738 |
| ENSG00000242808 | SOX2-OT | −1.09805 | 0.000645 | 0.006643746 |
| ENSG00000196735 | HLA-DQA1 | −1.09803 | 2.65E−15 | 3.44E−13 |
| ENSG00000102313 | ITIH6 | −1.09802 | 0.000829 | 0.008250813 |
| ENSG00000135414 | GDF11 | −1.09604 | 3.47E−31 | 4.11E−28 |
| ENSG00000241168 | AC128685.1 | −1.09378 | 0.000867 | 0.008549537 |
| ENSG00000144481 | TRPM8 | −1.09349 | 1.15E−12 | 1.07E−10 |
| ENSG00000230490 | AL139383.1 | −1.08952 | 9.04E−07 | 2.29E−05 |
| ENSG00000204248 | COL11A2 | −1.08848 | 3.01E−08 | 1.04E−06 |
| ENSG00000149564 | ESAM | −1.086 | 0.007588 | 0.047658018 |
| ENSG00000174482 | LINGO2 | −1.08599 | 2.02E−06 | 4.72E−05 |
| ENSG00000227471 | AKR1B15 | −1.08295 | 2.52E−08 | 8.96E−07 |
| ENSG00000182050 | MGAT4C | −1.08073 | 7.29E−07 | 1.89E−05 |
| ENSG00000244694 | PTCHD4 | −1.08063 | 3.97E−15 | 5.09E−13 |
| ENSG00000204655 | MOG | −1.07526 | 0.006692 | 0.043174454 |
| ENSG00000185305 | ARL15 | −1.07422 | 3.73E−12 | 3.21E−10 |
| ENSG00000154162 | CDH12 | −1.07121 | 3.99E−11 | 2.73E−09 |
| ENSG00000169760 | NLGN1 | −1.06965 | 8.42E−17 | 1.35E−14 |
| ENSG00000021826 | CPS1 | −1.06652 | 3.24E−14 | 3.59E−12 |
| ENSG00000163638 | ADAMTS9 | −1.06624 | 4.38E−13 | 4.32E−11 |
| ENSG00000116981 | NT5C1A | −1.06503 | 0.002495 | 0.020099296 |
| ENSG00000259803 | SLC22A31 | −1.06459 | 1.70E−05 | 0.000300298 |
| ENSG00000283563 | AC098650.1 | −1.06287 | 0.00517 | 0.035042492 |
| ENSG00000078018 | MAP2 | −1.06068 | 1.25E−23 | 5.64E−21 |
| ENSG00000177570 | SAMD12 | −1.06062 | 9.04E−13 | 8.53E−11 |
| ENSG00000273259 | AL049839.2 | −1.05974 | 0.000106 | 0.001449506 |
| ENSG00000091972 | CD200 | −1.05153 | 6.96E−10 | 3.58E−08 |
| ENSG00000165125 | TRPV6 | −1.04961 | 3.25E−08 | 1.11E−06 |
| ENSG00000234352 | AC009264.1 | −1.04818 | 0.002107 | 0.017617548 |
| ENSG00000138639 | ARHGAP24 | −1.04723 | 4.26E−07 | 1.17E−05 |
| ENSG00000184005 | ST6GALNAC3 | −1.04577 | 1.43E−07 | 4.27E−06 |
| ENSG00000178662 | CSRNP3 | −1.04503 | 0.002115 | 0.017670936 |
| ENSG00000204262 | COL5A2 | −1.04378 | 5.42E−23 | 2.14E−20 |
| ENSG00000104321 | TRPA1 | −1.04327 | 8.15E−08 | 2.59E−06 |
| ENSG00000197892 | KIF13B | −1.0421 | 7.75E−28 | 5.65E−25 |
| ENSG00000060718 | COL11A1 | −1.04016 | 4.17E−18 | 8.06E−16 |
| ENSG00000117266 | CDK18 | −1.03668 | 2.99E−08 | 1.04E−06 |
| ENSG00000171227 | TMEM37 | −1.03519 | 0.002153 | 0.017917961 |
| ENSG00000185532 | PRKG1 | −1.03073 | 5.33E−07 | 1.42E−05 |
| ENSG00000166292 | TMEM100 | −1.02975 | 9.09E−16 | 1.29E−13 |
| ENSG00000144278 | GALNT13 | −1.02882 | 9.06E−05 | 0.001268043 |
| ENSG00000183625 | CCR3 | −1.0285 | 3.11E−05 | 0.000513654 |
| ENSG00000176463 | SLCO3A1 | −1.028 | 2.66E−22 | 9.71E−20 |
| ENSG00000153902 | LGI4 | −1.02296 | 0.003048 | 0.023644413 |
| ENSG00000170961 | HAS2 | −1.02244 | 3.58E−05 | 0.000575118 |
| ENSG00000154274 | C4orf19 | −1.02075 | 7.25E−05 | 0.001055186 |
| ENSG00000113721 | PDGFRB | −1.01777 | 1.18E−17 | 2.17E−15 |
| ENSG00000160145 | KALRN | −1.01664 | 9.58E−21 | 2.88E−18 |
| ENSG00000100346 | CACNA1I | −1.01424 | 2.54E−07 | 7.24E−06 |
| ENSG00000169744 | LDB2 | −1.0138 | 1.26E−09 | 6.05E−08 |
| ENSG00000152926 | ZNF117 | −1.01041 | 0.000144 | 0.001878874 |
| ENSG00000106605 | BLVRA | −1.00941 | 6.42E−16 | 9.22E−14 |
| ENSG00000156298 | TSPAN7 | −1.00771 | 0.007757 | 0.048526251 |
| ENSG00000243679 | AC018638.5 | −1.0053 | 0.002234 | 0.018453963 |
| ENSG00000177301 | KCNA2 | −1.0031 | 0.00042 | 0.004662913 |
| ENSG00000250451 | HOXC-AS1 | −1.0019 | 0.002393 | 0.019457151 |
| ENSG00000150760 | DOCK1 | −1.00182 | 4.14E−29 | 3.92E−26 |
| ENSG00000155966 | AFF2 | −1.00125 | 1.36E−07 | 4.10E−06 |
| ENSG00000186480 | INSIG1 | −1.00074 | 1.16E−28 | 9.56E−26 |
| ENSG00000162512 | SDC3 | −0.99828 | 1.97E−15 | 2.59E−13 |
| ENSG00000118322 | ATP10B | −0.99655 | 1.22E−14 | 1.42E−12 |
| ENSG00000235823 | OLMALINC | −0.9945 | 7.35E−05 | 0.001067279 |
| ENSG00000231389 | HLA-DPA1 | −0.98561 | 2.71E−28 | 2.05E−25 |
| ENSG00000145362 | ANK2 | −0.98437 | 7.08E−18 | 1.33E−15 |
| ENSG00000215283 | HMGB3P24 | −0.98329 | 6.97E−10 | 3.58E−08 |
| ENSG00000172572 | PDE3A | −0.98307 | 1.81E−08 | 6.57E−07 |
| ENSG00000065413 | ANKRD44 | −0.98287 | 1.12E−07 | 3.46E−06 |
| ENSG00000164330 | EBF1 | −0.98179 | 5.87E−13 | 5.68E−11 |
| ENSG00000281881 | NA | −0.98108 | 4.98E−06 | 0.000104434 |
| ENSG00000171502 | COL24A1 | −0.9807 | 3.57E−07 | 9.96E−06 |
| ENSG00000158887 | MPZ | −0.97782 | 5.50E−27 | 3.47E−24 |
| ENSG00000163618 | CADPS | −0.97747 | 4.21E−23 | 1.70E−20 |
| ENSG00000099194 | SCD | −0.97709 | 6.58E−22 | 2.23E−19 |
| ENSG00000171564 | FGB | −0.96588 | 2.94E−07 | 8.28E−06 |
| ENSG00000084636 | COL16A1 | −0.96522 | 1.55E−15 | 2.10E−13 |
| ENSG00000125931 | CITED1 | −0.96518 | 6.91E−14 | 7.49E−12 |
| ENSG00000114948 | ADAM23 | −0.95958 | 7.72E−20 | 1.98E−17 |
| ENSG00000141744 | PNMT | −0.95608 | 0.000158 | 0.002025149 |
| ENSG00000163513 | TGFBR2 | −0.95567 | 1.12E−26 | 6.83E−24 |
| ENSG00000260001 | TGFBR3L | −0.95414 | 0.005412 | 0.036390616 |
| ENSG00000064999 | ANKS1A | −0.95115 | 5.95E−17 | 9.99E−15 |
| ENSG00000179532 | DNHD1 | −0.94858 | 9.37E−10 | 4.67E−08 |
| ENSG00000196136 | SERPINA3 | −0.94492 | 3.01E−12 | 2.63E−10 |
| ENSG00000232774 | AL355916.1 | −0.94469 | 1.02E−20 | 2.97E−18 |
| ENSG00000146250 | PRSS35 | −0.94323 | 0.000325 | 0.003781602 |
| ENSG00000164125 | FAM198B | −0.94321 | 1.83E−27 | 1.25E−24 |
| ENSG00000271270 | TMCC1-AS1 | −0.94256 | 4.67E−08 | 1.56E−06 |
| ENSG00000185565 | LSAMP | −0.94199 | 5.67E−16 | 8.27E−14 |
| ENSG00000253910 | PCDHGB2 | −0.9415 | 0.002425 | 0.019662527 |
| ENSG00000198597 | ZNF536 | −0.94019 | 2.25E−13 | 2.28E−11 |
| ENSG00000133519 | ZDHHC8P1 | −0.93699 | 0.000701 | 0.007117719 |
| ENSG00000121068 | TBX2 | −0.93622 | 6.01E−21 | 1.87E−18 |
| ENSG00000169418 | NPR1 | −0.93597 | 3.54E−05 | 0.000570212 |
| ENSG00000158270 | COLEC12 | −0.93578 | 1.45E−05 | 0.00026297 |
| ENSG00000105894 | PTN | −0.93499 | 9.15E−08 | 2.87E−06 |
| ENSG00000129009 | ISLR | −0.93395 | 2.90E−09 | 1.26E−07 |
| ENSG00000281490 | CICP14 | −0.93377 | 6.20E−07 | 1.63E−05 |
| ENSG00000279400 | AC008957.3 | −0.9335 | 0.000426 | 0.004719696 |
| ENSG00000261468 | AC096921.2 | −0.93054 | 0.004858 | 0.03340762 |
| ENSG00000105767 | CADM4 | −0.92958 | 7.92E−29 | 6.83E−26 |
| ENSG00000262454 | MIR193BHG | −0.92877 | 0.00227 | 0.018659523 |
| ENSG00000267280 | TBX2-AS1 | −0.92858 | 1.56E−11 | 1.18E−09 |
| ENSG00000116132 | PRRX1 | −0.92821 | 6.17E−09 | 2.48E−07 |
| ENSG00000198074 | AKR1B10 | −0.92451 | 4.99E−22 | 1.75E−19 |
| ENSG00000137727 | ARHGAP20 | −0.92281 | 8.02E−05 | 0.00115 |
| ENSG00000149403 | GRIK4 | −0.92124 | 0.000655 | 0.006724913 |
| ENSG00000164418 | GRIK2 | −0.91732 | 1.45E−11 | 1.10E−09 |
| ENSG00000082196 | C1QTNF3 | −0.91728 | 4.74E−06 | 0.000100304 |
| ENSG00000169432 | SCN9A | −0.91655 | 4.60E−15 | 5.78E−13 |
| ENSG00000134108 | ARL8B | −0.912 | 4.69E−22 | 1.68E−19 |
| ENSG00000125848 | FLRT3 | −0.91188 | 1.48E−22 | 5.60E−20 |
| ENSG00000250658 | AC097652.1 | −0.90896 | 0.006612 | 0.042817232 |
| ENSG00000137285 | TUBB2B | −0.90539 | 2.15E−10 | 1.26E−08 |
| ENSG00000163554 | SPTA1 | −0.90282 | 1.61E−06 | 3.87E−05 |
| ENSG00000156475 | PPP2R2B | −0.90029 | 1.02E−09 | 5.00E−08 |
| ENSG00000178860 | MSC | −0.89782 | 3.04E−24 | 1.44E−21 |
| ENSG00000105556 | MIER2 | −0.89672 | 5.81E−11 | 3.77E−09 |
| ENSG00000081189 | MEF2C | −0.89393 | 5.13E−21 | 1.62E−18 |
| ENSG00000083067 | TRPM3 | −0.89001 | 1.87E−10 | 1.11E−08 |
| ENSG00000162630 | B3GALT2 | −0.89 | 2.33E−11 | 1.69E−09 |
| ENSG00000221817 | PPP3CB-AS1 | −0.88744 | 6.50E−05 | 0.000965227 |
| ENSG00000101638 | ST8SIA5 | −0.882 | 9.78E−06 | 0.000185969 |
| ENSG00000133048 | CHI3L1 | −0.87844 | 4.75E−19 | 1.02E−16 |
| ENSG00000248079 | DPH6-AS1 | −0.8774 | 3.14E−05 | 0.000516851 |
| ENSG00000172164 | SNTB1 | −0.87709 | 3.23E−27 | 2.11E−24 |
| ENSG00000082482 | KCNK2 | −0.87539 | 0.002731 | 0.021578122 |
| ENSG00000164176 | EDIL3 | −0.87187 | 3.52E−09 | 1.50E−07 |
| ENSG00000196083 | IL1RAP | −0.87119 | 3.31E−20 | 8.72E−18 |
| ENSG00000258655 | ARHGAP5-AS1 | −0.87077 | 2.07E−11 | 1.52E−09 |
| ENSG00000140285 | FGF7 | −0.86661 | 4.96E−10 | 2.62E−08 |
| ENSG00000162687 | KCNT2 | −0.86596 | 0.003821 | 0.027929486 |
| ENSG00000167680 | SEMA6B | −0.86474 | 6.82E−24 | 3.15E−21 |
| ENSG00000151490 | PTPRO | −0.86166 | 7.01E−12 | 5.60E−10 |
| ENSG00000106302 | HYAL4 | −0.86142 | 1.13E−05 | 0.000210994 |
| ENSG00000138376 | BARD1 | −0.85724 | 3.00E−18 | 6.06E−16 |
| ENSG00000236651 | DLX2-AS1 | −0.85686 | 0.001927 | 0.016380173 |
| ENSG00000162745 | OLFML2B | −0.85506 | 0.000632 | 0.006540012 |
| ENSG00000168874 | ATOH8 | −0.85504 | 4.22E−10 | 2.29E−08 |
| ENSG00000203883 | SOX18 | −0.85418 | 0.001192 | 0.01112841 |
| ENSG00000169851 | PCDH7 | −0.85314 | 1.29E−23 | 5.68E−21 |
| ENSG00000166448 | TMEM130 | −0.85307 | 1.74E−07 | 5.13E−06 |
| ENSG00000149212 | SESN3 | −0.85186 | 4.21E−18 | 8.07E−16 |
| ENSG00000244342 | LINC00698 | −0.85013 | 0.004333 | 0.030686931 |
| ENSG00000114200 | BCHE | −0.84938 | 2.00E−11 | 1.47E−09 |
| ENSG00000147434 | CHRNA6 | −0.84929 | 5.07E−05 | 0.000770826 |
| ENSG00000169855 | ROBO1 | −0.84624 | 1.27E−17 | 2.31E−15 |
| ENSG00000112782 | CLIC5 | −0.84527 | 0.005444 | 0.036522616 |
| ENSG00000164756 | SLC30A8 | −0.8442 | 0.000234 | 0.002855168 |
| ENSG00000152127 | MGAT5 | −0.84393 | 4.43E−19 | 9.69E−17 |
| ENSG00000131094 | C1QL1 | −0.84297 | 9.35E−05 | 0.001299079 |
| ENSG00000112559 | MDFI | −0.84207 | 5.61E−06 | 0.000115494 |
| ENSG00000103528 | SYT17 | −0.84191 | 9.43E−07 | 2.38E−05 |
| ENSG00000109339 | MAPK10 | −0.83897 | 0.001337 | 0.012176905 |
| ENSG00000186376 | ZNF75D | −0.83886 | 6.98E−07 | 1.82E−05 |
| ENSG00000249669 | CARMN | −0.83551 | 3.70E−10 | 2.05E−08 |
| ENSG00000183615 | FAM167B | −0.83491 | 0.006451 | 0.041988732 |
| ENSG00000181035 | SLC25A42 | −0.83329 | 0.001234 | 0.011443029 |
| ENSG00000104324 | CPQ | −0.83308 | 1.49E−09 | 6.92E−08 |
| ENSG00000189157 | FAM47E | −0.83097 | 0.003693 | 0.027194946 |
| ENSG00000102287 | GABRE | −0.82978 | 4.29E−13 | 4.26E−11 |
| ENSG00000079931 | MOXD1 | −0.8296 | 1.11E−19 | 2.78E−17 |
| ENSG00000127863 | TNFRSF19 | −0.82829 | 5.36E−20 | 1.39E−17 |
| ENSG00000114646 | CSPG5 | −0.82797 | 9.72E−05 | 0.001346433 |
| ENSG00000213614 | HEXA | −0.82781 | 2.44E−22 | 9.05E−20 |
| ENSG00000234535 | AL161719.1 | −0.82742 | 0.007087 | 0.045108485 |
| ENSG00000097033 | SH3GLB1 | −0.82679 | 1.71E−20 | 4.83E−18 |
| ENSG00000165272 | AQP3 | −0.82505 | 7.14E−07 | 1.85E−05 |
| ENSG00000123684 | LPGAT1 | −0.82405 | 2.00E−24 | 9.95E−22 |
| ENSG00000116991 | SIPA1L2 | −0.82336 | 8.58E−14 | 9.19E−12 |
| ENSG00000159307 | SCUBE1 | −0.81869 | 2.17E−11 | 1.59E−09 |
| ENSG00000072310 | SREBF1 | −0.81558 | 6.03E−14 | 6.57E−12 |
| ENSG00000116774 | OLFML3 | −0.81477 | 1.24E−08 | 4.68E−07 |
| ENSG00000260289 | AC093515.1 | −0.81389 | 0.000983 | 0.009515747 |
| ENSG00000256433 | AC005840.2 | −0.81141 | 0.007622 | 0.047822298 |
| ENSG00000163823 | CCR1 | −0.8105 | 0.006612 | 0.042817232 |
| ENSG00000232353 | AC026320.1 | −0.81018 | 0.001412 | 0.012727888 |
| ENSG00000184304 | PRKD1 | −0.80889 | 3.45E−11 | 2.40E−09 |
| ENSG00000108352 | RAPGEFL1 | −0.80744 | 3.24E−05 | 0.000530316 |
| ENSG00000166402 | TUB | −0.80674 | 2.30E−14 | 2.58E−12 |
| ENSG00000120925 | RNF170 | −0.80611 | 8.45E−09 | 3.30E−07 |
| ENSG00000179104 | TMTC2 | −0.80599 | 3.12E−20 | 8.44E−18 |
| ENSG00000112699 | GMDS | −0.80551 | 5.15E−09 | 2.11E−07 |
| ENSG00000123739 | PLA2G12A | −0.80439 | 1.63E−17 | 2.91E−15 |
| ENSG00000100084 | HIRA | −0.80401 | 2.44E−11 | 1.75E−09 |
| ENSG00000279382 | AC018665.1 | −0.80383 | 2.96E−05 | 0.000491018 |
| ENSG00000187634 | SAMD11 | −0.80025 | 9.96E−13 | 9.34E−11 |
| ENSG00000065361 | ERBB3 | −0.79939 | 1.31E−20 | 3.77E−18 |
| ENSG00000157680 | DGKI | −0.79915 | 6.86E−07 | 1.79E−05 |
| ENSG00000170381 | SEMA3E | −0.79837 | 8.48E−06 | 0.000165423 |
| ENSG00000168675 | LDLRAD4 | −0.79819 | 2.42E−11 | 1.74E−09 |
| ENSG00000135363 | LMO2 | −0.79791 | 5.48E−07 | 1.46E−05 |
| ENSG00000168952 | STXBP6 | −0.79536 | 5.45E−08 | 1.79E−06 |
| ENSG00000204272 | NBDY | −0.78925 | 9.87E−11 | 6.24E−09 |
| ENSG00000184564 | SLITRK6 | −0.78815 | 2.47E−11 | 1.77E−09 |
| ENSG00000144810 | COL8A1 | −0.78804 | 9.52E−18 | 1.77E−15 |
| ENSG00000066382 | MPPED2 | −0.78768 | 0.005912 | 0.039181271 |
| ENSG00000164056 | SPRY1 | −0.78616 | 6.54E−08 | 2.11E−06 |
| ENSG00000278910 | BANCR | −0.78517 | 1.08E−07 | 3.35E−06 |
| ENSG00000172508 | CARNS1 | −0.78365 | 0.005236 | 0.035408734 |
| ENSG00000071991 | CDH19 | −0.78153 | 7.05E−13 | 6.68E−11 |
| ENSG00000239282 | CASTOR1 | −0.77922 | 0.004563 | 0.031889425 |
| ENSG00000146147 | MLIP | −0.77917 | 0.000349 | 0.004019244 |
| ENSG00000198753 | PLXNB3 | −0.779 | 2.83E−07 | 7.99E−06 |
| ENSG00000162407 | PLPP3 | −0.77808 | 1.24E−15 | 1.71E−13 |
| ENSG00000143341 | HMCN1 | −0.77742 | 7.93E−08 | 2.53E−06 |
| ENSG00000102038 | SMARCA1 | −0.77569 | 2.02E−12 | 1.80E−10 |
| ENSG00000056998 | GYG2 | −0.77436 | 1.08E−11 | 8.40E−10 |
| ENSG00000279717 | AC005336.3 | −0.77429 | 0.003861 | 0.028155357 |
| ENSG00000183773 | AIFM3 | −0.77262 | 9.21E−06 | 0.000176979 |
| ENSG00000243224 | AC006252.1 | −0.7723 | 0.002444 | 0.01978047 |
| ENSG00000141376 | BCAS3 | −0.77121 | 1.21E−16 | 1.90E−14 |
| ENSG00000143171 | RXRG | −0.76861 | 0.000739 | 0.007468754 |
| ENSG00000106123 | EPHB6 | −0.76699 | 1.87E−13 | 1.92E−11 |
| ENSG00000160307 | S100B | −0.76541 | 9.50E−16 | 1.33E−13 |
| ENSG00000154310 | TNIK | −0.76474 | 9.79E−16 | 1.36E−13 |
| ENSG00000021645 | NRXN3 | −0.76472 | 1.03E−08 | 3.93E−07 |
| ENSG00000090971 | NAT14 | −0.76469 | 5.09E−12 | 4.18E−10 |
| ENSG00000144218 | AFF3 | −0.76428 | 0.000255 | 0.00307443 |
| ENSG00000164946 | FREM1 | −0.76384 | 3.33E−12 | 2.88E−10 |
| ENSG00000007174 | DNAH9 | −0.76291 | 2.64E−06 | 5.98E−05 |
| ENSG00000185561 | TLCD2 | −0.7628 | 3.55E−09 | 1.50E−07 |
| ENSG00000153944 | MSI2 | −0.7628 | 1.35E−19 | 3.27E−17 |
| ENSG00000148655 | LRMDA | −0.76091 | 1.49E−06 | 3.62E−05 |
| ENSG00000176406 | RIMS2 | −0.76017 | 7.99E−06 | 0.000156993 |
| ENSG00000167588 | GPD1 | −0.7585 | 2.24E−07 | 6.46E−06 |
| ENSG00000176049 | JAKMIP2 | −0.7585 | 4.95E−17 | 8.38E−15 |
| ENSG00000156427 | FGF18 | −0.75786 | 0.000381 | 0.004298311 |
| ENSG00000175538 | KCNE3 | −0.7558 | 0.003744 | 0.027481411 |
| ENSG00000183098 | GPC6 | −0.75536 | 8.18E−06 | 0.00016001 |
| ENSG00000112561 | TFEB | −0.75502 | 1.11E−06 | 2.76E−05 |
| ENSG00000033327 | GAB2 | −0.7547 | 3.09E−06 | 6.87E−05 |
| ENSG00000169933 | FRMPD4 | −0.75304 | 7.28E−05 | 0.00105688 |
| ENSG00000185189 | NRBP2 | −0.75225 | 8.01E−12 | 6.33E−10 |
| ENSG00000196139 | AKR1C3 | −0.75127 | 1.50E−10 | 9.05E−09 |
| ENSG00000101384 | JAG1 | −0.75101 | 2.41E−18 | 4.97E−16 |
| ENSG00000130150 | MOSPD2 | −0.75081 | 1.18E−16 | 1.87E−14 |
| ENSG00000117643 | MAN1C1 | −0.75003 | 1.82E−05 | 0.000318073 |
| ENSG00000116741 | RGS2 | −0.74831 | 6.68E−13 | 6.36E−11 |
| ENSG00000123405 | NFE2 | −0.74703 | 0.001024 | 0.009848745 |
| ENSG00000165029 | ABCA1 | −0.74622 | 2.92E−18 | 5.96E−16 |
| ENSG00000143167 | GPA33 | −0.74587 | 0.001309 | 0.011973668 |
| ENSG00000183196 | CHST6 | −0.74518 | 4.71E−12 | 3.90E−10 |
| ENSG00000147588 | PMP2 | −0.74408 | 8.72E−06 | 0.000169029 |
| ENSG00000226043 | AP000561.1 | −0.74251 | 0.004843 | 0.033323943 |
| ENSG00000185885 | IFITM1 | −0.742 | 0.000136 | 0.001787648 |
| ENSG00000116194 | ANGPTL1 | −0.74065 | 0.002773 | 0.021861525 |
| ENSG00000254911 | SCARNA9 | −0.74059 | 4.40E−08 | 1.48E−06 |
| ENSG00000226328 | NUP50-AS1 | −0.73845 | 4.50E−07 | 1.23E−05 |
| ENSG00000260035 | AC051619.8 | −0.73579 | 0.0012 | 0.011181936 |
| ENSG00000124766 | SOX4 | −0.73574 | 5.70E−11 | 3.72E−09 |
| ENSG00000183826 | BTBD9 | −0.7347 | 1.48E−09 | 6.89E−08 |
| ENSG00000111728 | ST8SIA1 | −0.73441 | 0.000211 | 0.002612202 |
| ENSG00000138795 | LEF1 | −0.73345 | 1.31E−08 | 4.88E−07 |
| ENSG00000112419 | PHACTR2 | −0.73312 | 2.33E−11 | 1.69E−09 |
| ENSG00000091428 | RAPGEF4 | −0.73257 | 0.000387 | 0.004357826 |
| ENSG00000182168 | UNC5C | −0.73235 | 2.53E−10 | 1.46E−08 |
| ENSG00000188738 | FSIP2 | −0.73208 | 3.59E−05 | 0.000575889 |
| ENSG00000082293 | COL19A1 | −0.73205 | 2.49E−08 | 8.88E−07 |
| ENSG00000156103 | MMP16 | −0.73123 | 2.32E−17 | 4.03E−15 |
| ENSG00000243244 | STON1 | −0.72972 | 5.07E−06 | 0.000106064 |
| ENSG00000092758 | COL9A3 | −0.72667 | 3.53E−09 | 1.50E−07 |
| ENSG00000251574 | AC099520.1 | −0.72621 | 0.006176 | 0.040617395 |
| ENSG00000130702 | LAMA5 | −0.7258 | 4.03E−06 | 8.67E−05 |
| ENSG00000075213 | SEMA3A | −0.72565 | 8.43E−10 | 4.24E−08 |
| ENSG00000137266 | SLC22A23 | −0.72474 | 1.84E−08 | 6.66E−07 |
| ENSG00000123700 | KCNJ2 | −0.72471 | 0.000135 | 0.001775122 |
| ENSG00000106078 | COBL | −0.72462 | 0.006009 | 0.039700517 |
| ENSG00000170624 | SGCD | −0.72337 | 6.90E−05 | 0.001014739 |
| ENSG00000185432 | METTL7A | −0.72256 | 4.76E−08 | 1.59E−06 |
| ENSG00000197959 | DNM3 | −0.72102 | 1.87E−09 | 8.46E−08 |
| ENSG00000128655 | PDE11A | −0.72097 | 1.01E−05 | 0.000191267 |
| ENSG00000130338 | TULP4 | −0.71932 | 1.10E−05 | 0.000205503 |
| ENSG00000253379 | NA | −0.71904 | 7.67E−05 | 0.001106991 |
| ENSG00000198624 | CCDC69 | −0.71903 | 5.93E−07 | 1.57E−05 |
| ENSG00000103888 | CEMIP | −0.71896 | 8.27E−10 | 4.19E−08 |
| ENSG00000187140 | FOXD3 | −0.71852 | 1.10E−09 | 5.36E−08 |
| ENSG00000198964 | SGMS1 | −0.71815 | 3.26E−15 | 4.21E−13 |
| ENSG00000224081 | SLC44A3-AS1 | −0.71779 | 0.002919 | 0.022799652 |
| ENSG00000173991 | TCAP | −0.71738 | 0.007462 | 0.047053227 |
| ENSG00000151834 | GABRA2 | −0.7168 | 0.000267 | 0.003197598 |
| ENSG00000233215 | LINC01687 | −0.71598 | 0.001789 | 0.015455035 |
| ENSG00000137764 | MAP2K5 | −0.71572 | 0.00055 | 0.005858441 |
| ENSG00000158008 | EXTL1 | −0.71523 | 1.03E−09 | 5.06E−08 |
| ENSG00000114023 | FAM162A | −0.71465 | 1.35E−10 | 8.25E−09 |
| ENSG00000108387 | 44443 | −0.71336 | 0.003177 | 0.024333664 |
| ENSG00000155761 | SPAG17 | −0.71035 | 4.76E−05 | 0.000731244 |
| ENSG00000112964 | GHR | −0.70807 | 0.000598 | 0.00626024 |
| ENSG00000267801 | AC087289.5 | −0.70801 | 0.002778 | 0.021882498 |
| ENSG00000137460 | FHDC1 | −0.70722 | 1.56E−08 | 5.73E−07 |
| ENSG00000091592 | NLRP1 | −0.70708 | 9.45E−10 | 4.70E−08 |
| ENSG00000054282 | SDCCAG8 | −0.70662 | 9.67E−10 | 4.77E−08 |
| ENSG00000009694 | TENM1 | −0.70593 | 1.32E−06 | 3.24E−05 |
| ENSG00000164764 | SBSPON | −0.70505 | 9.99E−05 | 0.001376626 |
| ENSG00000172264 | MACROD2 | −0.70496 | 6.05E−05 | 0.00090404 |
| ENSG00000196502 | SULT1A1 | −0.70452 | 1.19E−10 | 7.41E−09 |
| ENSG00000261786 | AC006058.1 | −0.70442 | 0.001836 | 0.015778291 |
| ENSG00000060566 | CREB3L3 | −0.70402 | 0.00132 | 0.012050244 |
| ENSG00000020181 | ADGRA2 | −0.70286 | 5.32E−09 | 2.16E−07 |
| ENSG00000120658 | ENOX1 | −0.7019 | 1.44E−07 | 4.31E−06 |
| ENSG00000163590 | PPM1L | −0.70169 | 4.23E−05 | 0.000660319 |
| ENSG00000106526 | ACTR3C | −0.70117 | 1.29E−05 | 0.000238167 |
| ENSG00000112276 | BVES | −0.70081 | 1.19E−11 | 9.21E−10 |
| ENSG00000181449 | SOX2 | −0.70059 | 4.72E−07 | 1.28E−05 |
| ENSG00000077380 | DYNC1I2 | −0.6997 | 1.23E−12 | 1.13E−10 |
| ENSG00000171033 | PKIA | −0.69849 | 3.29E−05 | 0.000535838 |
| ENSG00000213626 | LBH | −0.69724 | 4.89E−07 | 1.32E−05 |
| ENSG00000235109 | ZSCAN31 | −0.69719 | 1.52E−13 | 1.58E−11 |
| ENSG00000203727 | SAMD5 | −0.69693 | 6.80E−06 | 0.000135647 |
| ENSG00000070731 | ST6GALNAC2 | −0.69627 | 1.02E−08 | 3.91E−07 |
| ENSG00000223865 | HLA-DPB1 | −0.69601 | 3.02E−09 | 1.31E−07 |
| ENSG00000143995 | MEIS1 | −0.69546 | 6.92E−09 | 2.75E−07 |
| ENSG00000120458 | MSANTD2 | −0.69544 | 5.14E−07 | 1.38E−05 |
| ENSG00000283632 | EXOC3L2 | −0.69468 | 0.004006 | 0.028992759 |
| ENSG00000272622 | AC010735.2 | −0.69271 | 5.43E−08 | 1.79E−06 |
| ENSG00000134121 | CHL1 | −0.69268 | 1.23E−08 | 4.66E−07 |
| ENSG00000278530 | CHMP1B2P | −0.69242 | 2.08E−05 | 0.000358064 |
| ENSG00000185760 | KCNQ5 | −0.69193 | 0.000317 | 0.003695466 |
| ENSG00000111816 | FRK | −0.69113 | 0.000306 | 0.003592837 |
| ENSG00000106804 | C5 | −0.69001 | 0.000288 | 0.003410343 |
| ENSG00000247134 | AC090204.1 | −0.68948 | 0.000131 | 0.00174026 |
| ENSG00000278535 | DHRS11 | −0.6876 | 2.23E−10 | 1.30E−08 |
| ENSG00000251129 | LINC02506 | −0.68697 | 1.39E−08 | 5.15E−07 |
| ENSG00000104936 | DMPK | −0.68571 | 3.24E−16 | 4.84E−14 |
| ENSG00000183853 | KIRREL1 | −0.68461 | 1.29E−09 | 6.12E−08 |
| ENSG00000143512 | HHIPL2 | −0.68412 | 0.002365 | 0.01928157 |
| ENSG00000259786 | LINC02109 | −0.68402 | 0.001629 | 0.014357456 |
| ENSG00000198121 | LPAR1 | −0.68304 | 0.001101 | 0.010430331 |
| ENSG00000079691 | CARMIL1 | −0.68276 | 8.45E−14 | 9.10E−12 |
| ENSG00000164796 | CSMD3 | −0.68234 | 3.13E−05 | 0.000516135 |
| ENSG00000130382 | MLLT1 | −0.68204 | 1.67E−05 | 0.000296534 |
| ENSG00000167676 | PLIN4 | −0.6819 | 1.87E−11 | 1.39E−09 |
| ENSG00000136275 | C7orf69 | −0.68127 | 0.003901 | 0.028331488 |
| ENSG00000115556 | PLCD4 | −0.68119 | 8.89E−06 | 0.00017198 |
| ENSG00000205403 | CFI | −0.68025 | 4.02E−11 | 2.74E−09 |
| ENSG00000162944 | RFTN2 | −0.68021 | 0.004063 | 0.029307272 |
| ENSG00000166575 | TMEM135 | −0.67885 | 4.93E−11 | 3.25E−09 |
| ENSG00000240583 | AQP1 | −0.67845 | 3.38E−08 | 1.15E−06 |
| ENSG00000115896 | PLCL1 | −0.6782 | 0.000209 | 0.002594612 |
| ENSG00000182752 | PAPPA | −0.6776 | 2.35E−08 | 8.38E−07 |
| ENSG00000267534 | S1PR2 | −0.67691 | 2.77E−08 | 9.72E−07 |
| ENSG00000196814 | MVB12B | −0.67671 | 1.74E−07 | 5.13E−06 |
| ENSG00000154529 | CNTNAP3B | −0.67494 | 0.001101 | 0.010430331 |
| ENSG00000166780 | C16orf45 | −0.67417 | 8.27E−10 | 4.19E−08 |
| ENSG00000099998 | GGT5 | −0.67408 | 0.001243 | 0.01148273 |
| ENSG00000126803 | HSPA2 | −0.67406 | 2.21E−15 | 2.89E−13 |
| ENSG00000169067 | ACTBL2 | −0.67307 | 0.004893 | 0.033605558 |
| ENSG00000128606 | LRRC17 | −0.67163 | 2.42E−09 | 1.07E−07 |
| ENSG00000132535 | DLG4 | −0.66974 | 4.94E−05 | 0.000755296 |
| ENSG00000164175 | SLC45A2 | −0.66974 | 1.30E−07 | 3.93E−06 |
| ENSG00000099957 | P2RX6 | −0.66857 | 4.32E−08 | 1.45E−06 |
| ENSG00000239521 | CASTOR3 | −0.66834 | 3.74E−06 | 8.12E−05 |
| ENSG00000163072 | NOSTRIN | −0.66768 | 0.000499 | 0.005400948 |
| ENSG00000116353 | MECR | −0.66759 | 2.42E−07 | 6.92E−06 |
| ENSG00000179403 | VWA1 | −0.66744 | 0.001494 | 0.013333168 |
| ENSG00000071575 | TRIB2 | −0.66665 | 9.77E−17 | 1.56E−14 |
| ENSG00000184903 | IMMP2L | −0.66618 | 3.32E−09 | 1.43E−07 |
| ENSG00000171951 | SCG2 | −0.66589 | 2.80E−10 | 1.59E−08 |
| ENSG00000144355 | DLX1 | −0.66555 | 1.21E−11 | 9.32E−10 |
| ENSG00000242574 | HLA-DMB | −0.66465 | 1.67E−08 | 6.11E−07 |
| ENSG00000164683 | HEY1 | −0.66452 | 4.39E−09 | 1.83E−07 |
| ENSG00000141052 | MYOCD | −0.66394 | 8.35E−07 | 2.14E−05 |
| ENSG00000163637 | PRICKLE2 | −0.66391 | 1.67E−09 | 7.69E−08 |
| ENSG00000125730 | C3 | −0.66348 | 8.01E−12 | 6.33E−10 |
| ENSG00000198756 | COLGALT2 | −0.66175 | 1.98E−05 | 0.000342235 |
| ENSG00000261379 | AC010735.1 | −0.66175 | 6.56E−06 | 0.000131595 |
| ENSG00000048740 | CELF2 | −0.66116 | 0.000582 | 0.006144055 |
| ENSG00000154237 | LRRK1 | −0.66112 | 4.48E−06 | 9.51E−05 |
| ENSG00000243944 | AC117386.2 | −0.66101 | 0.000358 | 0.004097982 |
| ENSG00000119943 | PYROXD2 | −0.66023 | 3.35E−06 | 7.35E−05 |
| ENSG00000169499 | PLEKHA2 | −0.66013 | 4.74E−16 | 6.96E−14 |
| ENSG00000185477 | GPRIN3 | −0.65996 | 1.56E−09 | 7.18E−08 |
| ENSG00000116819 | TFAP2E | −0.65975 | 0.003875 | 0.028239454 |
| ENSG00000135842 | FAM129A | −0.6591 | 1.53E−12 | 1.40E−10 |
| ENSG00000162496 | DHRS3 | −0.65855 | 1.79E−12 | 1.60E−10 |
| ENSG00000121858 | TNFSF10 | −0.65617 | 3.78E−07 | 1.05E−05 |
| ENSG00000139044 | B4GALNT3 | −0.65575 | 2.25E−10 | 1.31E−08 |
| ENSG00000180447 | GAS1 | −0.65423 | 1.55E−06 | 3.74E−05 |
| ENSG00000187678 | SPRY4 | −0.65366 | 1.65E−05 | 0.000292853 |
| ENSG00000223564 | CYP4F32P | −0.65273 | 5.24E−06 | 0.000109167 |
| ENSG00000270885 | RASL10B | −0.65264 | 7.19E−06 | 0.00014273 |
| ENSG00000120725 | SIL1 | −0.65207 | 1.60E−11 | 1.20E−09 |
| ENSG00000179583 | CIITA | −0.65078 | 1.48E−05 | 0.000268069 |
| ENSG00000250337 | PURPL | −0.64941 | 2.58E−08 | 9.12E−07 |
| ENSG00000011105 | TSPAN9 | −0.64837 | 9.21E−08 | 2.89E−06 |
| ENSG00000044524 | EPHA3 | −0.64701 | 1.08E−09 | 5.25E−08 |
| ENSG00000175899 | A2M | −0.6461 | 2.71E−10 | 1.55E−08 |
| ENSG00000188177 | ZC3H6 | −0.64485 | 1.34E−05 | 0.000246164 |
| ENSG00000165675 | ENOX2 | −0.64482 | 1.65E−10 | 9.86E−09 |
| ENSG00000159733 | ZFYVE28 | −0.6436 | 6.10E−05 | 0.000910061 |
| ENSG00000175745 | NR2F1 | −0.64333 | 2.89E−05 | 0.000480441 |
| ENSG00000170647 | NA | −0.6428 | 0.004492 | 0.03148652 |
| ENSG00000173930 | SLCO4C1 | −0.64255 | 0.005142 | 0.034893425 |
| ENSG00000154122 | ANKH | −0.64143 | 2.72E−10 | 1.55E−08 |
| ENSG00000104490 | NCALD | −0.64115 | 7.78E−08 | 2.49E−06 |
| ENSG00000204287 | HLA-DRA | −0.64104 | 7.49E−11 | 4.85E−09 |
| ENSG00000119699 | TGFB3 | −0.64078 | 2.28E−08 | 8.16E−07 |
| ENSG00000139173 | TMEM117 | −0.64051 | 0.000157 | 0.002015249 |
| ENSG00000117069 | ST6GALNAC5 | −0.63973 | 0.000296 | 0.003487428 |
| ENSG00000106546 | AHR | −0.63906 | 5.74E−13 | 5.58E−11 |
| ENSG00000069702 | TGFBR3 | −0.63903 | 6.24E−09 | 2.50E−07 |
| ENSG00000116661 | FBXO2 | −0.63803 | 0.000421 | 0.00467316 |
| ENSG00000137868 | STRA6 | −0.63693 | 2.00E−09 | 8.98E−08 |
| ENSG00000189186 | DCAF8L2 | −0.63655 | 1.91E−05 | 0.000331576 |
| ENSG00000100299 | ARSA | −0.63633 | 2.83E−05 | 0.000471913 |
| ENSG00000203857 | HSD3B1 | −0.63562 | 7.79E−05 | 0.001119915 |
| ENSG00000267284 | AC022031.2 | −0.63526 | 0.001927 | 0.016380173 |
| ENSG00000175874 | CREG2 | −0.63515 | 0.000587 | 0.006175966 |
| ENSG00000198908 | BHLHB9 | −0.63474 | 0.000944 | 0.009200419 |
| ENSG00000120156 | TEK | −0.63469 | 6.49E−08 | 2.10E−06 |
| ENSG00000106780 | MEGF9 | −0.63402 | 4.05E−12 | 3.46E−10 |
| ENSG00000196739 | COL27A1 | −0.63353 | 1.32E−06 | 3.25E−05 |
| ENSG00000234323 | LINC01505 | −0.63288 | 8.69E−06 | 0.000168554 |
| ENSG00000135362 | PRR5L | −0.63276 | 4.68E−10 | 2.50E−08 |
| ENSG00000206567 | AC022007.1 | −0.63271 | 0.000514 | 0.005536832 |
| ENSG00000133055 | MYBPH | −0.63218 | 7.37E−05 | 0.001068578 |
| ENSG00000257354 | AC048341.1 | −0.63207 | 0.003531 | 0.026291251 |
| ENSG00000157388 | CACNA1D | −0.63108 | 0.002233 | 0.018453963 |
| ENSG00000115461 | IGFBP5 | −0.63102 | 0.000343 | 0.0039529 |
| ENSG00000248429 | FAM198B-AS1 | −0.63072 | 0.004549 | 0.03183922 |
| ENSG00000198909 | MAP3K3 | −0.62996 | 1.31E−09 | 6.21E−08 |
| ENSG00000188153 | COL4A5 | −0.62928 | 2.08E−09 | 9.29E−08 |
| ENSG00000100321 | SYNGR1 | −0.62884 | 5.69E−10 | 2.96E−08 |
| ENSG00000249395 | CASC9 | −0.62788 | 1.21E−06 | 2.99E−05 |
| ENSG00000010810 | FYN | −0.62729 | 3.99E−11 | 2.73E−09 |
| ENSG00000073417 | PDE8A | −0.62629 | 8.28E−10 | 4.19E−08 |
| ENSG00000143248 | RGS5 | −0.62617 | 1.57E−08 | 5.76E−07 |
| ENSG00000141441 | GAREM1 | −0.62611 | 5.71E−08 | 1.87E−06 |
| ENSG00000154493 | C10orf90 | −0.62597 | 3.51E−05 | 0.000567379 |
| ENSG00000258932 | AL390334.1 | −0.62593 | 0.005853 | 0.038867956 |
| ENSG00000135929 | CYP27A1 | −0.62502 | 9.82E−11 | 6.22E−09 |
| ENSG00000172915 | NBEA | −0.62488 | 3.15E−09 | 1.36E−07 |
| ENSG00000103591 | AAGAB | −0.62476 | 4.07E−12 | 3.46E−10 |
| ENSG00000138080 | EMILIN1 | −0.62465 | 2.43E−10 | 1.41E−08 |
| ENSG00000188015 | S100A3 | −0.62455 | 2.19E−05 | 0.000373847 |
| ENSG00000115318 | LOXL3 | −0.62388 | 1.91E−14 | 2.19E−12 |
| ENSG00000167566 | NCKAP5L | −0.62335 | 4.28E−06 | 9.13E−05 |
| ENSG00000158352 | SHROOM4 | −0.62283 | 7.83E−06 | 0.000154026 |
| ENSG00000184557 | SOCS3 | −0.62055 | 1.29E−11 | 9.90E−10 |
| ENSG00000165949 | IFI27 | −0.61943 | 0.000277 | 0.003308989 |
| ENSG00000104368 | PLAT | −0.61936 | 3.49E−11 | 2.40E−09 |
| ENSG00000064687 | ABCA7 | −0.61847 | 2.84E−10 | 1.61E−08 |
| ENSG00000250479 | CHCHD10 | −0.61531 | 1.31E−08 | 4.88E−07 |
| ENSG00000187720 | THSD4 | −0.61395 | 3.19E−05 | 0.000522234 |
| ENSG00000167964 | RAB26 | −0.61288 | 1.71E−10 | 1.02E−08 |
| ENSG00000149294 | NCAM1 | −0.61259 | 4.99E−07 | 1.34E−05 |
| ENSG00000175662 | TOM1L2 | −0.61258 | 3.67E−05 | 0.000586554 |
| ENSG00000132688 | NES | −0.61185 | 3.91E−10 | 2.14E−08 |
| ENSG00000120659 | TNFSF11 | −0.61113 | 9.69E−07 | 2.44E−05 |
| ENSG00000018236 | CNTN1 | −0.61031 | 0.00048 | 0.005249173 |
| ENSG00000003249 | DBNDD1 | −0.60983 | 1.91E−05 | 0.000332442 |
| ENSG00000184384 | MAML2 | −0.60851 | 6.79E−05 | 0.001000305 |
| ENSG00000105711 | SCN1B | −0.60802 | 1.82E−06 | 4.31E−05 |
| ENSG00000087884 | AAMDC | −0.60766 | 0.000613 | 0.006384028 |
| ENSG00000235501 | AC105942.1 | −0.6072 | 0.00709 | 0.045108485 |
| ENSG00000113248 | PCDHB15 | −0.60689 | 0.000102 | 0.001403793 |
| ENSG00000144642 | RBMS3 | −0.60608 | 1.15E−09 | 5.59E−08 |
| ENSG00000136158 | SPRY2 | −0.6059 | 1.53E−10 | 9.16E−09 |
| ENSG00000283117 | AC004949.1 | −0.60558 | 0.00013 | 0.00172902 |
| ENSG00000239589 | LINC00879 | −0.60515 | 0.003655 | 0.027019627 |
| ENSG00000132530 | XAF1 | −0.60481 | 0.007821 | 0.048824978 |
| ENSG00000185585 | OLFML2A | −0.60369 | 0.00015 | 0.001943693 |
| ENSG00000108515 | ENO3 | −0.60257 | 8.22E−07 | 2.11E−05 |
| ENSG00000186469 | GNG2 | −0.60027 | 3.66E−10 | 2.03E−08 |
| ENSG00000130203 | APOE | −0.60007 | 2.64E−11 | 1.88E−09 |
| ENSG00000225285 | LINC01770 | −0.5999 | 0.006666 | 0.043065924 |
| ENSG00000144847 | IGSF11 | −0.59941 | 0.00387 | 0.028212044 |
| ENSG00000142687 | KIAA0319L | −0.59917 | 6.62E−13 | 6.34E−11 |
| ENSG00000177409 | SAMD9L | −0.59785 | 6.66E−12 | 5.37E−10 |
| ENSG00000247081 | BAALC-AS1 | −0.5946 | 0.00057 | 0.006042526 |
| ENSG00000116667 | C1orf21 | −0.59452 | 7.40E−06 | 0.000146646 |
| ENSG00000165948 | IFI27L1 | −0.59447 | 8.10E−05 | 0.001160039 |
| ENSG00000250510 | GPR162 | −0.59346 | 4.84E−05 | 0.000741638 |
| ENSG00000049130 | KITLG | −0.59246 | 1.73E−06 | 4.11E−05 |
| ENSG00000068024 | HDAC4 | −0.59228 | 2.65E−07 | 7.53E−06 |
| ENSG00000120833 | SOCS2 | −0.5921 | 0.000113 | 0.001526906 |
| ENSG00000156869 | FRRS1 | −0.59061 | 6.15E−08 | 1.99E−06 |
| ENSG00000178764 | ZHX2 | −0.58907 | 0.000999 | 0.009643571 |
| ENSG00000089041 | P2RX7 | −0.58887 | 0.000524 | 0.005616314 |
| ENSG00000130164 | LDLR | −0.58801 | 4.01E−12 | 3.44E−10 |
| ENSG00000171004 | HS6ST2 | −0.588 | 3.77E−06 | 8.17E−05 |
| ENSG00000090565 | RAB11FIP3 | −0.58693 | 2.96E−09 | 1.29E−07 |
| ENSG00000007237 | GAS7 | −0.58639 | 3.12E−08 | 1.07E−06 |
| ENSG00000113594 | LIFR | −0.58622 | 1.36E−12 | 1.25E−10 |
| ENSG00000164761 | TNFRSF11B | −0.58448 | 1.39E−05 | 0.000253866 |
| ENSG00000011677 | GABRA3 | −0.58407 | 0.003427 | 0.025758004 |
| ENSG00000006210 | CX3CL1 | −0.58349 | 0.001931 | 0.016394424 |
| ENSG00000112146 | FBXO9 | −0.58254 | 1.84E−09 | 8.37E−08 |
| ENSG00000134013 | LOXL2 | −0.58243 | 4.17E−11 | 2.80E−09 |
| ENSG00000204257 | HLA-DMA | −0.58204 | 4.91E−06 | 0.000103226 |
| ENSG00000113578 | FGF1 | −0.58168 | 0.000723 | 0.007310412 |
| ENSG00000237187 | NR2F1-AS1 | −0.58138 | 0.003896 | 0.028331488 |

List of genes that are up-regulated upon TRASH-ASO treatment.

| | | | | |
|---|---|---|---|---|
| ENSG00000110244 | APOA4 | 7.546921 | 4.37E−10 | 2.35E−08 |
| ENSG00000163295 | ALPI | 7.152811 | 1.24E−08 | 4.68E−07 |
| ENSG00000171487 | NLRP5 | 6.996995 | 1.00E−08 | 3.83E−07 |
| ENSG00000130294 | KIF1A | 6.60377 | 8.87E−08 | 2.80E−06 |
| ENSG00000179148 | ALOXE3 | 4.940284 | 1.07E−05 | 0.000200717 |
| ENSG00000135750 | KCNK1 | 4.667929 | 5.17E−07 | 1.38E−05 |
| ENSG00000120279 | MYCT1 | 3.863058 | 7.46E−07 | 1.93E−05 |
| ENSG00000153531 | ADPRHL1 | 3.705736 | 1.55E−83 | 1.47E−79 |
| ENSG00000100368 | CSF2RB | 3.672316 | 5.20E−06 | 0.000108627 |
| ENSG00000183778 | B3GALT5 | 3.379573 | 4.02E−18 | 7.85E−16 |
| ENSG00000126217 | MCF2L | 3.366648 | 9.17E−07 | 2.32E−05 |
| ENSG00000131737 | KRT34 | 3.320354 | 4.48E−08 | 1.50E−06 |
| ENSG00000164266 | SPINK1 | 3.070383 | 1.78E−31 | 2.25E−28 |
| ENSG00000101203 | COL20A1 | 2.951345 | 3.62E−06 | 7.89E−05 |
| ENSG00000257495 | KRT73-AS1 | 2.887516 | 3.11E−07 | 8.75E−06 |
| ENSG00000260220 | CCDC187 | 2.880767 | 8.57E−06 | 0.000166792 |
| ENSG00000078549 | ADCYAP1R1 | 2.870417 | 3.08E−11 | 2.17E−09 |
| ENSG00000155093 | PTPRN2 | 2.844389 | 9.16E−07 | 2.32E−05 |
| ENSG00000026751 | SLAMF7 | 2.821302 | 3.51E−40 | 1.11E−36 |
| ENSG00000145113 | MUC4 | 2.705729 | 1.79E−24 | 9.17E−22 |
| ENSG00000154133 | ROBO4 | 2.692127 | 3.05E−08 | 1.05E−06 |
| ENSG00000157087 | ATP2B2 | 2.6809 | 1.89E−05 | 0.000329257 |
| ENSG00000283265 | AL356234.3 | 2.667874 | 8.17E−11 | 5.27E−09 |
| ENSG00000114204 | SERPINI2 | 2.644947 | 1.40E−05 | 0.00025487 |
| ENSG00000047617 | ANO2 | 2.642934 | 1.26E−14 | 1.47E−12 |
| ENSG00000217825 | AC099552.1 | 2.619098 | 4.85E−09 | 2.00E−07 |
| ENSG00000283646 | LINC02009 | 2.6101 | 4.45E−19 | 9.69E−17 |
| ENSG00000142623 | PADI1 | 2.566464 | 3.95E−05 | 0.000623371 |
| ENSG00000162711 | NLRP3 | 2.515037 | 8.90E−08 | 2.81E−06 |
| ENSG00000162723 | SLAMF9 | 2.487104 | 1.88E−06 | 4.45E−05 |
| ENSG00000167850 | CD300C | 2.474447 | 4.43E−15 | 5.60E−13 |
| ENSG00000162892 | IL24 | 2.41222 | ######## | 1.32E−124 |
| ENSG00000258791 | LINC00520 | 2.380053 | 2.00E−29 | 2.11E−26 |
| ENSG00000123977 | DAW1 | 2.337999 | 4.07E−05 | 0.000638929 |
| ENSG00000123569 | H2BFWT | 2.335671 | 3.84E−05 | 0.000609832 |
| ENSG00000111305 | GSG1 | 2.331529 | 4.29E−10 | 2.32E−08 |
| ENSG00000222047 | C10orf55 | 2.330936 | 0.000209 | 0.002592579 |
| ENSG00000167751 | KLK2 | 2.320597 | 0.000146 | 0.001892105 |
| ENSG00000007314 | SCN4A | 2.300691 | 1.19E−09 | 5.75E−08 |
| ENSG00000175841 | FAM172BP | 2.272948 | 1.67E−06 | 3.99E−05 |
| ENSG00000124343 | XG | 2.255526 | 9.00E−06 | 0.000173821 |
| ENSG00000167642 | SPINT2 | 2.192074 | 5.45E−07 | 1.45E−05 |
| ENSG00000125740 | FOSB | 2.191016 | 1.39E−23 | 5.99E−21 |
| ENSG00000197046 | SIGLEC15 | 2.101248 | 0.000724 | 0.007323124 |
| ENSG00000144583 | 44259 | 2.095246 | 3.40E−19 | 7.57E−17 |
| ENSG00000179046 | TRIML2 | 2.081298 | 1.81E−05 | 0.000316519 |
| ENSG00000155961 | RAB39B | 2.060244 | 4.29E−12 | 3.62E−10 |
| ENSG00000189320 | FAM180A | 2.046828 | 0.000416 | 0.004621126 |
| ENSG00000133083 | DCLK1 | 2.000771 | 1.53E−15 | 2.09E−13 |
| ENSG00000248964 | AC131254.1 | 2.000124 | 0.000808 | 0.008060374 |
| ENSG00000238266 | LINC00707 | 1.936375 | 2.87E−13 | 2.89E−11 |
| ENSG00000164746 | C7orf57 | 1.917137 | 2.53E−08 | 8.99E−07 |
| ENSG00000170454 | KRT75 | 1.914512 | 1.88E−09 | 8.52E−08 |
| ENSG00000106018 | VIPR2 | 1.909907 | 0.000342 | 0.003945836 |
| ENSG00000240891 | PLCXD2 | 1.881581 | 2.53E−68 | 1.60E−64 |
| ENSG00000260604 | AL590004.4 | 1.853096 | 7.15E−32 | 9.68E−29 |
| ENSG00000231802 | AC009502.2 | 1.847117 | 0.000407 | 0.004537175 |
| ENSG00000172137 | CALB2 | 1.839132 | 5.41E−12 | 4.42E−10 |
| ENSG00000230439 | AL512488.1 | 1.823599 | 1.50E−10 | 9.04E−09 |
| ENSG00000255145 | STX17-AS1 | 1.8172 | 0.000595 | 0.006237548 |
| ENSG00000149968 | MMP3 | 1.814558 | 1.26E−12 | 1.16E−10 |
| ENSG00000115423 | DNAH6 | 1.797947 | 4.83E−11 | 3.20E−09 |
| ENSG00000197181 | PIWIL2 | 1.786524 | 5.23E−09 | 2.14E−07 |
| ENSG00000184515 | BEX5 | 1.77838 | 5.02E−12 | 4.14E−10 |
| ENSG00000105877 | DNAH11 | 1.74955 | 1.33E−09 | 6.28E−08 |
| ENSG00000240602 | AADACP1 | 1.74415 | 9.52E−10 | 4.72E−08 |
| ENSG00000243742 | RPLPOP2 | 1.743845 | 1.92E−17 | 3.40E−15 |
| ENSG00000175920 | DOK7 | 1.741274 | 0.00032 | 0.00372572 |
| ENSG00000196557 | CACNA1H | 1.732619 | 0.002355 | 0.019229793 |
| ENSG00000187800 | PEAR1 | 1.726431 | 6.31E−09 | 2.52E−07 |
| ENSG00000167984 | NLRC3 | 1.712694 | 2.52E−05 | 0.000424895 |
| ENSG00000142619 | PADI3 | 1.708203 | 4.16E−11 | 2.80E−09 |
| ENSG00000170498 | KISS1 | 1.678698 | 7.99E−08 | 2.54E−06 |
| ENSG00000181652 | ATG9B | 1.674441 | 2.72E−14 | 3.04E−12 |
| ENSG00000165105 | RASEF | 1.673873 | 1.43E−26 | 8.45E−24 |
| ENSG00000179242 | CDH4 | 1.66833 | 0.007678 | 0.048090506 |
| ENSG00000260653 | AC237221.1 | 1.664886 | 0.003741 | 0.027469645 |
| ENSG00000106952 | TNFSF8 | 1.660308 | 3.52E−06 | 7.69E−05 |
| ENSG00000234805 | AC090505.1 | 1.656292 | 4.56E−05 | 0.000706399 |
| ENSG00000148346 | LCN2 | 1.646988 | 0.001069 | 0.010189194 |
| ENSG00000160255 | ITGB2 | 1.646373 | 0.001655 | 0.014517829 |
| ENSG00000167992 | VWCE | 1.638594 | 9.59E−06 | 0.000183099 |
| ENSG00000275216 | AL161431.1 | 1.630785 | 2.06E−09 | 9.20E−08 |
| ENSG00000233221 | AC133785.1 | 1.627398 | 0.000576 | 0.006091198 |
| ENSG00000166816 | LDHD | 1.622896 | 1.99E−06 | 4.66E−05 |
| ENSG00000166922 | SCG5 | 1.620959 | 9.17E−35 | 1.58E−31 |
| ENSG00000189001 | SBSN | 1.620691 | 0.000204 | 0.002530217 |
| ENSG00000139973 | SYT16 | 1.619882 | 0.00096 | 0.009316604 |
| ENSG00000171346 | KRT15 | 1.605004 | 1.20E−13 | 1.27E−11 |
| ENSG00000163833 | FBXO40 | 1.588739 | 0.007784 | 0.048628062 |
| ENSG00000182261 | NLRP10 | 1.584479 | 0.000139 | 0.001819599 |
| ENSG00000283517 | AC005144.1 | 1.57828 | 0.000379 | 0.004273816 |
| ENSG00000236719 | OVAAL | 1.572278 | 1.38E−16 | 2.14E−14 |
| ENSG00000256982 | AC135782.1 | 1.56338 | 3.63E−07 | 1.01E−05 |
| ENSG00000253227 | AC090192.2 | 1.560917 | 5.02E−06 | 0.000105148 |
| ENSG00000186642 | PDE2A | 1.552764 | 7.56E−17 | 1.22E−14 |
| ENSG00000233521 | LINC01638 | 1.543752 | 1.76E−08 | 6.40E−07 |
| ENSG00000229563 | LINC01204 | 1.54317 | 3.91E−05 | 0.000618927 |
| ENSG00000101197 | BIRC7 | 1.539998 | 4.77E−06 | 0.000100619 |
| ENSG00000156265 | MAP3K7CL | 1.530035 | 4.29E−05 | 0.000668241 |
| ENSG00000240244 | GAPDHP33 | 1.51435 | 0.00015 | 0.001947232 |
| ENSG00000002079 | MYH16 | 1.514187 | 0.000472 | 0.005177689 |
| ENSG00000126860 | EVI2A | 1.514132 | 0.004597 | 0.032023549 |
| ENSG00000265190 | ANXA8 | 1.512834 | 9.27E−09 | 3.58E−07 |
| ENSG00000184368 | MAP7D2 | 1.512689 | 0.000293 | 0.003458496 |
| ENSG00000235314 | LINC00957 | 1.503338 | 2.72E−11 | 1.93E−09 |
| ENSG00000111348 | ARHGDIB | 1.500735 | 6.07E−10 | 3.14E−08 |
| ENSG00000198574 | SH2D1B | 1.494503 | 3.85E−07 | 1.06E−05 |
| ENSG00000175746 | C15orf54 | 1.488861 | 4.73E−10 | 2.52E−08 |
| ENSG00000177699 | AC011944.1 | 1.481254 | 0.004656 | 0.032374824 |
| ENSG00000082126 | MPP4 | 1.479352 | 1.19E−14 | 1.40E−12 |
| ENSG00000264301 | LINC01444 | 1.478755 | 0.000436 | 0.004811326 |
| ENSG00000237870 | AC073130.1 | 1.474273 | 5.49E−06 | 0.000113397 |
| ENSG00000205683 | DPF3 | 1.472931 | 0.000416 | 0.004623826 |
| ENSG00000181790 | ADGRB1 | 1.472108 | 1.06E−05 | 0.000199354 |
| ENSG00000205038 | PKHD1L1 | 1.460494 | 0.003104 | 0.023947885 |
| ENSG00000153233 | PTPRR | 1.4591 | 2.03E−18 | 4.22E−16 |
| ENSG00000268758 | ADGRE4P | 1.451307 | 0.001653 | 0.014509777 |
| ENSG00000277778 | PGM5P2 | 1.446654 | 4.32E−14 | 4.76E−12 |
| ENSG00000186047 | DLEU7 | 1.438271 | 0.000131 | 0.001740879 |
| ENSG00000231131 | LINC01468 | 1.432513 | 9.08E−09 | 3.51E−07 |
| ENSG00000258998 | LINC02302 | 1.423816 | 0.003279 | 0.024952707 |
| ENSG00000272068 | AL365181.2 | 1.421754 | 0.000174 | 0.002197961 |
| ENSG00000166396 | SERPINB7 | 1.414751 | 6.75E−38 | 1.60E−34 |
| ENSG00000165215 | CLDN3 | 1.401619 | 7.07E−06 | 0.000140416 |
| ENSG00000181126 | HLA-V | 1.397637 | 6.58E−08 | 2.12E−06 |
| ENSG00000163827 | LRRC2 | 1.393556 | 0.000395 | 0.004430167 |
| ENSG00000169908 | TM4SF1 | 1.389468 | 3.72E−48 | 1.41E−44 |
| ENSG00000113555 | PCDH12 | 1.386405 | 4.14E−08 | 1.40E−06 |
| ENSG00000182795 | C1orf116 | 1.381528 | 1.37E−09 | 6.45E−08 |
| ENSG00000185567 | AHNAK2 | 1.378785 | 4.14E−25 | 2.31E−22 |
| ENSG00000179817 | MRGPRX4 | 1.36715 | 0.002619 | 0.020902531 |
| ENSG00000147394 | ZNF185 | 1.367112 | 1.01E−10 | 6.31E−09 |
| ENSG00000117152 | RGS4 | 1.365747 | 1.53E−08 | 5.63E−07 |
| ENSG00000169252 | ADRB2 | 1.354156 | 1.68E−19 | 3.98E−17 |
| ENSG00000259518 | LINC01583 | 1.348012 | 2.22E−05 | 0.000379057 |
| ENSG00000186310 | NAP1L3 | 1.339387 | 1.73E−08 | 6.33E−07 |
| ENSG00000240476 | LINC00973 | 1.338991 | 2.52E−24 | 1.23E−21 |
| ENSG00000258590 | NBEAP1 | 1.338334 | 0.00022 | 0.002712453 |
| ENSG00000198573 | SPANXC | 1.330886 | 0.001847 | 0.015845118 |
| ENSG00000132718 | SYT11 | 1.325537 | 8.29E−53 | 3.93E−49 |
| ENSG00000167861 | HID1 | 1.325389 | 0.006454 | 0.041996029 |
| ENSG00000248596 | AC139491.2 | 1.32206 | 4.35E−05 | 0.000676526 |
| ENSG00000188818 | ZDHHC11 | 1.322053 | 0.006549 | 0.042499009 |
| ENSG00000165606 | DRGX | 1.312109 | 0.00035 | 0.004028713 |
| ENSG00000146070 | PLA2G7 | 1.311803 | 3.87E−05 | 0.000613079 |
| ENSG00000182866 | LCK | 1.307491 | 0.00347 | 0.025955325 |
| ENSG00000119547 | ONECUT2 | 1.305774 | 0.005584 | 0.037334439 |
| ENSG00000269927 | AC004817.3 | 1.30341 | 3.35E−07 | 9.38E−06 |
| ENSG00000108309 | RUNDC3A | 1.301551 | 1.39E−11 | 1.06E−09 |
| ENSG00000264230 | ANXA8L1 | 1.298886 | 3.38E−10 | 1.90E−08 |
| ENSG00000162510 | MATN1 | 1.294304 | 0.000331 | 0.00383244 |
| ENSG00000231419 | LINC00689 | 1.29001 | 7.12E−12 | 5.67E−10 |
| ENSG00000142910 | TINAGL1 | 1.28676 | 6.71E−07 | 1.76E−05 |
| ENSG00000157557 | ETS2 | 1.278725 | 4.00E−15 | 5.09E−13 |
| ENSG00000254842 | LINC02551 | 1.274131 | 5.65E−05 | 0.000849911 |
| ENSG00000162896 | PIGR | 1.273727 | 7.73E−07 | 1.99E−05 |
| ENSG00000186205 | 44256 | 1.273501 | 3.47E−11 | 2.40E−09 |
| ENSG00000273760 | AC245041.1 | 1.260656 | 5.51E−15 | 6.78E−13 |
| ENSG00000099338 | CATSPERG | 1.259445 | 8.68E−06 | 0.000168549 |
| ENSG00000167895 | TMC8 | 1.258801 | 3.22E−06 | 7.13E−05 |
| ENSG00000198576 | ARC | 1.256936 | 3.38E−17 | 5.82E−15 |
| ENSG00000228624 | HDAC2-AS2 | 1.256169 | 0.000228 | 0.002798123 |
| ENSG00000230002 | ALMS1-IT1 | 1.246993 | 3.55E−08 | 1.21E−06 |
| ENSG00000137709 | POU2F3 | 1.243906 | 0.00054 | 0.005759434 |
| ENSG00000187994 | RINL | 1.242299 | 1.34E−10 | 8.20E−09 |
| ENSG00000167083 | GNGT2 | 1.241492 | 1.81E−06 | 4.31E−05 |
| ENSG00000162641 | AKNAD1 | 1.238465 | 0.00306 | 0.023704106 |
| ENSG00000173237 | C11orf86 | 1.23343 | 0.00011 | 0.001494048 |
| ENSG00000197279 | ZNF165 | 1.226168 | 2.70E−08 | 9.49E−07 |
| ENSG00000120217 | CD274 | 1.225602 | 1.78E−15 | 2.38E−13 |
| ENSG00000082556 | OPRK1 | 1.219692 | 0.001855 | 0.015891533 |
| ENSG00000141469 | SLC14A1 | 1.216879 | 1.85E−27 | 1.25E−24 |
| ENSG00000050438 | SLC4A8 | 1.214911 | 3.00E−08 | 1.04E−06 |
| ENSG00000102003 | SYP | 1.211392 | 1.49E−08 | 5.51E−07 |
| ENSG00000143217 | NECTIN4 | 1.206346 | 2.66E−06 | 6.00E−05 |
| ENSG00000112195 | TREML2 | 1.204893 | 0.007046 | 0.04492868 |
| ENSG00000124249 | KCNK15 | 1.202639 | 5.04E−05 | 0.000767836 |
| ENSG00000101187 | SLCO4A1 | 1.20163 | 0.000937 | 0.009154046 |
| ENSG00000267107 | PCAT19 | 1.193064 | 1.70E−05 | 0.000300298 |
| ENSG00000197646 | PDCD1LG2 | 1.191989 | 5.36E−09 | 2.18E−07 |
| ENSG00000236969 | GGT8P | 1.181029 | 0.000696 | 0.007080786 |
| ENSG00000166111 | SVOP | 1.177521 | 0.004191 | 0.029964216 |
| ENSG00000158023 | WDR66 | 1.173815 | 9.14E−11 | 5.87E−09 |
| ENSG00000149418 | ST14 | 1.17029 | 0.004085 | 0.029427236 |
| ENSG00000140519 | RHCG | 1.166277 | 0.00063 | 0.006524451 |
| ENSG00000172548 | NIPAL4 | 1.165613 | 0.002032 | 0.01706562 |
| ENSG00000095203 | EPB41L4B | 1.164826 | 1.38E−14 | 1.60E−12 |
| ENSG00000144821 | MYH15 | 1.162778 | 9.90E−15 | 1.18E−12 |
| ENSG00000234498 | RPL13AP20 | 1.162444 | 1.08E−05 | 0.000202908 |
| ENSG00000121797 | CCRL2 | 1.161645 | 1.37E−08 | 5.11E−07 |
| ENSG00000236345 | AL354719.2 | 1.160195 | 4.85E−05 | 0.000743471 |
| ENSG00000170476 | MZB1 | 1.15844 | 4.32E−05 | 0.00067295 |
| ENSG00000134160 | TRPM1 | 1.155214 | 0.000155 | 0.00199684 |
| ENSG00000058335 | RASGRF1 | 1.152233 | 5.57E−06 | 0.000114667 |
| ENSG00000251127 | AC091173.1 | 1.14897 | 8.30E−05 | 0.001181264 |
| ENSG00000164520 | RAET1E | 1.147446 | 2.55E−12 | 2.24E−10 |
| ENSG00000163623 | NKX6-1 | 1.147286 | 2.49E−06 | 5.69E−05 |
| ENSG00000127325 | BEST3 | 1.140422 | 0.006665 | 0.043065924 |
| ENSG00000129990 | SYT5 | 1.139222 | 0.003184 | 0.024373986 |
| ENSG00000125144 | MT1G | 1.135881 | 0.00029 | 0.003429425 |
| ENSG00000188910 | GJB3 | 1.13587 | 1.87E−07 | 5.48E−06 |
| ENSG00000163395 | IGFN1 | 1.134446 | 8.18E−05 | 0.00116892 |
| ENSG00000117148 | ACTL8 | 1.132791 | 0.003583 | 0.026558889 |
| ENSG00000197106 | SLC6A17 | 1.129004 | 1.37E−08 | 5.11E−07 |
| ENSG00000129170 | CSRP3 | 1.121505 | 0.000132 | 0.00174516 |
| ENSG00000122861 | PLAU | 1.118466 | 2.73E−21 | 8.91E−19 |
| ENSG00000128422 | KRT17 | 1.116168 | 0.00562 | 0.037530988 |
| ENSG00000272405 | AL365181.3 | 1.111786 | 0.005796 | 0.038575314 |
| ENSG00000184792 | OSBP2 | 1.105607 | 4.22E−11 | 2.82E−09 |
| ENSG00000198223 | CSF2RA | 1.102892 | 0.002434 | 0.019725054 |
| ENSG00000221866 | PLXNA4 | 1.100709 | 0.000108 | 0.00147294 |
| ENSG00000138356 | AOX1 | 1.098731 | 1.27E−08 | 4.75E−07 |
| ENSG00000120129 | DUSP1 | 1.094109 | 3.70E−21 | 1.19E−18 |
| ENSG00000180914 | OXTR | 1.093188 | 8.98E−08 | 2.82E−06 |
| ENSG00000189280 | GJB5 | 1.088229 | 0.001582 | 0.014011667 |
| ENSG00000238062 | SPATA3-AS1 | 1.081523 | 0.001147 | 0.010789159 |
| ENSG00000189410 | SH2D5 | 1.079972 | 3.81E−10 | 2.10E−08 |
| ENSG00000130477 | UNC13A | 1.071155 | 5.50E−07 | 1.46E−05 |
| ENSG00000198821 | CD247 | 1.069523 | 0.001583 | 0.014011667 |
| ENSG00000230836 | LINC01293 | 1.066065 | 6.45E−15 | 7.83E−13 |
| ENSG00000269896 | AL513477.1 | 1.065809 | 6.73E−05 | 0.000994188 |
| ENSG00000095752 | IL11 | 1.063873 | 1.73E−11 | 1.29E−09 |
| ENSG00000237596 | AL138828.1 | 1.060273 | 0.000315 | 0.003678516 |
| ENSG00000276850 | AC245041.2 | 1.060179 | 3.56E−16 | 5.27E−14 |
| ENSG00000125538 | IL1B | 1.058705 | 0.000682 | 0.00695638 |
| ENSG00000099812 | MISP | 1.058017 | 2.45E−06 | 5.61E−05 |
| ENSG00000234155 | LINC02535 | 1.053731 | 0.000279 | 0.003330447 |
| ENSG00000168497 | CAVIN2 | 1.052654 | 0.004593 | 0.032004895 |
| ENSG00000134242 | PTPN22 | 1.051421 | 2.12E−08 | 7.61E−07 |
| ENSG00000166923 | GREM1 | 1.047201 | 0.004131 | 0.029678915 |
| ENSG00000214274 | ANG | 1.046853 | 6.67E−05 | 0.00098752 |
| ENSG00000119508 | NR4A3 | 1.045508 | 5.46E−06 | 0.00011302 |
| ENSG00000254634 | SMG1P6 | 1.044505 | 0.001597 | 0.014106434 |
| ENSG00000114854 | TNNC1 | 1.043446 | 3.31E−06 | 7.29E−05 |
| ENSG00000173702 | MUC13 | 1.042312 | 8.02E−19 | 1.69E−16 |
| ENSG00000249846 | LINC02021 | 1.04012 | 0.00181 | 0.015600315 |
| ENSG00000267577 | AC010327.3 | 1.039932 | 0.000864 | 0.008535815 |
| ENSG00000137393 | RNF144B | 1.039622 | 5.10E−11 | 3.35E−09 |
| ENSG00000159166 | LAD1 | 1.037752 | 1.15E−07 | 3.50E−06 |
| ENSG00000167711 | SERPINF2 | 1.036015 | 0.002698 | 0.021351805 |
| ENSG00000164778 | EN2 | 1.027705 | 4.67E−12 | 3.88E−10 |
| ENSG00000136167 | LCP1 | 1.021349 | 3.21E−20 | 8.56E−18 |
| ENSG00000163376 | KBTBD8 | 1.016859 | 1.12E−14 | 1.32E−12 |
| ENSG00000124374 | PAIP2B | 1.015191 | 0.005412 | 0.036390616 |
| ENSG00000232355 | AL603650.1 | 1.014701 | 0.001744 | 0.01513957 |
| ENSG00000163254 | CRYGC | 1.013685 | 0.003691 | 0.027194946 |
| ENSG00000237624 | OXCT2P1 | 1.009908 | 0.002654 | 0.021076783 |
| ENSG00000235034 | C19orf81 | 0.994811 | 0.003556 | 0.026412996 |
| ENSG00000108798 | ABI3 | 0.991306 | 0.001942 | 0.016457274 |
| ENSG00000090382 | LYZ | 0.99009 | 5.72E−05 | 0.000859391 |
| ENSG00000271664 | AC004890.3 | 0.987895 | 0.004175 | 0.029872477 |
| ENSG00000080166 | DCT | 0.987414 | 1.16E−09 | 5.61E−08 |
| ENSG00000103154 | NECAB2 | 0.987068 | 1.29E−05 | 0.000237827 |
| ENSG00000133665 | DYDC2 | 0.987042 | 3.36E−05 | 0.000544567 |
| ENSG00000185664 | PMEL | 0.98308 | 1.66E−12 | 1.49E−10 |
| ENSG00000204792 | LINC01291 | 0.982412 | 1.87E−12 | 1.67E−10 |
| ENSG00000138675 | FGF5 | 0.978481 | 1.55E−16 | 2.39E−14 |
| ENSG00000166455 | C16orf46 | 0.97642 | 0.000235 | 0.002864359 |
| ENSG00000184860 | SDR42E1 | 0.976162 | 1.19E−05 | 0.000221307 |
| ENSG00000174567 | GOLT1A | 0.975423 | 1.67E−07 | 4.96E−06 |
| ENSG00000223949 | ROR1-AS1 | 0.973222 | 3.66E−08 | 1.24E−06 |
| ENSG00000121769 | FABP3 | 0.972382 | 7.65E−09 | 3.01E−07 |
| ENSG00000140459 | CYP11A1 | 0.969771 | 0.00392 | 0.028428011 |
| ENSG00000128564 | VGF | 0.969103 | 2.38E−19 | 5.55E−17 |
| ENSG00000175894 | TSPEAR | 0.966216 | 0.007516 | 0.047299942 |
| ENSG00000229953 | AL590666.2 | 0.963608 | 2.41E−05 | 0.000408672 |
| ENSG00000246273 | SBF2-AS1 | 0.95739 | 9.94E−08 | 3.10E−06 |
| ENSG00000145358 | DDIT4L | 0.954668 | 0.006271 | 0.041085389 |
| ENSG00000081923 | ATP8B1 | 0.952218 | 0.000203 | 0.002525896 |
| ENSG00000164744 | SUN3 | 0.951789 | 4.01E−06 | 8.63E−05 |
| ENSG00000245522 | AC026250.1 | 0.950682 | 0.000769 | 0.007726364 |
| ENSG00000163050 | COQ8A | 0.94887 | 6.76E−26 | 3.88E−23 |
| ENSG00000023445 | BIRC3 | 0.948403 | 3.13E−11 | 2.20E−09 |
| ENSG00000049759 | NEDD4L | 0.947616 | 2.28E−14 | 2.57E−12 |
| ENSG00000176723 | ZNF843 | 0.940718 | 0.001281 | 0.011796187 |
| ENSG00000135636 | DYSF | 0.939622 | 0.002314 | 0.01896068 |
| ENSG00000060762 | MPC1 | 0.939451 | 2.73E−09 | 1.19E−07 |
| ENSG00000169548 | ZNF280A | 0.937206 | 4.78E−10 | 2.54E−08 |
| ENSG00000152409 | JMY | 0.932312 | 5.32E−19 | 1.13E−16 |
| ENSG00000235884 | LINC00941 | 0.929195 | 1.73E−07 | 5.13E−06 |
| ENSG00000105383 | CD33 | 0.928968 | 1.74E−05 | 0.000306466 |
| ENSG00000280744 | LINC01173 | 0.925435 | 0.000641 | 0.006610427 |
| ENSG00000226887 | ERVMER34-1 | 0.922213 | 0.003643 | 0.026986296 |
| ENSG00000165879 | FRAT1 | 0.914606 | 0.000613 | 0.006383117 |
| ENSG00000224596 | ZMIZ1-AS1 | 0.911277 | 0.000557 | 0.005929486 |
| ENSG00000108932 | SLC16A6 | 0.909989 | 4.63E−13 | 4.55E−11 |
| ENSG00000169715 | MT1E | 0.89976 | 1.37E−17 | 2.48E−15 |
| ENSG00000143367 | TUFT1 | 0.898414 | 9.41E−20 | 2.38E−17 |
| ENSG00000270011 | ZNF559-ZNF177 | 0.896471 | 0.000562 | 0.005962624 |
| ENSG00000111344 | RASAL1 | 0.895981 | 0.000216 | 0.002662148 |
| ENSG00000100867 | DHRS2 | 0.895348 | 8.65E−10 | 4.33E−08 |
| ENSG00000134363 | FST | 0.894689 | 2.24E−05 | 0.000382419 |
| ENSG00000248375 | AC104066.1 | 0.89412 | 0.007835 | 0.048899267 |
| ENSG00000129910 | CDH15 | 0.892104 | 5.21E−10 | 2.72E−08 |
| ENSG00000283235 | AC139493.2 | 0.888217 | 0.00254 | 0.020361623 |
| ENSG00000235961 | PNMA6A | 0.886949 | 0.004438 | 0.03118516 |
| ENSG00000163803 | PLB1 | 0.886902 | 5.23E−06 | 0.00010897 |
| ENSG00000143365 | RORC | 0.882521 | 3.23E−06 | 7.14E−05 |
| ENSG00000271020 | AC112220.2 | 0.88136 | 0.001158 | 0.01087583 |
| ENSG00000100031 | GGT1 | 0.880649 | 6.47E−13 | 6.23E−11 |
| ENSG00000147174 | GCNA | 0.879667 | 0.000501 | 0.005421655 |
| ENSG00000183780 | SLC35F3 | 0.879096 | 0.006708 | 0.043243123 |
| ENSG00000260160 | AC011468.1 | 0.872073 | 0.00372 | 0.027343852 |
| ENSG00000135437 | RDH5 | 0.867611 | 1.41E−06 | 3.45E−05 |
| ENSG00000159167 | STC1 | 0.8653 | 1.52E−19 | 3.64E−17 |
| ENSG00000198857 | HSD3BP5 | 0.860568 | 0.000142 | 0.001851868 |
| ENSG00000119737 | GPR75 | 0.860387 | 0.000865 | 0.008540306 |
| ENSG00000280046 | AC104581.4 | 0.856385 | 0.001356 | 0.01232554 |
| ENSG00000100994 | PYGB | 0.855675 | 5.69E−22 | 1.96E−19 |
| ENSG00000100385 | IL2RB | 0.853667 | 0.006899 | 0.0442086 |
| ENSG00000178150 | ZNF114 | 0.850673 | 5.18E−15 | 6.42E−13 |
| ENSG00000125148 | MT2A | 0.849194 | 2.59E−19 | 5.92E−17 |
| ENSG00000167972 | ABCA3 | 0.848903 | 0.003681 | 0.027155635 |
| ENSG00000145088 | EAF2 | 0.847062 | 0.000553 | 0.005883876 |
| ENSG00000143322 | ABL2 | 0.844732 | 3.01E−20 | 8.27E−18 |
| ENSG00000182324 | KCNJ14 | 0.841577 | 5.55E−05 | 0.000837086 |
| ENSG00000122133 | PAEP | 0.840479 | 0.000151 | 0.001953909 |
| ENSG00000226380 | AC016831.1 | 0.837537 | 3.48E−18 | 6.86E−16 |
| ENSG00000157168 | NRG1 | 0.83576 | 5.15E−06 | 0.000107645 |
| ENSG00000146648 | EGFR | 0.830474 | 0.001072 | 0.010217478 |
| ENSG00000144824 | PHLDB2 | 0.827658 | 2.15E−23 | 8.87E−21 |
| ENSG00000112183 | RBM24 | 0.823749 | 0.000485 | 0.005293307 |
| ENSG00000086730 | LAT2 | 0.821535 | 2.48E−05 | 0.000419187 |
| ENSG00000136244 | IL6 | 0.82091 | 1.54E−05 | 0.000276391 |
| ENSG00000105929 | ATP6V0A4 | 0.820715 | 2.47E−09 | 1.09E−07 |
| ENSG00000138772 | ANXA3 | 0.816579 | 1.93E−14 | 2.20E−12 |
| ENSG00000099834 | CDHR5 | 0.814367 | 0.002871 | 0.022480313 |
| ENSG00000122912 | SLC25A16 | 0.814304 | 4.27E−11 | 2.85E−09 |
| ENSG00000205426 | KRT81 | 0.813905 | 4.03E−13 | 4.02E−11 |
| ENSG00000197632 | SERPINB2 | 0.812676 | 3.88E−10 | 2.13E−08 |
| ENSG00000226312 | CFLAR-AS1 | 0.812436 | 0.003221 | 0.024596348 |
| ENSG00000188372 | ZP3 | 0.809896 | 1.38E−05 | 0.00025212 |
| ENSG00000162413 | KLHL21 | 0.805937 | 1.02E−20 | 2.97E−18 |
| ENSG00000072041 | SLC6A15 | 0.805333 | 6.32E−16 | 9.15E−14 |
| ENSG00000145194 | ECE2 | 0.801873 | 5.64E−15 | 6.90E−13 |
| ENSG00000271643 | AC112220.4 | 0.801342 | 0.000563 | 0.005968173 |
| ENSG00000087074 | PPP1R15A | 0.800673 | 6.79E−21 | 2.08E−18 |
| ENSG00000140941 | MAP1LC3B | 0.79685 | 5.07E−11 | 3.34E−09 |
| ENSG00000162458 | FBLIM1 | 0.796693 | 3.94E−06 | 8.51E−05 |
| ENSG00000085563 | ABCB1 | 0.795488 | 0.00161 | 0.014216484 |
| ENSG00000164604 | GPR85 | 0.792263 | 6.60E−05 | 0.000979121 |
| ENSG00000135549 | PKIB | 0.791705 | 0.000124 | 0.001658726 |
| ENSG00000179913 | B3GNT3 | 0.790419 | 1.93E−06 | 4.53E−05 |
| ENSG00000125637 | PSD4 | 0.789867 | 3.23E−07 | 9.07E−06 |
| ENSG00000187601 | MAGEH1 | 0.78583 | 3.46E−13 | 3.47E−11 |
| ENSG00000166073 | GPR176 | 0.784092 | 1.34E−15 | 1.83E−13 |
| ENSG00000146054 | TRIM7 | 0.778693 | 0.001746 | 0.015142687 |
| ENSG00000132846 | ZBED3 | 0.777962 | 0.005139 | 0.034884523 |
| ENSG00000070669 | ASNS | 0.776597 | 1.97E−17 | 3.46E−15 |
| ENSG00000075618 | FSCN1 | 0.776571 | 4.27E−12 | 3.61E−10 |
| ENSG00000095383 | TBC1D2 | 0.770926 | 1.92E−10 | 1.13E−08 |
| ENSG00000167601 | AXL | 0.770137 | 7.97E−16 | 1.14E−13 |
| ENSG00000167286 | CD3D | 0.767018 | 0.001958 | 0.016575049 |
| ENSG00000153234 | NR4A2 | 0.766436 | 8.21E−05 | 0.001171665 |
| ENSG00000070182 | SPTB | 0.765622 | 5.01E−05 | 0.000765277 |
| ENSG00000180071 | ANKRD18A | 0.764918 | 0.000121 | 0.001617678 |
| ENSG00000110031 | LPXN | 0.763282 | 4.82E−14 | 5.28E−12 |
| ENSG00000115008 | IL1A | 0.763225 | 6.09E−05 | 0.000908765 |
| ENSG00000127528 | KLF2 | 0.760239 | 3.47E−06 | 7.60E−05 |
| ENSG00000187193 | MT1X | 0.758114 | 1.01E−10 | 6.31E−09 |
| ENSG00000268001 | CARD8-AS1 | 0.757313 | 4.81E−06 | 0.000101283 |
| ENSG00000225339 | AL354740.1 | 0.756706 | 0.007588 | 0.047658018 |
| ENSG00000169085 | C8orf46 | 0.755657 | 0.000119 | 0.001597641 |
| ENSG00000258754 | LINC01579 | 0.755548 | 3.68E−05 | 0.000587205 |
| ENSG00000117472 | TSPAN1 | 0.751752 | 0.002321 | 0.019008408 |
| ENSG00000140678 | ITGAX | 0.75009 | 1.18E−05 | 0.000219793 |
| ENSG00000119986 | AVPI1 | 0.746885 | 9.77E−08 | 3.05E−06 |
| ENSG00000260896 | LINC02170 | 0.745531 | 3.19E−12 | 2.77E−10 |
| ENSG00000153237 | CCDC148 | 0.744986 | 0.000583 | 0.006145004 |
| ENSG00000110721 | CHKA | 0.740153 | 3.48E−10 | 1.95E−08 |
| ENSG00000268713 | AC005261.3 | 0.73909 | 0.007945 | 0.049452825 |
| ENSG00000197872 | FAM49A | 0.737795 | 3.69E−08 | 1.25E−06 |
| ENSG00000082497 | SERTAD4 | 0.73707 | 1.90E−06 | 4.48E−05 |
| ENSG00000231298 | LINC00704 | 0.736628 | 0.006002 | 0.039667254 |
| ENSG00000113739 | STC2 | 0.733878 | 6.83E−17 | 1.14E−14 |
| ENSG00000178695 | KCTD12 | 0.729786 | 9.56E−09 | 3.67E−07 |
| ENSG00000134955 | SLC37A2 | 0.729359 | 5.45E−12 | 4.43E−10 |
| ENSG00000101680 | LAMA1 | 0.727109 | 1.36E−10 | 8.26E−09 |
| ENSG00000076513 | ANKRD13A | 0.722988 | 1.56E−16 | 2.39E−14 |
| ENSG00000183496 | MEX3B | 0.72291 | 0.003386 | 0.025509892 |
| ENSG00000179698 | WDR97 | 0.719459 | 0.000141 | 0.001840288 |
| ENSG00000156510 | HKDC1 | 0.716983 | 1.43E−10 | 8.68E−09 |
| ENSG00000133134 | BEX2 | 0.715727 | 4.92E−10 | 2.61E−08 |
| ENSG00000177606 | JUN | 0.715009 | 1.08E−12 | 1.01E−10 |
| ENSG00000261150 | EPPK1 | 0.714793 | 0.005034 | 0.034383881 |
| ENSG00000234465 | PINLYP | 0.713749 | 0.005466 | 0.036611923 |
| ENSG00000163291 | PAQR3 | 0.713377 | 4.99E−18 | 9.45E−16 |
| ENSG00000177181 | RIMKLA | 0.71102 | 0.000677 | 0.006912314 |
| ENSG00000053524 | MCF2L2 | 0.709979 | 0.007729 | 0.048363104 |
| ENSG00000187801 | ZFP69B | 0.709262 | 1.55E−06 | 3.74E−05 |
| ENSG00000117595 | IRF6 | 0.707798 | 1.25E−09 | 5.98E−08 |
| ENSG00000166394 | CYB5R2 | 0.706694 | 0.00051 | 0.005505671 |
| ENSG00000106034 | CPED1 | 0.703495 | 1.37E−07 | 4.11E−06 |
| ENSG00000166246 | C16orf71 | 0.702636 | 0.004719 | 0.032704324 |
| ENSG00000171402 | XAGE3 | 0.699577 | 0.000964 | 0.00933961 |
| ENSG00000186472 | PCLO | 0.697384 | 1.35E−05 | 0.000247045 |
| ENSG00000179598 | PLD6 | 0.696003 | 1.70E−06 | 4.05E−05 |
| ENSG00000133639 | BTG1 | 0.690026 | 7.37E−07 | 1.91E−05 |
| ENSG00000276107 | AC037198.1 | 0.68961 | 0.004307 | 0.030551353 |
| ENSG00000135318 | NT5E | 0.688863 | 1.60E−16 | 2.43E−14 |
| ENSG00000196696 | AC009022.1 | 0.68814 | 8.66E−05 | 0.001224923 |
| ENSG00000259345 | AC013652.1 | 0.685853 | 0.005949 | 0.039410672 |
| ENSG00000185022 | MAFF | 0.684728 | 1.42E−09 | 6.68E−08 |
| ENSG00000162772 | ATF3 | 0.68271 | 7.39E−07 | 1.91E−05 |
| ENSG00000139508 | SLC46A3 | 0.681626 | 0.000957 | 0.009294322 |
| ENSG00000211772 | TRBC2 | 0.681468 | 3.18E−05 | 0.000522234 |
| ENSG00000170989 | S1PR1 | 0.680516 | 7.37E−05 | 0.001068412 |
| ENSG00000151014 | NOCT | 0.679055 | 5.30E−09 | 2.16E−07 |
| ENSG00000154319 | FAM167A | 0.677121 | 4.08E−09 | 1.71E−07 |
| ENSG00000181218 | HIST3H2A | 0.67616 | 1.24E−10 | 7.68E−09 |
| ENSG00000006459 | KDM7A | 0.675097 | 2.64E−10 | 1.52E−08 |
| ENSG00000175573 | C11orf68 | 0.672966 | 4.10E−11 | 2.77E−09 |
| ENSG00000167767 | KRT80 | 0.669671 | 6.69E−08 | 2.15E−06 |
| ENSG00000224959 | AC017002.1 | 0.667922 | 0.002618 | 0.020902531 |
| ENSG00000006606 | CCL26 | 0.666661 | 8.79E−05 | 0.00123778 |
| ENSG00000196368 | NUDT11 | 0.66545 | 9.63E−06 | 0.000183568 |
| ENSG00000144655 | CSRNP1 | 0.663722 | 9.22E−11 | 5.89E−09 |
| ENSG00000117597 | DIEXF | 0.661093 | 2.36E−16 | 3.55E−14 |
| ENSG00000188511 | C22orf34 | 0.661019 | 4.08E−05 | 0.0006401 |
| ENSG00000141458 | NPC1 | 0.658569 | 2.82E−08 | 9.86E−07 |
| ENSG00000229939 | AL589880.1 | 0.657223 | 0.00375 | 0.027498203 |
| ENSG00000125378 | BMP4 | 0.65701 | 0.001815 | 0.015632737 |
| ENSG00000131019 | ULBP3 | 0.655166 | 0.000945 | 0.009205853 |
| ENSG00000104907 | TRMT1 | 0.654994 | 1.69E−13 | 1.75E−11 |
| ENSG00000167646 | DNAAF3 | 0.654731 | 3.34E−05 | 0.000543195 |
| ENSG00000179431 | FJX1 | 0.653506 | 1.06E−09 | 5.18E−08 |
| ENSG00000197608 | ZNF841 | 0.652288 | 2.93E−10 | 1.66E−08 |
| ENSG00000065621 | GSTO2 | 0.650325 | 0.000207 | 0.002572131 |
| ENSG00000171970 | ZNF57 | 0.649859 | 0.001036 | 0.009954619 |
| ENSG00000147676 | MAL2 | 0.649759 | 5.98E−09 | 2.41E−07 |
| ENSG00000181873 | IBA57 | 0.64965 | 6.00E−07 | 1.58E−05 |
| ENSG00000158125 | XDH | 0.645136 | 0.00067 | 0.006848634 |
| ENSG00000196352 | CD55 | 0.645024 | 1.34E−13 | 1.41E−11 |
| ENSG00000256806 | C17orf100 | 0.644038 | 0.001054 | 0.010096067 |
| ENSG00000273038 | AL365203.2 | 0.64226 | 0.00165 | 0.01449452 |
| ENSG00000156535 | CD109 | 0.641792 | 6.15E−09 | 2.47E−07 |
| ENSG00000185338 | SOCS1 | 0.640159 | 0.006265 | 0.041085389 |
| ENSG00000169271 | HSPB3 | 0.638532 | 1.53E−05 | 0.000275608 |
| ENSG00000163818 | LZTFL1 | 0.638395 | 2.00E−10 | 1.17E−08 |
| ENSG00000106366 | SERPINE1 | 0.638272 | 5.10E−10 | 2.68E−08 |
| ENSG00000168811 | IL12A | 0.634991 | 0.001109 | 0.010485351 |
| ENSG00000154589 | LY96 | 0.634482 | 2.38E−06 | 5.48E−05 |
| ENSG00000196155 | PLEKHG4 | 0.632127 | 5.13E−15 | 6.40E−13 |
| ENSG00000133169 | BEX1 | 0.631601 | 1.40E−10 | 8.51E−09 |
| ENSG00000096696 | DSP | 0.63074 | 2.82E−08 | 9.86E−07 |
| ENSG00000279541 | AC005261.5 | 0.630184 | 0.00141 | 0.012716675 |
| ENSG00000146281 | PM20D2 | 0.62892 | 3.12E−08 | 1.07E−06 |
| ENSG00000185697 | MYBL1 | 0.626561 | 2.81E−11 | 1.99E−09 |
| ENSG00000143507 | DUSP10 | 0.626358 | 5.35E−13 | 5.22E−11 |
| ENSG00000113645 | WWC1 | 0.626111 | 9.22E−11 | 5.89E−09 |
| ENSG00000256223 | ZNF10 | 0.625496 | 4.13E−05 | 0.000647488 |
| ENSG00000073711 | PPP2R3A | 0.625288 | 6.53E−10 | 3.37E−08 |
| ENSG00000229056 | AC020571.1 | 0.624348 | 0.000651 | 0.006686647 |
| ENSG00000129474 | AJUBA | 0.624058 | 2.13E−14 | 2.42E−12 |
| ENSG00000150782 | IL18 | 0.621193 | 6.97E−06 | 0.00013879 |
| ENSG00000257605 | AC073611.1 | 0.620706 | 0.001744 | 0.01513957 |
| ENSG00000280213 | UCKL1-AS1 | 0.620139 | 0.001826 | 0.015711546 |
| ENSG00000086696 | HSD17B2 | 0.619526 | 5.13E−08 | 1.70E−06 |
| ENSG00000105327 | BBC3 | 0.619011 | 4.07E−09 | 1.71E−07 |
| ENSG00000145990 | GFOD1 | 0.618714 | 1.39E−09 | 6.55E−08 |
| ENSG00000214814 | FER1L6 | 0.618079 | 0.004614 | 0.032113693 |
| ENSG00000137962 | ARHGAP29 | 0.617815 | 5.09E−10 | 2.68E−08 |
| ENSG00000104419 | NDRG1 | 0.617547 | 2.15E−12 | 1.90E−10 |
| ENSG00000052749 | RRP12 | 0.614025 | 4.01E−09 | 1.69E−07 |
| ENSG00000136997 | MYC | 0.612116 | 0.000404 | 0.004514559 |
| ENSG00000105856 | HBP1 | 0.611567 | 7.93E−10 | 4.06E−08 |
| ENSG00000127914 | AKAP9 | 0.608976 | 2.08E−13 | 2.12E−11 |
| ENSG00000197915 | HRNR | 0.608364 | 4.07E−06 | 8.73E−05 |
| ENSG00000265843 | LINC01029 | 0.608098 | 3.32E−11 | 2.32E−09 |
| ENSG00000105499 | PLA2G4C | 0.608019 | 0.000475 | 0.00520416 |
| ENSG00000164535 | DAGLB | 0.608011 | 9.97E−11 | 6.28E−09 |
| ENSG00000260941 | LINC00622 | 0.607856 | 0.003155 | 0.024201071 |
| ENSG00000222724 | RNU2-63P | 0.605526 | 0.003549 | 0.02639039 |
| ENSG00000269906 | AL606834.1 | 0.604301 | 0.006971 | 0.044531178 |
| ENSG00000158109 | TPRG1L | 0.603191 | 1.79E−08 | 6.50E−07 |
| ENSG00000215218 | UBE2QL1 | 0.600666 | 0.002801 | 0.022014298 |
| ENSG00000158555 | GDPD5 | 0.600509 | 5.43E−05 | 0.000821026 |
| ENSG00000117983 | MUC5B | 0.598975 | 8.44E−05 | 0.001196072 |
| ENSG00000181773 | GPR3 | 0.598935 | 0.00422 | 0.030125647 |
| ENSG00000176177 | ENTHD1 | 0.598309 | 0.001385 | 0.012544869 |
| ENSG00000148908 | RGS10 | 0.597095 | 5.98E−06 | 0.000122231 |
| ENSG00000126947 | ARMCX1 | 0.596387 | 5.90E−08 | 1.92E−06 |
| ENSG00000279184 | NA | 0.591966 | 2.86E−05 | 0.000477169 |
| ENSG00000177873 | ZNF619 | 0.588426 | 3.61E−06 | 7.88E−05 |
| ENSG00000197385 | ZNF860 | 0.587683 | 6.85E−07 | 1.79E−05 |
| ENSG00000143479 | DYRK3 | 0.587612 | 4.49E−09 | 1.86E−07 |
| ENSG00000184470 | TXNRD2 | 0.586862 | 3.37E−11 | 2.35E−09 |
| ENSG00000150593 | PDCD4 | 0.586007 | 6.03E−06 | 0.000122612 |
| ENSG00000110046 | ATG2A | 0.585833 | 2.26E−06 | 5.24E−05 |
| ENSG00000144136 | SLC20A1 | 0.584985 | 4.08E−11 | 2.77E−09 |
| ENSG00000196754 | S100A2 | 0.580868 | 0.000326 | 0.003781602 |
| ENSG00000197415 | VEPH1 | 0.580335 | 5.49E−07 | 1.46E−05 |

TABLE 2
DAVID Functional Annotation Clustering Analysis of genes that are down-regulated upon TRASH-ASO treatment (Enrichment Score >2)

| Annotation | Enrichment Score: | | | | | | | | | | | |
|---|---|---|---|---|---|---|---|---|---|---|---|---|
| Cluster 1 | 2.96936539103503 | | | | | List | Pop | Pop | Fold | | | |
| Category | Term | Count | % | PValue | Genes | Total | Hits | Total | Enrichment | Bonferroni | Benjamini | FDR |
| GOTERM_{—} | GO: 0005581~collagen | 12 | 2.3 | ###### | ENSG00000196739, | 460 | 92 | 18224 | 5.1674858 | 0.006492 | 0.001085 | 0 |
| CC_DIRECT | trimer | | | | ENSG00000158270, | | | | | | | |
| | | | | | ENSG00000188153, | | | | | | | |
| | | | | | ENSG00000131094, | | | | | | | |
| | | | | | ENSG00000204248, | | | | | | | |
| | | | | | ENSG00000060718, | | | | | | | |
| | | | | | ENSG00000138080, | | | | | | | |
| | | | | | ENSG00000082196, | | | | | | | |
| | | | | | ENSG00000204262, | | | | | | | |
| | | | | | ENSG00000171502, | | | | | | | |
| | | | | | ENSG00000187955, | | | | | | | |
| | | | | | ENSG00000082293 | | | | | | | |
| GOTERM_{—} | GO: 0005788~endoplasmic | 17 | 3.3 | ###### | ENSG00000060718, | 460 | 192 | 18224 | 3.5077899 | 0.009854 | 0.001238 | 0 |
| CC_DIRECT | reticulum lumen | | | | ENSG00000198756, | | | | | | | |
| | | | | | ENSG00000084636, | | | | | | | |
| | | | | | ENSG00000204262, | | | | | | | |
| | | | | | ENSG00000171502, | | | | | | | |
| | | | | | ENSG00000115596, | | | | | | | |
| | | | | | ENSG00000114200, | | | | | | | |
| | | | | | ENSG00000144810, | | | | | | | |
| | | | | | ENSG00000196739, | | | | | | | |
| | | | | | ENSG00000188153, | | | | | | | |
| | | | | | ENSG00000120725, | | | | | | | |
| | | | | | ENSG00000080573, | | | | | | | |
| | | | | | ENSG00000204248, | | | | | | | |
| | | | | | ENSG00000100299, | | | | | | | |
| | | | | | ENSG00000092758, | | | | | | | |
| | | | | | ENSG00000187955, | | | | | | | |
| | | | | | ENSG00000082293 | | | | | | | |
| GOTERM_{—} | GO: 0005201~extracellular | 10 | 1.9 | ###### | ENSG00000196739, | 425 | 67 | 16881 | 5.9283582 | 0.026912 | 0.01364 | 0 |
| MF_DIRECT | matrix structural constituent | | | | ENSG00000188153, | | | | | | | |
| | | | | | ENSG00000204248, | | | | | | | |
| | | | | | ENSG00000080573, | | | | | | | |
| | | | | | ENSG00000060718, | | | | | | | |
| | | | | | ENSG00000133048, | | | | | | | |
| | | | | | ENSG00000204262, | | | | | | | |
| | | | | | ENSG00000171502, | | | | | | | |
| | | | | | ENSG00000187955, | | | | | | | |
| | | | | | ENSG00000082293 | | | | | | | |
| GOTERM_{—} | GO: 0030574~collagen | 9 | 1.7 | ###### | ENSG00000144810, | 436 | 64 | 16792 | 5.4159977 | 0.404694 | 0.086436 | 0.1 |
| BP_DIRECT | catabolic process | | | | ENSG00000156103, | | | | | | | |
| | | | | | ENSG00000188153, | | | | | | | |
| | | | | | ENSG00000204248, | | | | | | | |
| | | | | | ENSG00000080573, | | | | | | | |
| | | | | | ENSG00000102996, | | | | | | | |
| | | | | | ENSG00000060718, | | | | | | | |
| | | | | | ENSG00000204262, | | | | | | | |
| | | | | | ENSG00000082293 | | | | | | | |
| KEGG_{—} | hsa04974: Protein | 10 | 1.9 | ###### | ENSG00000196739, | 200 | 88 | 6879 | 3.9085227 | 0.18799 | 0.021081 | 0 |
| PATHWAY | digestion and absorption | | | | ENSG00000188153, | | | | | | | |
| | | | | | ENSG00000204248, | | | | | | | |
| | | | | | ENSG00000080573, | | | | | | | |
| | | | | | ENSG00000060718, | | | | | | | |
| | | | | | ENSG00000092758, | | | | | | | |
| | | | | | ENSG00000175538, | | | | | | | |
| | | | | | ENSG00000204262, | | | | | | | |
| | | | | | ENSG00000171502, | | | | | | | |
| | | | | | ENSG00000187955 | | | | | | | |
| KEGG_{—} | hsa04151: PI3K-Akt | 21 | 4.1 | 0.002 | ENSG00000198121, | 200 | 345 | 6879 | 2.0936087 | 0.390842 | 0.032538 | 0 |
| PATHWAY | signaling pathway | | | | ENSG00000156475, | | | | | | | |
| | | | | | ENSG00000112964, | | | | | | | |
| | | | | | ENSG00000113578, | | | | | | | |
| | | | | | ENSG00000113721, | | | | | | | |
| | | | | | ENSG00000060718, | | | | | | | |
| | | | | | ENSG00000176533, | | | | | | | |
| | | | | | ENSG00000156427, | | | | | | | |
| | | | | | ENSG00000204262, | | | | | | | |
| | | | | | ENSG00000171502, | | | | | | | |
| | | | | | ENSG00000060566, | | | | | | | |
| | | | | | ENSG00000144668, | | | | | | | |
| | | | | | ENSG00000196739, | | | | | | | |
| | | | | | ENSG00000184371, | | | | | | | |
| | | | | | ENSG00000188153, | | | | | | | |
| | | | | | ENSG00000140285, | | | | | | | |
| | | | | | ENSG00000080573, | | | | | | | |
| | | | | | ENSG00000204248, | | | | | | | |
| | | | | | ENSG00000186469, | | | | | | | |
| | | | | | ENSG00000049130, | | | | | | | |
| | | | | | ENSG00000120156 | | | | | | | |
| GOTERM_{—} | GO: 0030199~collagen | 6 | 1.2 | 0.003 | ENSG00000204248, | 436 | 39 | 16792 | 5.9251941 | 0.999074 | 0.442383 | 0.4 |
| BP_DIRECT | fibril organization | | | | ENSG00000080573, | | | | | | | |
| | | | | | ENSG00000134013, | | | | | | | |
| | | | | | ENSG00000060718, | | | | | | | |
| | | | | | ENSG00000204262, | | | | | | | |
| | | | | | ENSG00000187955 | | | | | | | |
| KEGG_{—} | hsa04512: ECM- | 9 | 1.7 | 0.004 | ENSG00000144668, | 200 | 87 | 6879 | 3.5581034 | 0.542826 | 0.045052 | 0 |
| PATHWAY | receptor interaction | | | | ENSG00000196739, | | | | | | | |
| | | | | | ENSG00000188153, | | | | | | | |
| | | | | | ENSG00000204248, | | | | | | | |
| | | | | | ENSG00000080573, | | | | | | | |
| | | | | | ENSG00000060718, | | | | | | | |
| | | | | | ENSG00000196776, | | | | | | | |
| | | | | | ENSG00000204262, | | | | | | | |
| | | | | | ENSG00000171502 | | | | | | | |
| KEGG_{—} | hsa04611: Platelet | 10 | 1.9 | 0.013 | ENSG00000196739, | 200 | 130 | 6879 | 2.6457692 | 0.94266 | 0.12911 | 0.1 |
| PATHWAY | activation | | | | ENSG00000204248, | | | | | | | |
| | | | | | ENSG00000080573, | | | | | | | |
| | | | | | ENSG00000185532, | | | | | | | |
| | | | | | ENSG00000060718, | | | | | | | |
| | | | | | ENSG00000171564, | | | | | | | |
| | | | | | ENSG00000010810, | | | | | | | |
| | | | | | ENSG00000171557, | | | | | | | |
| | | | | | ENSG00000204262, | | | | | | | |
| | | | | | ENSG00000171502 | | | | | | | |
| KEGG_{—} | hsa05146: Amoebiasis | 8 | 1.6 | 0.033 | ENSG00000119699, | 200 | 106 | 6879 | 2.5958491 | 0.99948 | 0.265535 | 0.2 |
| PATHWAY | | | | | ENSG00000196739, | | | | | | | |
| | | | | | ENSG00000188153, | | | | | | | |
| | | | | | ENSG00000204248, | | | | | | | |
| | | | | | ENSG00000080573, | | | | | | | |
| | | | | | ENSG00000060718, | | | | | | | |
| | | | | | ENSG00000204262, | | | | | | | |
| | | | | | ENSG00000171502 | | | | | | | |
| KEGG_{—} | hsa04510: Focal adhesion | 12 | 2.3 | 0.036 | ENSG00000109339, | 200 | 206 | 6879 | 2.0035922 | 0.999724 | 0.268149 | 0.2 |
| PATHWAY | | | | | ENSG00000144668, | | | | | | | |
| | | | | | ENSG00000196739, | | | | | | | |
| | | | | | ENSG00000188153, | | | | | | | |
| | | | | | ENSG00000204248, | | | | | | | |
| | | | | | ENSG00000080573, | | | | | | | |
| | | | | | ENSG00000113721, | | | | | | | |
| | | | | | ENSG00000060718, | | | | | | | |
| | | | | | ENSG00000010810, | | | | | | | |
| | | | | | ENSG00000150760, | | | | | | | |
| | | | | | ENSG00000204262, | | | | | | | |
| | | | | | ENSG00000171502 | | | | | | | |

| Annotation | Enrichment Score: | | | | | | | | | | | |
|---|---|---|---|---|---|---|---|---|---|---|---|---|
| Cluster 2 | 2.275295923156065 | | | | | List | Pop | Pop | Fold | | | |
| Category | Term | Count | % | PValue | Genes | Total | Hits | Total | Enrichment | Bonferroni | Benjamini | FDR |
| KEGG_{—} | hsa05150: Staphylococcus | 11 | 2.1 | ###### | ENSG00000204257, | 200 | 54 | 6879 | 7.0063889 | 5.73E−04 | 3.21E−04 | #### |
| PATHWAY | aureus infection | | | | ENSG00000205403, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000106804, | | | | | | | |
| | | | | | ENSG00000231389, | | | | | | | |
| | | | | | ENSG00000204287, | | | | | | | |
| | | | | | ENSG00000171557, | | | | | | | |
| | | | | | ENSG00000196735, | | | | | | | |
| | | | | | ENSG00000125730 | | | | | | | |
| GOTERM_{—} | GO: 0002504~antigen | 7 | 1.4 | ###### | ENSG00000204257, | 436 | 17 | 16792 | 15.858608 | 0.006205 | 0.002862 | 0 |
| BP_DIRECT | processing and presentation | | | | ENSG00000223865, | | | | | | | |
| | of peptide or polysaccharide | | | | ENSG00000242574, | | | | | | | |
| | antigen via MHC class II | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa04514: Cell adhesion | 17 | 3.3 | ###### | ENSG00000149294, | 200 | 142 | 6879 | 4.1177113 | 6.41E−04 | 3.21E−04 | #### |
| PATHWAY | molecules (CAMs) | | | | ENSG00000242574, | | | | | | | |
| | | | | | ENSG00000204252, | | | | | | | |
| | | | | | ENSG00000204287, | | | | | | | |
| | | | | | ENSG00000021645, | | | | | | | |
| | | | | | ENSG00000158887, | | | | | | | |
| | | | | | ENSG00000196735, | | | | | | | |
| | | | | | ENSG00000174469, | | | | | | | |
| | | | | | ENSG00000144668, | | | | | | | |
| | | | | | ENSG00000204257, | | | | | | | |
| | | | | | ENSG00000223865, | | | | | | | |
| | | | | | ENSG00000169760, | | | | | | | |
| | | | | | ENSG00000162512, | | | | | | | |
| | | | | | ENSG00000231389, | | | | | | | |
| | | | | | ENSG00000018236, | | | | | | | |
| | | | | | ENSG00000091129, | | | | | | | |
| | | | | | ENSG00000149564 | | | | | | | |
| GOTERM_{—} | GO: 0042613~MHC class II | 7 | 1.4 | ###### | ENSG00000204257, | 460 | 22 | 18224 | 12.605534 | 0.004321 | 8.66E−04 | #### |
| CC_DIRECT | protein complex | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0032395~MHC class II | 6 | 1.2 | ###### | ENSG00000204257, | 425 | 15 | 16881 | 15.888 | 0.014984 | 0.01364 | 0 |
| MF_DIRECT | receptor activity | | | | ENSG00000223865, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05310: Asthma | 7 | 1.4 | ###### | ENSG00000204257, | 200 | 30 | 6879 | 8.0255 | 0.039307 | 0.009253 | 0 |
| PATHWAY | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05323: Rheumatoid | 11 | 2.1 | ###### | ENSG00000119699, | 200 | 88 | 6879 | 4.299375 | 0.044947 | 0.009253 | 0 |
| PATHWAY | arthritis | | | | ENSG00000184371, | | | | | | | |
| | | | | | ENSG00000204257, | | | | | | | |
| | | | | | ENSG00000120659, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000120156, | | | | | | | |
| | | | | | ENSG00000196735 | | | | | | | |
| KEGG_{—} | hsa05416: Viral myocarditis | 9 | 1.7 | ###### | ENSG00000170624, | 200 | 57 | 6879 | 5.4307895 | 0.045217 | 0.009253 | 0 |
| PATHWAY | | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000010810, | | | | | | | |
| | | | | | ENSG00000196735 | | | | | | | |
| KEGG_{—} | hsa05332: Graft-versus- | 7 | 1.4 | ###### | ENSG00000204257, | 200 | 33 | 6879 | 7.2959091 | 0.067219 | 0.011596 | 0 |
| PATHWAY | host disease | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05330: Allograft rejection | 7 | 1.4 | ###### | ENSG00000204257, | 200 | 37 | 6879 | 6.5071622 | 0.124249 | 0.018948 | 0 |
| PATHWAY | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05140: Leishmaniasis | 9 | 1.7 | ###### | ENSG00000119699, | 200 | 71 | 6879 | 4.3599296 | 0.190156 | 0.021081 | 0 |
| PATHWAY | | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735, | | | | | | | |
| | | | | | ENSG00000125730 | | | | | | | |
| KEGG_{—} | hsa04940: Type I diabetes | 7 | 1.4 | 0.001 | ENSG00000204257, | 200 | 42 | 6879 | 5.7325 | 0.233242 | 0.022948 | 0 |
| PATHWAY | mellitus | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05145: Toxoplasmosis | 11 | 2.1 | 0.001 | ENSG00000109339, | 200 | 110 | 6879 | 3.4395 | 0.24084 | 0.022948 | 0 |
| PATHWAY | | | | | ENSG00000119699, | | | | | | | |
| | | | | | ENSG00000204257, | | | | | | | |
| | | | | | ENSG00000126803, | | | | | | | |
| | | | | | ENSG00000179583, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0030666~endocytic | 8 | 1.6 | 0.001 | ENSG00000158270, | 460 | 66 | 18224 | 4.802108 | 0.355871 | 0.03996 | 0 |
| CC_DIRECT | vesicle membrane | | | | ENSG00000132535, | | | | | | | |
| | | | | | ENSG00000163072, | | | | | | | |
| | | | | | ENSG00000223865, | | | | | | | |
| | | | | | ENSG00000231389, | | | | | | | |
| | | | | | ENSG00000204287, | | | | | | | |
| | | | | | ENSG00000115596, | | | | | | | |
| | | | | | ENSG00000196735 | | | | | | | |
| KEGG_{—} | hsa04612: Antigen | 9 | 1.7 | 0.001 | ENSG00000204257, | 200 | 76 | 6879 | 4.0730921 | 0.281498 | 0.025411 | 0 |
| PATHWAY | processing and presentation | | | | ENSG00000126803, | | | | | | | |
| | | | | | ENSG00000179583, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa04672: Intestinal immune | 7 | 1.4 | 0.002 | ENSG00000204257, | 200 | 47 | 6879 | 5.1226596 | 0.382761 | 0.032538 | 0 |
| PATHWAY | network for IgA production | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05321: Inflammatory | 8 | 1.6 | 0.002 | ENSG00000119699, | 200 | 64 | 6879 | 4.299375 | 0.406205 | 0.032538 | 0 |
| PATHWAY | bowel disease (IBD) | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0030658~transport | 6 | 1.2 | 0.003 | ENSG00000223865, | 460 | 38 | 18224 | 6.2553776 | 0.564495 | 0.063862 | 0.1 |
| CC_DIRECT | vesicle membrane | | | | ENSG00000164756, | | | | | | | |
| | | | | | ENSG00000231389, | | | | | | | |
| | | | | | ENSG00000204287, | | | | | | | |
| | | | | | ENSG00000196735, | | | | | | | |
| | | | | | ENSG00000167964 | | | | | | | |
| KEGG_{—} | hsa05320: Autoimmune | 7 | 1.4 | 0.004 | ENSG00000204257, | 200 | 52 | 6879 | 4.6300962 | 0.556219 | 0.045052 | 0 |
| PATHWAY | thyroid disease | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0002503~peptide | 3 | 0.6 | 0.006 | ENSG00000204257, | 436 | 5 | 16792 | 23.108257 | 0.999999 | 0.442383 | 0.4 |
| BP_DIRECT | antigen assembly with | | | | ENSG00000242574, | | | | | | | |
| | MHC class II protein | | | | ENSG00000204287 | | | | | | | |
| | complex | | | | | | | | | | | |
| GOTERM_{—} | GO: 0023026~MHC class II | 4 | 0.8 | 0.007 | ENSG00000204257, | 425 | 16 | 16881 | 9.93 | 0.987649 | 0.364905 | 0.4 |
| MF_DIRECT | protein complex binding | | | | ENSG00000242574, | | | | | | | |
| | | | | | ENSG00000204252, | | | | | | | |
| | | | | | ENSG00000204287 | | | | | | | |
| GOTERM_{—} | GO: 0019886~antigen | 8 | 1.6 | 0.01 | ENSG00000077380, | 436 | 92 | 16792 | 3.3490227 | 1 | 0.536219 | 0.5 |
| BP_DIRECT | processing and presentation | | | | ENSG00000204257, | | | | | | | |
| | of exogenous peptide | | | | ENSG00000223865, | | | | | | | |
| | antigen via MHC class II | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa04145: Phagosome | 11 | 2.1 | 0.011 | ENSG00000137285, | 200 | 150 | 6879 | 2.5223 | 0.922115 | 0.125718 | 0.1 |
| PATHWAY | | | | | ENSG00000077380, | | | | | | | |
| | | | | | ENSG00000158270, | | | | | | | |
| | | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735, | | | | | | | |
| | | | | | ENSG00000125730 | | | | | | | |
| KEGG_{—} | hsa05164: Influenza A | 12 | 2.3 | 0.012 | ENSG00000109339, | 200 | 174 | 6879 | 2.372069 | 0.929765 | 0.125718 | 0.1 |
| PATHWAY | | | | | ENSG00000204257, | | | | | | | |
| | | | | | ENSG00000126803, | | | | | | | |
| | | | | | ENSG00000179583, | | | | | | | |
| | | | | | ENSG00000121858, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000184557, | | | | | | | |
| | | | | | ENSG00000196735 | | | | | | | |
| GOTERM_{—} | GO: 0006955~immune | 19 | 3.7 | 0.026 | ENSG00000196083, | 436 | 421 | 16792 | 1.7381508 | 1 | 0.750201 | 0.7 |
| BP_DIRECT | response | | | | ENSG00000179583, | | | | | | | |
| | | | | | ENSG00000242574, | | | | | | | |
| | | | | | ENSG00000204252, | | | | | | | |
| | | | | | ENSG00000069702, | | | | | | | |
| | | | | | ENSG00000204287, | | | | | | | |
| | | | | | ENSG00000196735, | | | | | | | |
| | | | | | ENSG00000006210, | | | | | | | |
| | | | | | ENSG00000145824, | | | | | | | |
| | | | | | ENSG00000164761, | | | | | | | |
| | | | | | ENSG00000204257, | | | | | | | |
| | | | | | ENSG00000120659, | | | | | | | |
| | | | | | ENSG00000121858, | | | | | | | |
| | | | | | ENSG00000009694, | | | | | | | |
| | | | | | ENSG00000223865, | | | | | | | |
| | | | | | ENSG00000231389, | | | | | | | |
| | | | | | ENSG00000163823, | | | | | | | |
| | | | | | ENSG00000164764, | | | | | | | |
| | | | | | ENSG00000125730 | | | | | | | |
| KEGG_{—} | hsa05152: Tuberculosis | 11 | 2.1 | 0.032 | ENSG00000109339, | 200 | 177 | 6879 | 2.1375424 | 0.99925 | 0.262231 | 0.2 |
| PATHWAY | | | | | ENSG00000119699, | | | | | | | |
| | | | | | ENSG00000204257, | | | | | | | |
| | | | | | ENSG00000179583, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735, | | | | | | | |
| | | | | | ENSG00000125730 | | | | | | | |
| GOTERM_{—} | GO: 0042605~peptide | 4 | 0.8 | 0.032 | ENSG00000223865, | 425 | 28 | 16881 | 5.6742857 | 1 | 0.933291 | 0.9 |
| MF_DIRECT | antigen binding | | | | ENSG00000231389, | | | | | | | |
| | | | | | ENSG00000204287, | | | | | | | |
| | | | | | ENSG00000196735 | | | | | | | |
| GOTERM_{—} | GO: 0071556~integral | 4 | 0.8 | 0.036 | ENSG00000223865, | 460 | 29 | 18224 | 5.4644678 | 0.999994 | 0.473025 | 0.5 |
| CC_DIRECT | component of lumenal side | | | | ENSG00000231389, | | | | | | | |
| | of endoplasmic reticulum | | | | ENSG00000204287, | | | | | | | |
| | membrane | | | | ENSG00000196735 | | | | | | | |
| GOTERM_{—} | GO: 0060333~interferon- | 6 | 1.2 | 0.037 | ENSG00000149294, | 436 | 71 | 16792 | 3.2546841 | 1 | 0.871762 | 0.9 |
| BP_DIRECT | gamma-mediated signaling | | | | ENSG00000179583, | | | | | | | |
| | pathway | | | | ENSG00000223865, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05168: Herpes simplex | 11 | 2.1 | 0.039 | ENSG00000109339, | 200 | 183 | 6879 | 2.067459 | 0.999851 | 0.277784 | 0.3 |
| PATHWAY | infection | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000106804, | | | | | | | |
| | | | | | ENSG00000231389, | | | | | | | |
| | | | | | ENSG00000204287, | | | | | | | |
| | | | | | ENSG00000184557, | | | | | | | |
| | | | | | ENSG00000196735, | | | | | | | |
| | | | | | ENSG00000125730 | | | | | | | |
| KEGG_{—} | hsa05322: Systemic lupus | 9 | 1.7 | 0.04 | ENSG00000204257, | 200 | 134 | 6879 | 2.3101119 | 0.999885 | 0.277784 | 0.3 |
| PATHWAY | erythematosus | | | | ENSG00000223865, | | | | | | | |
| | | | | | ENSG00000242574, | | | | | | | |
| | | | | | ENSG00000204252, | | | | | | | |
| | | | | | ENSG00000106804, | | | | | | | |
| | | | | | ENSG00000231389, | | | | | | | |
| | | | | | ENSG00000204287, | | | | | | | |
| | | | | | ENSG00000196735, | | | | | | | |
| | | | | | ENSG00000125730 | | | | | | | |
| GOTERM_{—} | GO: 0012507~ER to Golgi | 5 | 1 | 0.042 | ENSG00000223865, | 460 | 52 | 18224 | 3.8093645 | 0.999999 | 0.509224 | 0.5 |
| CC_DIRECT | transport vesicle membrane | | | | ENSG00000072310, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0019882~antigen | 5 | 1 | 0.054 | ENSG00000223865, | 436 | 55 | 16792 | 3.50125 | 1 | 0.993431 | 1 |
| BP_DIRECT | processing and presentation | | | | ENSG00000242574, | | | | | | | |
| | | | | | ENSG00000231389, | | | | | | | |
| | | | | | ENSG00000204287, | | | | | | | |
| | | | | | ENSG00000196735 | | | | | | | |
| KEGG_{—} | hsa05166: HTLV-I infection | 13 | 2.5 | 0.064 | ENSG00000135925, | 200 | 254 | 6879 | 1.760374 | 1 | 0.395228 | 0.4 |
| PATHWAY | | | | | ENSG00000113721, | | | | | | | |
| | | | | | ENSG00000242574, | | | | | | | |
| | | | | | ENSG00000204252, | | | | | | | |
| | | | | | ENSG00000204287, | | | | | | | |
| | | | | | ENSG00000196735, | | | | | | | |
| | | | | | ENSG00000115596, | | | | | | | |
| | | | | | ENSG00000119699, | | | | | | | |
| | | | | | ENSG00000204257, | | | | | | | |
| | | | | | ENSG00000198909, | | | | | | | |
| | | | | | ENSG00000223865, | | | | | | | |
| | | | | | ENSG00000163513, | | | | | | | |
| | | | | | ENSG00000231389 | | | | | | | |
| GOTERM_{—} | GO: 0030669~clathrin- | 4 | 0.8 | 0.084 | ENSG00000223865, | 460 | 41 | 18224 | 3.8651113 | 1 | 0.683107 | 0.7 |
| CC_DIRECT | coated endocytic vesicle | | | | ENSG00000231389, | | | | | | | |
| | membrane | | | | ENSG00000204287, | | | | | | | |
| | | | | | ENSG00000196735 | | | | | | | |
| GOTERM_{—} | GO: 0031295~T cell | 5 | 1 | 0.144 | ENSG00000223865, | 436 | 78 | 16792 | 2.4688309 | 1 | 1 | 1 |
| BP_DIRECT | costimulation | | | | ENSG00000231389, | | | | | | | |
| | | | | | ENSG00000204287, | | | | | | | |
| | | | | | ENSG00000010810, | | | | | | | |
| | | | | | ENSG00000196735 | | | | | | | |
| GOTERM_{—} | GO: 0042102~positive | 4 | 0.8 | 0.203 | ENSG00000223865, | 436 | 60 | 16792 | 2.5675841 | 1 | 1 | 1 |
| BP_DIRECT | regulation of T cell | | | | ENSG00000242574, | | | | | | | |
| | proliferation | | | | ENSG00000231389, | | | | | | | |
| | | | | | ENSG00000163554 | | | | | | | |
| GOTERM_{—} | GO: 0031902~late | 5 | 1 | 0.25 | ENSG00000134108, | 460 | 101 | 18224 | 1.961257 | 1 | 1 | 1 |
| CC_DIRECT | endosome membrane | | | | ENSG00000196814, | | | | | | | |
| | | | | | ENSG00000204257, | | | | | | | |
| | | | | | ENSG00000242574, | | | | | | | |
| | | | | | ENSG00000204287 | | | | | | | |
| GOTERM_{—} | GO: 0032588~trans-Golgi | 4 | 0.8 | 0.348 | ENSG00000223865, | 460 | 83 | 18224 | 1.9092719 | 1 | 1 | 1 |
| CC_DIRECT | network membrane | | | | ENSG00000231389, | | | | | | | |
| | | | | | ENSG00000204287, | | | | | | | |
| | | | | | ENSG00000196735 | | | | | | | |
| KEGG_{—} | hsa05169: Epstein-Barr | 5 | 1 | 0.472 | ENSG00000109339, | 200 | 122 | 6879 | 1.4096311 | 1 | 1 | 0.9 |
| PATHWAY | virus infection | | | | ENSG00000223865, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0050852~T cell | 5 | 1 | 0.536 | ENSG00000223865, | 436 | 148 | 16792 | 1.3011406 | 1 | 1 | 1 |
| BP_DIRECT | receptor signaling pathway | | | | ENSG00000231389, | | | | | | | |
| | | | | | ENSG00000204287, | | | | | | | |
| | | | | | ENSG00000010810, | | | | | | | |
| | | | | | ENSG00000196735 | | | | | | | |
| GOTERM_{—} | GO: 0005765~lysosomal | 8 | 1.6 | 0.536 | ENSG00000134108, | 460 | 274 | 18224 | 1.1567122 | 1 | 1 | 1 |
| CC_DIRECT | membrane | | | | ENSG00000204257, | | | | | | | |
| | | | | | ENSG00000223865, | | | | | | | |
| | | | | | ENSG00000242574, | | | | | | | |
| | | | | | ENSG00000204252, | | | | | | | |
| | | | | | ENSG00000231389, | | | | | | | |
| | | | | | ENSG00000204287, | | | | | | | |
| | | | | | ENSG00000196735 | | | | | | | |
| GOTERM_{—} | GO: 0010008~endosome | 4 | 0.8 | 0.848 | ENSG00000223865, | 460 | 185 | 18224 | 0.8565922 | 1 | 1 | 1 |
| CC_DIRECT | membrane | | | | ENSG00000204252, | | | | | | | |
| | | | | | ENSG00000231389, | | | | | | | |
| | | | | | ENSG00000196735 | | | | | | | |

DAVID Functional Annotation Clustering Analysis of genes that are up-regulated upon TRASH-ASO treatment (Enrichment Score >2)

| Annotation | Enrichment Score: | | | | | | | | | | | |
|---|---|---|---|---|---|---|---|---|---|---|---|---|
| Cluster 1 | 2.1206711907738813 | | | | | List | Pop | Pop | Fold | | | |
| Category | Term | Count | % | PValue | Genes | Total | Hits | Total | Enrichment | Bonferroni | Benjamini | FDR |
| GOTERM_{—} | GO: 0004713~protein | 9 | 2.1 | 0.005 | ENSG00000182866, | 334 | 133 | 16881 | 3.4201297 | 0.906337 | 0.337463 | 0.3 |
| MF_DIRECT | tyrosine kinase activity | | | | ENSG00000143479, | | | | | | | |
| | | | | | ENSG00000146648, | | | | | | | |
| | | | | | ENSG00000198223, | | | | | | | |
| | | | | | ENSG00000100368, | | | | | | | |
| | | | | | ENSG00000157168, | | | | | | | |
| | | | | | ENSG00000138675, | | | | | | | |
| | | | | | ENSG00000143322, | | | | | | | |
| | | | | | ENSG00000167601 | | | | | | | |
| GOTERM_{—} | GO: 0005088~Rasguanyl- | 8 | 1.8 | 0.008 | ENSG00000146648, | 334 | 115 | 16881 | 3.5159594 | 0.976867 | 0.41687 | 0.4 |
| MF_DIRECT | nucleotide exchange | | | | ENSG00000058335, | | | | | | | |
| | factor activity | | | | ENSG00000198223, | | | | | | | |
| | | | | | ENSG00000127914, | | | | | | | |
| | | | | | ENSG00000100368, | | | | | | | |
| | | | | | ENSG00000157168, | | | | | | | |
| | | | | | ENSG00000100385, | | | | | | | |
| | | | | | ENSG00000138675 | | | | | | | |
| GOTERM_{—} | GO: 0018108~peptidyl- | 9 | 2.1 | 0.011 | ENSG00000143479, | 334 | 153 | 16792 | 2.9573794 | 1 | 0.894092 | 0.9 |
| BP_DIRECT | tyrosine phosphorylation | | | | ENSG00000108798, | | | | | | | |
| | | | | | ENSG00000146648, | | | | | | | |
| | | | | | ENSG00000198223, | | | | | | | |
| | | | | | ENSG00000100368, | | | | | | | |
| | | | | | ENSG00000157168, | | | | | | | |
| | | | | | ENSG00000138675, | | | | | | | |
| | | | | | ENSG00000143322, | | | | | | | |
| | | | | | ENSG00000167601 | | | | | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A single or double-stranded nucleic acid of 12-50 nucleotides in length comprising at least 12 nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, wherein introduction of the single or double-stranded nucleic acid into a cell expressing long non-coding RNA (lncRNA) BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 inhibits expression of the lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

2. The single or double-stranded nucleic acid of claim 1 comprising at least 12 contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

3. The single or double-stranded nucleic acid of claim 1, wherein the single or double-stranded nucleic acid is a single-stranded nucleic acid that is an antisense polynucleotide or a ribozyme that targets lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

4. The single-stranded nucleic acid of claim 3 comprising the sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO:41 or SEQ ID NO:47.

5. The single or double-stranded nucleic acid of claim 1, wherein the single or double-stranded nucleic acid is a double-stranded nucleic acid that is a small interfering RNA (siRNA) or a short hairpin RNA (shRNA) that targets lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

6. The double-stranded nucleic acid of claim 5 comprising a sense strand and an antisense strand, wherein the sense strand and the antisense comprise the sequence of SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 42 and SEQ ID NO: 50; SEQ ID NO: 43 and SEQ ID NO: 51; SEQ ID NO: 44 and SEQ ID NO: 52; SEQ ID NO: 45 and SEQ ID NO: 53; or SEQ ID NO: 46 and SEQ ID NO: 54.

7. The single or double-stranded nucleic acid of claim 1, wherein the single or double-stranded nucleic acid is a single-stranded nucleic acid that is a guide RNA (gRNA) that targets a polynucleotide encoding lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201.

8. An antisense polynucleotide comprising SEQ ID NO: 48, wherein introduction of the antisense polynucleotide into a cell expressing HNRNPA2/B1 inhibits expression of HNRNPA2/B1 or antisense polynucleotide comprising SEQ ID NO: 49, wherein introduction of the antisense polynucleotide into a cell expressing SNX10 inhibits expression of SNX10.

9. (canceled)

10. The single or double-stranded nucleic acid of claim 1, comprising at least one modified nucleotide.

11. The single or double-stranded nucleic acid of any one of claim 10, wherein the modified nucleotide comprises a modification selected from the group consisting of a sugar modification, a nucleic acid base modification, and a phosphate backbone modification.

12. The single or double-stranded nucleic acid of claim 11, wherein the 2′-sugar modification is selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, and locked nucleic acid (LNA) modification.

13. The single or double-stranded nucleic acid of claim 11, wherein the phosphate backbone modification is a 5′ phosphorylation.

14. The double-stranded nucleic acid of claim 5, wherein the double-stranded nucleic acid and comprises a 1-6 nucleotide overhang.

15. A vector comprising the single or double-stranded nucleic acid of any of claim 1.

16. The vector of claim 15, wherein the vector is a viral vector.

17. The vector of claim 16, wherein the viral vector is a retroviral, a lentiviral, or an adeno-associated viral (AAV) vector.

18. A pharmaceutical composition comprising the single or double-stranded nucleic acid of claim 1 and a pharmaceutically acceptable carrier.

19. The pharmaceutical composition of claim 18, further comprising a specific inhibitor of one or more kinases selected from the group consisting of MEK, PLK1, TAF, AURKA, HER, PTK2, PKD, PKC, IKBK, MAP3K, PIM, SRC, PAK, AKT, ERK, and RAF.

20. (canceled)

21. (canceled)

22. (canceled)

23. A method of inhibiting cancer cell that is dependent on MAPK pathway hyperactivation, the method comprising contacting the single or double-stranded nucleic acid of claim 1 with the cancer cell such that expression of lncRNA BX470102.3-008, AC004540.4-001, AC004540.4-002, RP11-7011.3-001, RP11-7011.3-003, RP11-7011.3-002, RN7SL1-202, RN7SL1-201, ARF-AS1-201, ARF-AS1-202, ARF-AS1-203 or AL157871.4-201 is inhibited.

24-28. (canceled)

29. A method of inhibiting a cancer cell that is dependent on MAPK pathway hyperactivation cancer cell, the method comprising contacting the cancer cell with a specific inhibitor of one or more kinases selected from the group consisting of MEK, PLK1, TAF, AURKA, HER, PTK2, PKD, PKC, IKBK, MAP3K, PIM, SRC, PAK, AKT, ERK, and RAF in an amount to inhibit the cancer cell growth.

30-37. (canceled)


SEQUENCE LISTING (54 sequences)

SEQ ID NO: 1 - DNA, 531 bp, Homo sapiens
  misc_feature 1..531: note = Gene name: BX470102.3-008
  source 1..531: mol_type = genomic DNA
ccccacccagtggggacagaagacaacttaattccacaaagttggacccccaagggaagtggggaggtgagagggaaagaaggatgtggtctccatctaaagtcaagatctcttccccagaagctagaggtaaacttgcccagctcggactaggcctagagaggctgaatgatgtggcatcacccggaaacagcgtttaccctccttatcctcttcccttctgcctgaaaacactaatccagatgatggacaatgattcaattagtcatcttctctgatgggggctgagatccaggctgggatcccgtggaagtgccgggcaatcctctctgcaagtggctctgtgctccctcatcaccaaggacccatgtcactttggcattgcttctcctcagctacttctcagttactggtcctcatttggagagatggatatccggctggaagcatcccctacccgctgggagagtgggtctacagctcagggtctacatgtggaccagggcctcagaatgtgggtaaatgtgagtc

SEQ ID NO: 2 - DNA, 611 bp, Homo sapiens
  misc_feature 1..611: note = Gene name: AC004540.4-001
  source 1..611: mol_type = genomic DNA
agcccagtcgccgccgccagcggagcggcccgggcgggacgcggcgggagcgcgcgtgtgcgggacgcagcgcgggggatgcgcgcgggccgcggaggcgccgcaaccaacaggcggccgagggtgcagccgcgggagcgccaccgccagtggggtggggggcaaagctataaagaaggcccagaggattcctgcggagctgtatttactttacatccatgtgaactgctgtcatcactactgtgtccaagcccagaggatgaactggaaaagaagagagggggaaaataataaaaagaggaaattggttttcacaacacactcaaagcctgagtaacagaggagaactttaattatctccagtcacaaagagagacaggaaatttggacttttaattagccatttggagtgcagttgggtatttttttagctagataatttaaacgcgaataattcaagtctgactaaatgaaagtcacataatcagaatgcaaataattgaatttctactgcattcattaattcagtgtggaggtgtgtgtgaagactactatgatgagctgtcacagctcaataaaatctcagtcaattaattttttcattatcttag

SEQ ID NO: 3 - DNA, 508 bp, Homo sapiens
  misc_feature 1..508: note = Gene name: AC004540.4-002
  source 1..508: mol_type = genomic DNA
ccgcccctcaaccaacccccaggcggcaaggccctctccacgcgcgctctccagcctggcggggccctggaagccgggacacgcggaggccgggagggtcatcggcgtttaaggcagcctcccacaccaagtgcaccgcccggatcccctctgcacgagggctttctgcttattgctcttttccccagcagccagaatcgtcaccgtagcgcgggaaggggcctcgcgggccgtctgcagcaggtgcggccgggagccgcaggcccgcgcggattcctgcggagctgtatttactttacatccatgtgaactgctgtcatcactactgtgtccaagcccagaggatgaactggaaaagaagagagggggaaaataataaaaagaggaaattggttttcacaacacactcaaagcctgagtaacagaggagaactttaattatctccagtcacaaagagagacaggaaatttggacttttaattagccatttggagtgcagttgggtat

SEQ ID NO: 4 - DNA, 486 bp, Homo sapiens
  misc_feature 1..486: note = Gene name: RP11-7011.3-001
  source 1..486: mol_type = genomic DNA
gggaccacccaataagcaaccgggaaaccacataccatggacagtgctcggggaatttcggctcctcctcatccctggaggttccagggatctggcagagaaagaagcccagtctcacagcaatgactcctgaacatcctttgcaaacactatttaggacagatggactgaagtaggccctggggagagttgggggcatttccggaggccagaccaaaggtgatctccaggagatttggatgttagtgcaggatagctcagagccaagtggcagataagtctttgggagccagaagggctctttcttctctaaggaacaagtgtagacctataggggcaagaacctgtggcaaccctgggagaggtctgcagattcaggagggaacatcccaaaactcagcagaggaggagagggagcttttgttggtcacctggaaccactaccaatccatgttcatgtcaaattaaatgatcactttgaagttt

SEQ ID NO: 5 - DNA, 294 bp, Homo sapiens
  misc_feature 1..294: note = Gene name: RP11-7011.3-003
  source 1..294: mol_type = genomic DNA
caaccgggaaaccacataccatggacagtgctcggggaatttcggctcctcctcatccctggaggttccagggatctggcagagaaagaagcccagtctcacagcaatgactcctgaacatcctttgcaaacactatttaggacagatggactgaagtaggccctggggagagttgggggcatttccggagggaacatcccaaaactcagcagaggaggagagggagcttttgttggtcacctggaaccactaccaatccatgttcatgtcaaattaaatgatcactttgaagt

SEQ ID NO: 6 - DNA, 494 bp, Homo sapiens
  misc_feature 1..494: note = Gene name: RP11-7011.3-002
  source 1..494: mol_type = genomic DNA
cagaccggcgcggggcgctgcggcccaactccttagtaggacgacgtgactcgaggggccggaggacggagggctcctcctcatccctggaggttccagggatctggcagagaaagaagcccagtctcacagcaatgactcctgaacatcctttgcaaacactatttaggacagatggactgaagtaggccctggggagagttgggggcatttccggaggccagaccaaaggtgatctccaggagatttggatgttagtgcaggatagctcagagccaagtggcagataagtctttgggagccagaagggctctttcttctctaaggaacaagtgtagacctataggggcaagaacctgtggcaaccctgggagaggtctgcagattcaggagggaacatcccaaaactcagcagaggaggagagggagcttttgttggtcacctggaaccactaccaatccatgttcatgtcaaattaaatgatcactttgaa

SEQ ID NO: 7 - DNA, 300 bp, Homo sapiens
  misc_feature 1..300: note = Gene name: RN7SL1-202
  source 1..300: mol_type = genomic DNA
cgccgggcgcggtggcgcgtgcctgtagtcccagctactcgggaggctgaggctggaggatcgcttgagtccaggagttctgggctgtagtgcgctatgccgatcgggtgtccgcactaagttcggcatcaatatggtgacctcccgggagcgggggaccaccaggttgcctaaggaggggtgaaccggcccaggtcggaaacggagcaggtcaaaactcccgtgctgatcagtagtgggatcgcgcctgtgaatagccactgcactccagcctgggcaacatagcgagaccccgtctct

SEQ ID NO: 8 - DNA, 299 bp, Homo sapiens
  misc_feature 1..299: note = Gene name: RN7SL1-201
  source 1..299: mol_type = genomic DNA
gccgggcgcggtggcgcgtgcctgtagtcccagctactcgggaggctgaggctggaggatcgcttgagtccaggagttctgggctgtagtgcgctatgccgatcgggtgtccgcactaagttcggcatcaatatggtgacctcccgggagcgggggaccaccaggttgcctaaggaggggtgaaccggcccaggtcggaaacggagcaggtcaaaactcccgtgctgatcagtagtgggatcgcgcctgtgaatagccactgcactccagcctgggcaacatagcgagaccccgtctct

SEQ ID NO: 9 - DNA, 327 bp, Homo sapiens
  misc_feature 1..327: note = Gene name: ARF-AS1-201
  source 1..327: mol_type = genomic DNA
cttgcttccggaaaggcgagctgagcattatgggttagggtctcactttgtcacccaagctgaagtacagtggcatcatctcggcttactcaacctcctgggatcaagtgatcctcccacctcagcccccaaagtagctgggactacaggtcaggcatggtggctcacacctgtaatcccagcatgttgggaggccaagatgggagactcacttgagcccagaagttccagaccagccttggcaatatagtgagatgccatttctattttaaaaaatattttaaaaataaaatatttttctattcacctttcatcaatacaaaccca

SEQ ID NO: 10 - DNA, 437 bp, Homo sapiens
  misc_feature 1..437: note = Gene name: ARF-AS1-202
  source 1..437: mol_type = genomic DNA
gatgggtattccctgatgccatgaacttacacgtttcacacacgggaccagacgcttgctttagttgacgcatgaagaccggtccggtcttttgcggagaaaagtggttaaaagctgacttgtgggccgagaaactgtggcaccctaatgagctagggctagacgcttcgaccaccacgccaagtgattctgaagatctctaattctgtcaaggcgagagcgctccaacacgtgttcatcggctgttgctttttaaagagaaggtcaggcatggtggctcacacctgtaatcccagcatgttgggaggccaagatgggagactcacttgagcccagaagttccagaccagccttggcaatatagtgagatgccatttctattttaaaaaatattttaaaaataaaatatttttctattcacctttcatcaatacaaa

SEQ ID NO: 11 - DNA, 552 bp, Homo sapiens
  misc_feature 1..552: note = Gene name: ARF-AS1-203
  source 1..552: mol_type = genomic DNA
attccctgatgccatgaacttacacgtttcacacacgggaccagacgcttgctttagttgacgcatgaagaccggtccggtcttttgcggagaaaagtggttaaaagctgacttgtgggccgagaaactgtggcaccctaatgagctagggctagacgcttcgaccaccacgccaagtgattctgaagatctctaattctgtcaaggcgagagcgctccaacacgtgttcatcggctgttgctttttaaagagaagggtctcactttgtcacccaagctgaagtacagtggcatcatctcggcttactcaacctcctgggatcaagtgatcctcccacctcagcccccaaagtagctgggactacaggtcaggcatggtggctcacacctgtaatcccagcatgttgggaggccaagatgggagactcacttgagcccagaagttccagaccagccttggcaatatagtgagatgccatttctattttaaaaaatattttaaaaataaaatatttttctattcacctttcatcaatacaaacccagaagaga

SEQ ID NO: 12 - DNA, 385 bp, Homo sapiens
  misc_feature 1..385: note = Gene name: AL157871.4-201
  source 1..385: mol_type = genomic DNA
caggagccaaggaagtttatttactctactgggtgacaggagggcagagtgctccagaggagacccagatacatcaaccaaggacttccctgagatttggctttgctcttccagggtcaggtccttccacagatacttctcgtcatccgtcatctggatgaccaagggcacgttaaatacatcctggagccacctaaagaaacacagggggagaaagctgacgtctcatctcccctgtggaggaacgccatcgtgcatctgaaaacacagctcctacttacaacgtatgttaaaacttccttgcctacaaaatcacaatttgaattgtgatatgaatttgactatggataatgataaggtctactaccatttatcgaccaggtgg

SEQ ID NO: 13 - DNA, 16 bp, synthetic construct
  misc_feature 1..16: note = Description of Sequence: Antisense oligonucleotide sequence targeting BX470102.3-008
  source 1..16: mol_type = other DNA
aaggagggtaaacgct

SEQ ID NO: 14 - DNA, 16 bp, synthetic construct
  misc_feature 1..16: note = Description of Sequence: Antisense oligonucleotide sequence targeting BX470102.3-008
  source 1..16: mol_type = other DNA
atcatctggattagtg

SEQ ID NO: 15 - DNA, 16 bp, synthetic construct
  misc_feature 1..16: note = Description of Sequence: Antisense oligonucleotide sequence targeting AC004540.4-001 and AC004540.4-002
  source 1..16: mol_type = other DNA
gactggagataattaa

SEQ ID NO: 16 - DNA, 16 bp, synthetic construct
  misc_feature 1..16: note = Description of Sequence: Antisense oligonucleotide sequence targeting AC004540.4-001 and AC004540.4-002
  source 1..16: mol_type = other DNA
tgcgcggcggaaagaa

SEQ ID NO: 17 - DNA, 16 bp, synthetic construct
  misc_feature 1..16: note = Description of Sequence: Antisense oligonucleotide sequence targeting RP11-7011.3-001 002 and 003
  source 1..16: mol_type = other DNA
acatggattggtagtg

SEQ ID NO: 18 - DNA, 16 bp, synthetic construct
  misc_feature 1..16: note = Description of Sequence: Antisense oligonucleotide sequence targeting RP11-7011.3-001 002 and 003
  source 1..16: mol_type = other DNA
gatcatttaatttgac

SEQ ID NO: 19 - DNA, 16 bp, synthetic construct
  misc_feature 1..16: note = Description of Sequence: Antisense oligonucleotide sequence targeting ARF-AS1-201 ARF-AS1-202 and ARF-AS1-203
  source 1..16: mol_type = other DNA
attgatgaaaggtgaa

SEQ ID NO: 20 - DNA, 16 bp, synthetic construct
  misc_feature 1..16: note = Description of Sequence: Antisense oligonucleotide sequence targeting ARF-AS1-202 and ARF-AS1-203
  source 1..16: mol_type = other DNA
gcgtcaactaaagcaa

SEQ ID NO: 21 - DNA, 16 bp, synthetic construct
  misc_feature 1..16: note = Description of Sequence: Antisense oligonucleotide sequence targeting AL157871.4-201
  source 1..16: mol_type = other DNA
aagtccttggttgatg

SEQ ID NO: 22 - DNA, 16 bp, synthetic construct
  misc_feature 1..16: note = Description of Sequence: Antisense oligonucleotide sequence targeting AL157871.4-201
  source 1..16: mol_type = other DNA
gtaagtaggagctgtg

SEQ ID NO: 23 - RNA, 21 nt, synthetic construct
  misc_feature 1..21: note = Description of Sequence: siRNA sense strand sequence targeting BX470102.3-008
  source 1..21: mol_type = other RNA
cccagaagctagaggtaaatt

SEQ ID NO: 24 - RNA, 21 nt, synthetic construct
  misc_feature 1..21: note = Description of Sequence: siRNA antisense strand sequence targeting BX470102.3-008
  source 1..21: mol_type = other RNA
tttacctctagcttctgggtt

SEQ ID NO: 25 - RNA, 21 nt, synthetic construct
  misc_feature 1..21: note = Description of Sequence: siRNA sense strand sequence targeting AC004540.4-001 and AC004540.4-002
  source 1..21: mol_type = other RNA
gagtaacagaggagaactttt

SEQ ID NO: 26 - RNA, 21 nt, synthetic construct
  misc_feature 1..21: note = Description of Sequence: siRNA antisense strand sequence targeting AC004540.4-001 and AC004540.4-002
  source 1..21: mol_type = other RNA
aagttctcctctgttactctt

SEQ ID NO: 27 - RNA, 21 nt, synthetic construct
  misc_feature 1..21: note = Description of Sequence: siRNA sense strand sequence targeting both isoforms of RP11-7011.3-001 002 and 003
  source 1..21: mol_type = other RNA
agcaatgactcctgaacattt

SEQ ID NO: 28 - RNA, 21 nt, synthetic construct
  misc_feature 1..21: note = Description of Sequence: siRNA antisense strand sequence targeting both isoforms of RP11-7011.3-001 002 and 003
  source 1..21: mol_type = other RNA
atgttcaggagtcattgcttt

SEQ ID NO: 29 - RNA, 21 nt, synthetic construct
  misc_feature 1..21: note = Description of Sequence: siRNA sense strand sequence targeting RN7SL1-201 and 202
  source 1..21: mol_type = other RNA
gcactaagttcggcatcaatt

SEQ ID NO: 30 - RNA, 19 nt, synthetic construct
  misc_feature 1..19: note = Description of Sequence: siRNA antisense strand sequence targeting RN7SL1
  source 1..19: mol_type = other RNA
ttgatgccgaacttagtgc

SEQ ID NO: 31 - RNA, 21 nt, synthetic construct
  misc_feature 1..21: note = Description of Sequence: siRNA sense strand sequence targeting RN7SL1
  source 1..21: mol_type = other RNA
actaagttcggcatcaatatt

SEQ ID NO: 32 - RNA, 19 nt, synthetic construct
  misc_feature 1..19: note = Description of Sequence: siRNA antisense strand sequence targeting RN7SL1
  source 1..19: mol_type = other RNA
tattgatgccgaacttagt

SEQ ID NO: 33 - RNA, 21 nt, synthetic construct
  misc_feature 1..21: note = Description of Sequence: siRNA sense strand sequence targeting RN7SL1
  source 1..21: mol_type = other RNA
ggaccaccaggttgcctaatt

SEQ ID NO: 34 - RNA, 19 nt, synthetic construct
  misc_feature 1..19: note = Description of Sequence: siRNA antisense strand sequence targeting RN7SL1
  source 1..19: mol_type = other RNA
ttaggcaacctggtggtcc

SEQ ID NO: 35 - RNA, 21 nt, synthetic construct
  misc_feature 1..21: note = Description of Sequence: siRNA sense strand sequence targeting RN7SL1
  source 1..21: mol_type = other RNA
gggaccaccaggttgcctatt

SEQ ID NO: 36 - RNA, 19 nt, synthetic construct
  misc_feature 1..19: note = Description of Sequence: siRNA antisense strand sequence targeting RN7SL1
  source 1..19: mol_type = other RNA
taggcaacctggtggtccc

SEQ ID NO: 37 - RNA, 21 nt, synthetic construct
  misc_feature 1..21: note = Description of Sequence: siRNA sense strand sequence targeting ARF-AS1
  source 1..21: mol_type = other RNA
ggaaaggcgagctgagcattt

SEQ ID NO: 38 - RNA, 21 nt, synthetic construct
  misc_feature 1..21: note = Description of Sequence: siRNA antisense strand sequence targeting ARF-AS1 isoform 1
  source 1..21: mol_type = other RNA
atgctcagctcgcctttcctt

SEQ ID NO: 39 - RNA, 21 nt, synthetic construct
  misc_feature 1..21: note = Description of Sequence: siRNA sense strand sequence targeting AL157871.4-201
  source 1..21: mol_type = other RNA
tggataatgataaggtctatt

SEQ ID NO: 40 - RNA, 21 nt, synthetic construct
  misc_feature 1..21: note = Description of Sequence: siRNA antisense strand sequence targeting AL157871.4-201
  source 1..21: mol_type = other RNA
tagaccttatcattatccatt

SEQ ID NO: 41 - DNA, 16 bp, synthetic construct
  misc_feature 1..16: note = Description of Sequence: Antisense oligonucleotide sequence: targeting AC004540.4-001 and AC004540.4-002
  source 1..16: mol_type = other DNA
ctcatgagctgtcgta

SEQ ID NO: 42 - RNA, 25 nt, synthetic construct
  misc_feature 1..25: note = Description of Sequence: Duplex Sequence: targeting AC004540.4-001 and AC004540.4-002
  source 1..25: mol_type = other RNA
ggtaaaaacatgaagctaatagtta

SEQ ID NO: 43 - RNA, 25 nt, synthetic construct
  misc_feature 1..25: note = Description of Sequence: Duplex Sequence: targeting AC004540.4-001 and AC004540.4-002
  modified_base 25: mod_base = OTHER; note = Thymine
  source 1..25: mol_type = other RNA
gccaacagcatgtcaattcagtgat

SEQ ID NO: 44 - RNA, 25 nt, synthetic construct
  misc_feature 1..25: note = Description of Sequence: Duplex Sequence: targeting AC004540.4-001 and AC004540.4-002
  modified_base 25: mod_base = OTHER; note = Thymine
  source 1..25: mol_type = other RNA
atttcatgtctgaagcaattctact

SEQ ID NO: 45 - RNA, 21 nt, synthetic construct
  misc_feature 1..21: note = Description of Sequence: Duplex Sequence: targeting AC004540.4-001 and AC004540.4-002
  source 1..21: mol_type = other RNA
acaaagagagacaggaaattt

SEQ ID NO: 46 - RNA, 21 nt, synthetic construct
  misc_feature 1..21: note = Description of Sequence: Duplex Sequence: targeting AC004540.4-001 and AC004540.4-002
  source 1..21: mol_type = other RNA
tcacaaagagagacaggaatt

SEQ ID NO: 47 - DNA, 16 bp, synthetic construct
  misc_feature 1..16: note = Description of Sequence: Antisense oligonucleotide sequence targeting AC004540.4-001 and AC004540.4-002
  modified_base 16: mod_base = OTHER; note = Cholesterol modification
  source 1..16: mol_type = other DNA
gactggagataattaa

SEQ ID NO: 48 - DNA, 16 bp, synthetic construct
  misc_feature 1..16: note = Description of Sequence: Antisense oligonucleotide sequence: targeting HNRNPA2/B1
  source 1..16: mol_type = other DNA
gaccgtagttagaagg

SEQ ID NO: 49 - DNA, 16 bp, synthetic construct
  misc_feature 1..16: note = Description of Sequence: Antisense oligonucleotide sequence: targeting SNX10
  source 1..16: mol_type = other DNA
agatggctctgtaaga

SEQ ID NO: 50 - RNA, 27 nt, synthetic construct
  misc_feature 1..27: note = Description of Sequence: Duplex Sequence: targeting AC004540.4-001 and AC004540.4-002
  source 1..27: mol_type = other RNA
acccatttttgtacttcgattatcaat

SEQ ID NO: 51 - RNA, 27 nt, synthetic construct
  misc_feature 1..27: note = Description of Sequence: Duplex Sequence: targeting AC004540.4-001 and AC004540.4-002
  source 1..27: mol_type = other RNA
gacggttgtcgtacagttaagtcacta

SEQ ID NO: 52 - RNA, 27 nt, synthetic construct
  misc_feature 1..27: note = Description of Sequence: Duplex Sequence: targeting AC004540.4-001 and AC004540.4-002
  source 1..27: mol_type = other RNA
aataaagtacagacttcgttaagatga

SEQ ID NO: 53 - RNA, 21 nt, synthetic construct
  misc_feature 1..21: note = Description of Sequence: Duplex Sequence: targeting AC004540.4-001 and AC004540.4-002
  source 1..21: mol_type = other RNA
atttcctgtctctctttgttt

SEQ ID NO: 54 - RNA, 21 nt, synthetic construct
  misc_feature 1..21: note = Description of Sequence: Duplex Sequence: targeting AC004540.4-001 and AC004540.4-002
  source 1..21: mol_type = other RNA
ttcctgtctctctttgtgatt